(19) United States
(12) Patent Application Publication
(10) Pub. No.: US 2022/0193447 A1
(43) Pub. Date: Jun. 23, 2022

(54) METHODS FOR MODULATION OF AND SENSITIZATION TO SERINE AND GLYCINE LIMITATION

(71) Applicant: Faeth Therapeutics, Inc., San Francisco, CA (US)

(72) Inventors: Oliver D. K. Maddocks, Glasgow (GB); Karen Vousden, Glasgow (GB)

(73) Assignee: Cancer Research Technology Limited, London (GB)

(21) Appl. No.: 17/551,725

(22) Filed: Dec. 15, 2021

Related U.S. Application Data
(60) Provisional application No. 63/170,805, filed on Apr. 5, 2021.
(60) Provisional application No. 63/168,414, filed on Mar. 31, 2021.
(60) Provisional application No. 63/126,294, filed on Dec. 16, 2020.

(51) Int. Cl. A61N 5/10; A61K 31/4245; A61P 35/00

(52) U.S. Cl. CPC A61N 5/1001; A61N 5/1045; A61P 35/00; A61K 31/4245

(57) ABSTRACT

Disclosed herein are formulations and methods of administering formulations to starve cells of nutrients, such as amino acids. The formulations of the present invention can be substantially be devoid of one or more amino acid. The formulations of the present invention can be administered in a combination with an amino acid biosynthesis inhibitor or radiotherapy. A method disclosed herein can sensitize a cell to serine and/or glycine depletion.

### CROSS-REFERENCE

This application claims the benefit of U.S. Provisional Application No. 63/126,294, filed Dec. 16, 2020; U.S. Provisional Application No. 63/168,414, filed Mar. 31, 2021; and U.S. Provisional Application No. 63/170,805, filed Apr. 5, 2021, which are incorporated herein by reference in their entirety.

### SEQUENCE LISTING

The instant application contains a Sequence Listing which has been submitted electronically in ASCII format and is hereby incorporated by reference in its entirety. Said ASCII copy, created on Feb. 4, 2022, is named 57630-709_201_SL.txt and is 4,720 bytes in size.

### BACKGROUND

Many tumor cells show dependence on exogenous serine, and dietary serine and glycine starvation can inhibit the growth of these cancers and extend survival. However, numerous direct and indirect mechanisms promote resistance to this therapeutic approach, including those that promote increased availability of serine (e.g. serine synthesis/serine recycling) or downregulation of pathways that consume serine or glycine.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY OF THE INVENTION

In some embodiments, the invention provides a method of treating a cancer in a subject in need thereof, the method comprising: a) administering to the subject a therapeutically-effective amount of a pharmaceutical composition, wherein the pharmaceutical composition is substantially devoid of at least two amino acids, for a first amount of time; b) a radiation therapy for a second amount of time; and c) after the first amount of time and the second amount of time, waiting a third amount of time, wherein the subject is not administered the pharmaceutical composition or the radiotherapy during the third amount of time.

In some embodiments, the invention provides a method of treating a cancer in a subject in need thereof, the method comprising: a) administering to the subject a therapeutically-effective amount of a pharmaceutical composition, wherein the pharmaceutical composition is substantially devoid of at least two amino acids; and b) an indoleamine 2,3-dioxygenase 1 (IDO1) inhibitor.

In some embodiments, the invention provides a method of treating a cancer in a subject in need thereof, the method comprising: a) administering to the subject a therapeutically-effective amount of a pharmaceutical composition, wherein the pharmaceutical composition is substantially devoid of at least two amino acids; and b) a therapeutically-effective amount of epacadostat.

### BRIEF DESCRIPTION OF THE FIGURES

FIG. 1 depicts the serine synthesis pathway.

FIG. 2 depicts growth curves of the colon cancer cell lines grown in complete medium (CM) or equivalent medium lacking serine and glycine (−SG) and treated or not with 10 μM PH755. Data are presented as mean±SEM of triplicate cultures and are representative of at least two independent experiments (*p<0.05, ** p<0.01, ***p<0.001, **** p<0.0001; two-way ANOVA with Tukey's post hoc test).

FIG. 3 depicts growth curves of the indicated cell lines grown in complete medium (CM) or equivalent medium lacking serine and glycine (−SG) and treated or not with 10 μM PH755. Data are presented as mean±SEM of triplicate cultures and are representative of at least two independent experiments (* p<0.05, ** p<0.01, ***p<0.001, **** p<0.0001; two-way ANOVA with Tukey's post hoc test).

FIG. 4 shows the percentage of BrdU positive cells in HCT116 and DLD-1 cells grown in CM or −SG medium+/−10 μM PH755 for 48 hours followed by a 5 hours incubation with 10 μM BrdU. Data represents mean±SEM of 3 independent experiments (* p<0.05, ** p<0.01, *** p<0.001, one-way ANOVA with Tukey's post hoc test).

FIG. 5 shows the gating strategy to determine the percentage of BrdU positive cells (left panel) and the percentage of cells undergoing different phases of the cell cycle (right panel), taking as an example HCT116 and DLD-1 cells grown in CM and incubated for 30 minutes with 10 μM BrdU.

FIG. 6 shows intracellular serine and glycine levels in HT-29, HCT116, DLD-1, and MDA-MB-468 cells grown in CM or −SG medium+/−10 μM PH755 were measured by LC-MS. Data are presented as mean±SEM of triplicate cultures and are representative of three independent experiments (* p<0.05, ***p<0.001, **** p<0.0001; one-way ANOVA with Tukey's post hoc test).

FIG. 7 shows the percentage of SubG1 cells in HCT116 and DLD-1 cells grown in CM or −SG medium+/−10 μM PH755 for 48 hours. Data represents mean±SEM of 5 independent experiments (** p<0.01, *** p<0.001, one-way ANOVA with Tukey's post hoc test).

FIG. 8 shows cells grown in CM or −SG medium supplemented or not with 10 μM PH755 for 2 days (HCT116) or 3 days (DLD-1). Western blots show the expression of cleaved Caspase-3 and Caspase-3. Membrane was reprobed with vinculin as a loading control. Data are representative of three independent experiments.

FIG. 9 shows the intracellular serine level in HT-29, HCT116, DLD-1, and MDA-MB-468 cells grown in CM or −SG medium+/−10 μM PH755 containing U-[¹³C]-glucose was measured by LC-MS. Metabolite percentages are represented as mean±SEM of triplicate cultures and are representative of three independent experiments (* p<0.05, ** p<0.01, ***p<0.001, **** p<0.0001; one-way ANOVA with Tukey's post hoc test).

FIG. 10 shows intracellular glycine level in HT-29, HCT116, DLD-1 and MDA-MB-468 cells grown in CM or −SG medium+/−10 μM PH755 containing U-[¹³C]-glucose was measured by LC-MS. Metabolite percentages are represented as mean±SEM of triplicate cultures and are representative of three independent experiments (* p<0.05, ** p<0.01; one-way ANOVA with Tukey's post hoc test).

FIG. 11 HT-29 and DLD-1 cells infected with Cas9/PHGDH single guide RNA (sgRNA) were cultured in CM or −SG medium for 24 hours. Western blot shows efficient PHGDH depletion in these cells. Membrane was reprobed with vinculin as a loading control.

FIG. 12 shows growth curves of HT-29 and DLD-1 cells infected with Cas9/PHGDH sgRNA (PHGDH) grown in CM or in −SG medium. Data are presented as mean±SEM of triplicate cultures and are representative of three independent experiments (* p<0.05, ** p<0.01, ***p<0.001, **** p<0.0001; two-way ANOVA with Tukey's post hoc test).

FIG. 13 shows intestinal tumor organoids derived from Vil1-creER;Apcfl/fl (Apc) and Vil1-creER;Apcfl/fl;KrasG12D/+(Apc Kras) mice grown in CM or −SG medium supplemented or not with 10 μM PH755. Left panel: Representative pictures of the organoids are shown before (day 0) and 2 days after medium change. Right panel: Quantification of organoid diameter 2 (Apc) or 4 days (Apc Kras) after medium change. Data are presented as mean±SEM (n=number of organoids measured per condition; Apc: CM: n=113, CM+PH755: n=200, −SG: n=190, −SG+PH755: n=158; Apc Kras: CM: n=149, CM+PH755: n=134, −SG: n=134, −SG+PH755: n=78) and are representative of at least two independent experiments (***p<0.001, **** p<0.0001; one-way ANOVA with Tukey's post hoc test). Scale bar represents 200 μm.

FIG. 14 shows intestinal organoids with Apc truncation (Apc5) or derived from Villin-CreER;Apcfl/fl;KrasG12D/+ mice (Apc Kras 2) grown for 4 days in tumor organoid medium with (CM) or without (−SG) serine and glycine supplemented or not with 10 μM PH755. Representative pictures of the organoids from at least 2 independent experiments are shown before (day 0), 2 days and 4 days after medium change. Scale bar represents 200 μm.

FIG. 15 shows normal organoids derived from the proximal part of healthy small intestine from a Villin-CreERT2 mouse grown in normal organoid medium (containing Wnt-3a) with (CM) or without (−SG) serine and glycine supplemented or not with 10 μM PH755. Representative pictures of the organoids from 3 independent experiments are shown 3 days after medium change. Scale bar represents 200 μm.

FIG. 16 shows four patient-derived colorectal organoids grown in human organoid medium with (CM) or without (−SG) serine and glycine supplemented or not with 10 μM PH755. Representative pictures of the organoids from at least 2 independent experiments are shown 10 to 12 days after medium change. Scale bar represents 200 μm.

FIG. 17 depicts a scheme representing the fate of uniformly carbon labelled glucose (m+6) into purine and glutathione synthesis. Glucose is converted through the pentose phosphate pathway into ribose-5-phosphate, a five-carbon sugar (m+5), that will be added to purine bases to form purine nucleosides. Purine rings also contain two one-carbon units and an intact glycine that can both come from serine metabolism. Serine is synthesized from the glycolytic intermediate 3-PG, producing an m+3 isotopomer from uniformly labelled glucose. Serine (m+3) can generate labelled glycine (m+2) and labelled one-carbon units (m+1). The combination of labelled ribose phosphate, glycine and one-carbon units can thus generate m+5 and greater labelled purines. While m+5 labelled purines represent a contribution of glucose to ribose synthesis alone, m+6-9 labelled purines are likely to represent a contribution from de novo synthesized serine. Glutathione is made from glycine, glutamate (both can be m+2 labelled from glucose) and cysteine. The main isotopomer (m+2) of glutathione is likely to be derived from m+2 glycine with the m+4 labelling reflecting incorporation of m+2 glycine and m+2 glutamate.

FIG. 18 shows intracellular ATP levels in HT-29, HCT116, DLD-1 and MDA-MB-468 cells grown in CM or −SG medium+/−10 μM PH755 containing U-[¹³C]-glucose were measured by LCMS. Metabolite percentages are represented as mean±SEM of triplicate cultures and are representative of three independent experiments. Statistics have been performed comparing the sum of m+6-9% of metabolite pool for ATP and GTP and the sum of m+2-4% of metabolite pool for GSH between experimental groups (* p<0.05, ** p<0.01, ***p<0.001, **** p<0.0001; one-way ANOVA with Tukey's post hoc test).

FIG. 19 shows intracellular glutamate levels in cells grown in CM or −SG medium+/−10 μM PH755 containing U-[¹³C]-glucose were measured by LC-MS. Metabolite percentages are represented as mean±SEM of triplicate wells and are representative of three independent experiments.

FIG. 20 shows intracellular GSH and GTP levels in HT-29, HCT116, DLD-1 and MDA-MB-468 cells grown in CM or −SG medium+/−10 μM PH755 containing U-[¹³C]-glucose were measured by LCMS. Metabolite percentages are represented as mean±SEM of triplicate cultures and are representative of three independent experiments. Statistics have been performed comparing the sum of m+6-9% of metabolite pool for ATP and GTP and the sum of m+2-4% of metabolite pool for GSH between experimental groups (* p<0.05, ** p<0.01, ***p<0.001, **** p<0.0001; one-way ANOVA with Tukey's post hoc test).

FIG. 21 shows intracellular ATP, GTP, and GSH levels in HT-29, HCT116 and DLD-1 cells grown in CM or −SG medium+/−10 μM PH755 containing U-[13C]-glucose for 3 hours or 6 hours were measured by LC-MS. Metabolite percentages are represented as mean±SEM of triplicate cultures and are representative of two independent experiments (* p<0.05, ** p<0.01, ***p<0.001, **** p<0.0001; one-way ANOVA with Tukey's post hoc test).

FIG. 22 shows total levels of ATP, GTP and GSH in cells grown in CM or −SG medium +/−10 μM PH755 were measured by LC-MS. Data are presented as mean±SEM of triplicate cultures and are representative of three independent experiments (* p<0.05, ** p<0.01, ***p<0.001, **** p<0.0001; one-way ANOVA with Tukey's post hoc test).

FIG. 23 depicts a proliferation assay of HT-29 and HCT116 cells grown in −SG medium or −SG medium+10 μM PH755 supplemented or not with 1 mM sodium formate (For), 0.4 mM glycine (Gly) or both (For/Gly). Data are presented as mean±SEM of triplicate cultures and are representative of three independent experiments (* p<0.05, ** p<0.01; two-way ANOVA with Tukey's post hoc test).

FIG. 24 and FIG. 25 show HCT116 cells were grown in −SG medium or −SG medium+10 μM PH755 supplemented or not with 1 mM sodium formate (For), 0.4 mM glycine (Gly) or both (For/Gly) in presence of U-[¹³C]-glucose. ATP and GTP levels were measured by LC-MS. Metabolite percentages are represented as mean±SEM of triplicate cultures and are representative of two independent experiments. Serine level was measured by LC-MS. Data are presented as mean±SEM of triplicate cultures and are representative of two independent experiments (* p<0.05, ** p<0.01, **** p<0.0001; one-way ANOVA with Tukey's post hoc test).

FIG. 26 shows HT-29, HCT116 and DLD-1 cells grown in −SG medium+10 μM PH755 supplemented with 1 mM sodium formate and 0.4 mM glycine for 24 hours in presence of ¹³C₂¹⁵N₁-Glycine for the last hour. ¹³C₂¹⁵N₁-Serine intracellular level was measured by LC-MS after adding a pulse of unlabeled 1 mM serine in the extracellular medium (+serine pulse) or not (−serine pulse) 1 minute before metabolite extraction. Data are presented as mean±SEM of triplicate wells and are representative of three independent experiments.

FIG. 27, FIG. 28, and FIG. 29 show HT-29 and DLD-1 cells infected with Cas9/PHGDH sgRNA (PHGDH) were grown in CM or in −SG medium in presence of U-[¹³C]-glucose. Serine, ATP, GTP, and GSH levels were measured by LC-MS. Data are presented as mean±SEM of triplicate cultures and are representative of two independent experiments (* p<0.05, ** p<0.01, *** p<0.001, **** p<0.0001; (a) unpaired two-tailed Student t test, (b-c) one-way ANOVA with Tukey's post hoc test).

FIG. 30 shows growth curves of HT-29, HCT116 and DLD-1 cells transiently depleted of ATF-4 using short interfering RNA (siRNA) and cultured in −SG medium for 4 days. Data are presented as mean±SEM of triplicate cultures and are representative of two independent experiments (* p<0.05, ** p<0.01, **** p<0.0001; two-way ANOVA with Sidak's post hoc test).

FIG. 31 shows cells grown in CM or −SG medium supplemented or not with 10 μM PH755 for 24 hours. Western blots show the expression of the three SSP enzymes PHGDH, PSAT and PSPH (membrane was reprobed with vinculin as a loading control) or the expression of the ATF-4 target ASNS (membrane was reprobed with vinculin as a loading control). Data are representative of at least two independent experiments.

FIG. 32 shows HT-29 and DLD-1 cells infected with Cas9/PHGDH single guide RNA (sgRNA) were cultured in CM or −SG medium for 24 hours. Western blots show expression of PHGDH, PSAT and PSPH (membrane was reprobed with vinculin as a loading control) or expression of ATF-4 and ASNS (membrane was reprobed with vinculin as a loading control) in these cells. Data are representative of three independent experiments.

FIG. 33 shows HT-29 and HCT116 cells grown in CM or −SG medium supplemented or not with 10 μM PH755 for 4 h, 8 h, 12 h, 16 h, and 24 h. Western blots show SSP enzymes expression in these cells. Each membrane was reprobed with vinculin as a loading control. Data are representative of two independent experiments.

FIG. 34 shows HCT116 and DLD-1 cells grown in CM or −SG medium supplemented or not with 10 μM PH755 for 24 hours. Western blots show Phospho-GCN2 (Thr899), GCN2, Phospho-eIF2a (Ser51) and eIF2a. Membranes were reprobed with vinculin as a loading control. Data are representative of two independent experiments.

FIG. 35 shows HCT116 and DLD-1 cells grown in CM or −SG medium supplemented or not with 10 μM PH755 for 24 hours. When indicated, cells were treated with 10 μM MG-132, a proteasome inhibitor, 6 hours before harvesting the cells. Western blots show the expression of ATF-4 and its targets ASNS and PSAT. Membrane was reprobed with vinculin as a loading control. Data are representative of three independent experiments.

FIG. 36 shows HT-29, HCT116 and DLD-1 cells grown in CM or −SG medium supplemented or not with 10 μM PH755 for 6 hours or 24 hours. Relative gene expression of ATF4 and PHGDH were measured by qPCR and normalized to the cells grown in CM for 6 hours. Data are presented as mean±SEM of triplicate cultures and are representative of two independent experiments (* p<0.05, ** p<0.01, ***p<0.001, **** p<0.0001; one-way ANOVA with Tukey's post hoc test).

FIG. 37 shows HT-29, HCT116 and DLD-1 cells grown in CM or −SG medium supplemented or not with 10 μM PH755 for 6 hours or 24 hours. Relative gene expression of ASNS, PSAT1 and PSPH were measured by qPCR and normalized to the cells grown in CM for 6 hours. Data are presented as mean±SEM of triplicate cultures and are representative of two independent experiments (* p<0.05, ** p<0.01, ***p<0.001, **** p<0.0001; one-way ANOVA with Tukey's post hoc test).

FIG. 38 shows HT-29, HCT116 and DLD-1 cells grown in CM, −SG medium or −SG medium+10 μM PH755 supplemented or not with 1 mM sodium formate plus 0.4 mM glycine (For/Gly). Western blot shows the expression of the three SSP enzymes PHGDH, PSAT and PSPH or the expression of ATF-4 and its canonical target ASNS after a 24 hours incubation in these medium. Membrane was reprobed with vinculin as a loading control. Data are representative of two independent experiments.

FIG. 39 shows HCT116 and DLD-1 cells grown in CM or −SG medium supplemented or not with 10 μM PH755 for 24 hours. Puromycin (90 μM) was added in culture medium 10 minutes before harvesting the cells. When indicated, cells were treated with 10 μg/mL cycloheximide (CHX), a well-known protein synthesis inhibitor, 5 hours before harvesting the cells. Western blots show puromycylated peptides. Membrane was reprobed with vinculin as a loading control. Data are representative of two independent experiments.

FIG. 40 shows HCT116 and DLD-1 cells grown in CM or −SG medium supplemented or not with 10 μM PH755 for 24 hours. When indicated, cells were treated with 10 μM MG-132, a proteasome inhibitor, 6 hours before harvesting the cells. Western blots show the expression of c-MYC, HIF1a and p53. Membrane was reprobed with vinculin as a loading control. Data are representative of three independent experiments.

FIG. 41 shows DLD-1 and HT-29 cells grown in CM or −SG medium supplemented or not with 10 μM PH755 for 24 hours. Western blots show Phospho-p70S6K (Thr389) and p70S6K. Membrane was reprobed with actin as a loading control. Data are representative of three independent experiments.

FIG. 42 shows HT-29, HCT116, and DLD-1 cells grown in CM for 24 hours. When indicated, cells were treated with the ER stress inducer, tunicamycin (5 μg/mL) or 10 μM PH755. Western blots show the expression of the ATF-4 targets ASNS, PHGDH, PSAT and PSPH. Membrane was reprobed with vinculin as a loading control. Data are representative of two independent experiments.

FIG. 43 shows HT-29, HCT116, and DLD-1 cells grown in CM, −SG medium, or −SG medium+10 μM PH755 supplemented or not with 1 mM sodium formate plus 0.4 mM glycine (For/Gly). Western blot shows the expression of the three SSP enzymes PHGDH, PSAT and PSPH or the expression of ATF-4 and its canonical target ASNS after a 24 hours incubation in these medium. Membrane was reprobed with vinculin as a loading control. Data are representative of two independent experiments.

FIG. 44 shows the weight of C57BL/6J mice fed a control diet (CTR) or an equivalent diet lacking serine and glycine (−SG) and treated with vehicle (Veh) or PH755 was recorded at regular intervals. Percentage of body weight was calculated from the initial weight taken the day of the diet change. Arrows show the starting day of the indicated treatment. Data are presented as mean±SEM (n=10 mice per group). (** p<0.01, **** p<0.0001; two-way ANOVA with Tukey's post hoc test).

FIG. 45 shows low-power magnifications of transverse sections obtained at the level of the caudal diencephalon and rostral mesencephalon from C57BL/6J mice fed a control diet (CTR) or an equivalent diet lacking serine and glycine (−SG) and treated with vehicle (Veh) or PH755 (n=5 mice per group). There is no evidence of degeneration or malacia on hematoxylin and eosin stained sections. Brain weight for each experimental group of mice is shown as mean±SEM (n=5 mice/group).

FIG. 46 shows high-power magnifications of transverse sections obtained at the level of the cerebral cortex from C57BL/6J mice fed a control diet (CTR) or an equivalent diet lacking serine and glycine (−SG) and treated with vehicle (Veh) or PH755 (n=5 mice per group). Neurons and neuropil are morphologically unremarkable. Scale bar represents 50 μm.

FIG. 47 shows plasma that was taken at time of sacrifice from C57BL/6J fed a control diet (CTR) or an equivalent diet lacking serine and glycine (−SG) and treated with vehicle (Veh) or PH755 for 20 days. AST and ALT activities in plasma were measured with commercial kits. Data are presented as mean±SEM (n=10 mice per group).

FIG. 48 shows plasma that was taken at time of sacrifice from C57BL/6J mice fed a control diet (CTR) or an equivalent diet lacking serine and glycine (−SG) and treated with vehicle (Veh) or PH755 for 20 days. LC-MS analysis was performed to evaluate urea and creatinine content. Data are presented as mean±SEM (n=10 mice per group).

FIG. 49 shows quantification of villus length from C57BL/6J mice fed a control diet (CTR) or an equivalent diet lacking serine and glycine (−SG) and treated with vehicle (Veh) or PH755 for 20 days. Data are presented as mean±SEM (n=10 mice per group). (* p<0.05, **** p<0.0001; one way ANOVA with Tukey's post hoc test).

FIG. 50 shows representative images of Ki67-stained jejunum from C57BL/6J mice fed a control diet (CTR) or an equivalent diet lacking serine and glycine (−SG) and treated with vehicle (Veh) or PH755 for 20 days (n=5 mice per group).

FIG. 51 and FIG. 52 show the weight of mice used in the DLD-1 and HCT116 xenograft experiments was recorded at regular intervals. Percentage of body weight was calculated from the initial weight taken the day of the diet change. Arrows show the starting day of the indicated treatment. Data are presented as mean±SEM. (ns: no significance, *p<0.05; ***p<0.001; two-way ANOVA plus Tukey's post hoc test). (a) CTR+Veh: n=10; CTR+PH755 n=10; −SG+Veh: n=10; −SG+PH755 n=9. (b) CTR+Veh: n=8; CTR+PH755 n=7; −SG+Veh: n=8; −SG+PH755 n=7 (n=number of mice).

FIG. 53 shows plasma that was taken at time of sacrifice from mice subcutaneously injected with DLD-1 cells, fed a control diet (CTR) or an equivalent diet lacking serine and glycine (−SG) and treated with vehicle (Veh) or PH755. LC-MS analysis was performed to measure absolute levels of serine and glycine in plasma. CTR+Veh: n=10; CTR+PH755 n=10; −SG+Veh: n=10; −SG+PH755 n=9 (n=number of mice). Data are presented as mean±SEM. (** p<0.01, **** p<0.0001, unpaired two-tailed Student t test).

FIG. 54 shows plasma that was taken at time of sacrifice from mice subcutaneously injected with HCT116 cells fed a control diet (CTR) or an equivalent diet lacking serine and glycine (−SG) and treated with vehicle (Veh) or PH755. LC-MS analysis was performed to evaluate serine and glycine content. Data are presented as mean±SEM (n=8 mice per group). (* p<0.05; unpaired two-tailed Student t test).

FIG. 55 shows CD-1 nude mice that were subcutaneously injected with DLD-1 cells and fed a diet with (CTR) or without serine and glycine (−SG) two days after tumor cell injection. Two days after diet change, mice were dosed orally with vehicle (Veh) or PH755 once daily for 20 days. The starting dosage of PH755 was 100 mg/kg (for 7 days) and was subsequently lowered to 50 mg/kg (for 6 days) and increased again to 75 mg/kg (for 7 days). Tumor volumes were measured three times a week by caliper measurement. Data are presented as mean±SEM. CTR+Veh: n=10; CTR+PH755 n=10; −SG+Veh: n=10; −SG+PH755 n=9 (n=number of mice). (ns: no significance, **P<0.01; two-way ANOVA plus Tukey's post hoc test).

FIG. 56 shows CD-1 nude mice that were subcutaneously injected with HCT116 cells and fed a diet with (CTR) or without serine and glycine (−SG) ten days after tumor cell injection. Four days after diet change, mice were dosed orally with vehicle (Veh) or PH755 once daily for 11 days. The starting dosage of PH755 was 100 mg/kg (for 3 days) and was subsequently lowered to 50 mg/kg (for 8 days). Tumor volumes were measured three times a week by caliper measurement. Data are presented as mean±SEM. CTR+Veh: n=8; CTR+PH755 n=7; −SG+Veh: n=8; −SG+PH755 n=7 (n=number of mice). (ns: no significance, *P<0.05; two-way ANOVA plus Tukey's post hoc test).

FIG. 57 provides representative immunohistochemistry pictures and quantification of active Caspase-3 positive cells in DLD-1 tumors harvested at end-points from mice fed a control diet (CTR) or an equivalent diet lacking serine and glycine (−SG) and treated with vehicle (Veh) or PH755. CTR+Veh: n=9; CTR+PH755 n=9; −SG+Veh: n=10; −SG+PH755 n=8 (n=number of mice). Data are presented as mean±SEM. (*P<0.05; unpaired two-tailed Student t test with Welch's correction). Scale bar represents 50 μm.

FIG. 58 and FIG. 61 show serine, glycine, SAM and SAH levels measured by LC-MS in tumor lysates collected at endpoint from animals subcutaneously injected with DLD-1 cells. Peak area was normalized to total ion count (TIC). (e) CTR+Veh: n=10; CTR+PH755 n=10; −SG+Veh: n=10; −SG+PH755 n=9. (f) CTR+Veh: n=9; CTR+PH755 n=9; −SG+Veh: n=10; −SG+PH755 n=8 (n=number of mice) (* p<0.05, ** p<0.01; unpaired two-tailed Student t test with Welch's correction applied when necessary).

FIG. 59 shows serine and glycine levels measured by LC-MS in tumor lysates collected at end-point from animals subcutaneously injected with HCT116 cells. Peak area was normalized to total ion count (TIC). CTR+Veh: n=8; CTR+PH755 n=6; −SG+Veh: n=8; −SG+PH755 n=8 (n=number of mice). Data are presented as mean±SEM. (* p<0.05, ** p<0.01; unpaired two-tailed Student t test).

FIG. 60 shows ATP and GTP levels measured by LC-MS in tumor lysates collected at end-point from animals subcutaneously injected with DLD-1 cells. Peak area was normalized to total ion count (TIC). CTR+Veh: n=10; CTR+PH755 n=10; −SG+Veh: n=10; −SG+PH755 n=9 (n=number of mice). Data are presented as mean±SEM. (ns: no significance; unpaired two-tailed Student t test).

FIG. 62 and FIG. 63 show representative immunohistochemistry pictures and quantification of PHGDH staining and PSAT staining in DLD-1 tumors harvested at end-points from mice fed a control diet (CTR) or an equivalent diet lacking serine and glycine (−SG) and treated with vehicle (Veh) or PH755. CTR+Veh: n=9; CTR+PH755 n=9; −SG+Veh: n=9; −SG+PH755 n=7 (n=number of mice). Data are presented as mean±SEM. (* p<0.05, ***p<0.001; one-way ANOVA with Tukey's post hoc test). Scale bar represents 50 μm.

FIG. 64 PANEL A-PANEL E show how combining dietary restriction of serine and glycine and PHGDH inhibition cooperate to lower tumor burden and improve survival in genetic models of intestinal cancer.

FIG. 65 PANEL A-PANEL D show the metabolomic impact of radiation on pancreatic and colorectal cancer cells in vitro.

FIG. 66 PANEL A-PANEL E show the effect of dietary amino acid restriction in response to targeted radiotherapy in vivo.

FIG. 67 shows IDO1 expression in vivo. PANEL A is a schematic detailing the methods used to analyze IDO1 expression in genetically engineered mouse models (GEMM) of pancreatic ductal adenocarcinoma (PDAC). PANEL B shows the indicated proteins after analysis by immunoblotting. PANEL C shows the quantification of IDO1 relative to total protein (load control) (healthy pancreas n=5, Pdx1-cre;Kras^{G12D/+};Trp53^{fl/+} tumors n=6, Pdx1-cre;Kras^{G12D/+};Trp53^{R172H/+} tumors n=5, unpaired t-tests, p values shown, error bars are std. dev.). PANEL D shows KPC A cells, a line isolated from tumors of mixed-background Pdx1-cre;Kras^{G12D/+};Trp53^{R172H/+} mice were treated either with mouse IFNγ (1 ng/ml) for 24 h, or subcutaneously injected into the flank of CD1-nude mice to form tumors. PANEL E shows KPC cells were isolated from pure C567Bl6/J background Pdx1-cre;Kras^{G12D/+};Trp53^{R172H/+} mice and either treated in culture with mouse IFNγ (1 ng/mL) for 24 h or subcutaneously injected into the flank of C567Bl6/J mice to form tumors. PANEL F shows the indicated cell lines were treated with human IFNγ (1 ng/ml) for 24 h and cell lysates blotted for the indicated proteins.

FIG. 68 shows data extracted from the MERAV database showing the relative abundance of IDO1 mRNA from microarrays.

FIG. 69 shows that IDO expression was upregulated by ultra-low-attachment tissue culture plate (3D) growth of cells and proteins. and IFNγ via JAK/STAT signaling. PANEL A shows a schematic detailing the kynurenine pathway through which tryptophan is metabolized. PANEL B shows the expression of proteins cultured under either normoxic (20% 02) or hypoxic (1% 02) conditions. PANEL C shows the expression of proteins treated with rotenone (1 μM) or vehicle only control. PANEL D shows the expression of proteins cultured in media containing either glucose (Glc) (10 mM) or galactose (Gal) (10 mM). PANEL E shows proteins cultured in 2D or 3D conditions. PANEL F shows the fluorescence intensities of IDO1/Actin for CFPAC-1 in 2D and 3D conditions, quantified (n=4, paired t-test, p value shown, error bars are S.E.M.). PANEL G shows the results of CFPAC-1 cells cultured in 2D or 3D conditions for 24 h and treated with epacadostat (1 μM) or vehicle only control for 16 h before media kynurenine was analyzed by LCMS (1 ex, triplicate wells, error bars are std. dev.). PANEL H shows CFPAC-1 or HPAF-II cells cultured in either 2D or 3D conditions for 24 h and then treated for 16 h with JAKi (1 μM) or vehicle only control (veh.) and/or human IFNγ (1 ng/ml). Cells were then lysed and indicated proteins analyzed by immunoblotting.

FIG. 70 shows CFPAC-1 or HPAF-II cells grown in normal tissue culture plates (2D) or in ultra-low-attachment tissue culture plates (3D) for 24 h, or cultured in 2D and treated with 1 ng/ml IFNγ. Lysates were (PANEL A) blotted for the indicated proteins and (PANEL B) fluorescence intensity of IDO1 relative to actin (load control) was quantified (n=4, paired t-test, p value shown, error bars are S.E.M.). CFPAC-1 and HPAF-II cells were grown in either normal tissue culture plates (2D) or ultra-low-attachment tissue culture plates (3D) for 24 hours, and cell lysates immunoblotted for the indicated proteins after 16 h treatment with MG132 (20 μM) or vehicle-only control (PANEL C); after treatment for the indicated times with bafilomycin A1 (100 nM) or vehicle-only control or (PANEL D); or after 16 h treatment with JAKi (at indicated concentrations), vehicle-only control or IFNγ (1 ng/ml) (PANEL E).

FIG. 71 shows that tryptophan-derived one carbon units are incorporated into serine and nucleotides in pancreatic cancer cells.

FIG. 72 shows CFPAC-1 cells cultured in 2D or 3D for 24 h, then treated for 24 h with epacadostat (1 μM) or vehicle only control in the presence of either unlabeled (¹²C) or ¹³C₁₁ tryptophan and intracellular quantities of the indicated nucleotides were analyzed by LCMS (1 ex, triplicate wells, error bars are std. dev.)

FIG. 73 shows that tryptophan-derived one carbon units are utilized for serine and nucleotide synthesis in PDAC tumors in vivo.

FIG. 74 shows data from KPC cells from pure C57BL/J Pdx1-cre;Kras^{G12D/+};Trp53^{R172H/+} mice expressing IDO1 or empty-vector control (EV). The KPC cells were injected subcutaneously into the flanks of C57BL/J mice; once tumors had formed the mice were given 8004, of 120 mM ¹³C₁₁ tryptophan by intraperitoneal injection and left for 3 h.

FIG. 75 shows that cancer cells released tryptophan-derived formate, which was consumed by pancreatic stellate cells and incorporated into nucleotides. CFPAC-1 (PANEL A) or HPAF-II (PANEL B) cells were cultured in 3D for 4 days and then treated with IFNγ (1 ng/ml) or vehicle only control in the presence of either unlabeled (¹²C), ¹³C₁₁ tryptophan, or ¹³C₃¹⁵N₁ serine for 24 h. Media quantities of formate were analyzed by derivatization and GC-MS (1 ex, triplicate wells, error bars are std. dev.). PANEL C shows a schematic of the experimental approaches used in PANEL D-PANEL K. CFPAC-1 cells were treated with vehicle only control or human IFNγ (1 ng/ml) and epacadostat (epac., 1 μM) or vehicle only control in the presence of unlabeled (¹²C) or ¹³C₁₁ tryptophan. Conditioned media was collected after 24 h and ImPSC's were cultured in this media, or in non-conditioned treatment-matched media. After 24 h, intracellular quantities of serine (PANEL D), ATP (PANEL E), ADP (PANEL F) and AMP (PANEL G) were analyzed by LCMS (fraction of major isotopologues relative to total are shown, 1 ex, triplicate wells, error bars are std. dev.). ImPSC-GFP cells were cultured for 24 h in 2D as a monoculture or in co-culture with CFPAC-1 cells. Cells were then treated with vehicle only control or human IFNγ (1 ng/ml) and epacadostat (1 μM) or vehicle only control in the presence of ¹³C₁₁ tryptophan for 24 h. Cells were then trypsinised and sorted using FACS for GFP-positive cells and intracellular quantities of serine (PANEL H), ATP (PANEL I), ADP (PANEL J) and AMP (PANEL K) were analyzed by LCMS (fraction of major isotopologues relative to total are shown, 1 ex, triplicate wells, error bars are std. dev.). PANEL L shows a proposed model for the use of tryptophan-derived formate in pancreatic ductal adenocarcinoma (PDAC) cells and pancreatic stellate cells.

FIG. 76 shows intracellular uptake of ¹³C₁ formate in ATP, DP, AMP, and GTP in ImPSC #1, ImPSC #2, and ImPSC #3 cells. ImPSC #1, ImPSC #2 & ImPSC #3 cells were cultured for 24 h in the presence of ¹³C₁ formate and intracellular quantities of ATP (PANEL A), ADP (PANEL B), AMP (PANEL C) and GTP (PANEL D), all possible destination for formate-derived one carbons were analyzed by LCMS (1 ex, triplicate wells, error bars are std. dev.). CFPAC-1 cells were treated with IFNγ (1 ng/ml) and/or epacadostat (1 μM) and/or vehicle only controls in the presence of unlabeled (¹²C) or ¹³C₁₁ tryptophan. Conditioned media was collected after 24 h and ImPSC #2 cells were cultured in this media, or in non-conditioned treatment-matched media. After 24 h, intracellular quantities of ATP (PANEL E), ADP (PANEL F) and serine (PANEL G) were analyzed by LCMS (fraction of major isotopologues relative to total are shown 1 ex, triplicate wells, error bars are std. dev.).

FIG. 77 LEFT PANEL shows cell proliferation over 5 days in cells treated with: 1) control+vehicle; 2) −Serine+vehicle; 3) control+epacadostat (1 μM); or 4) −Serine+epacadostat (1 μM). FIG. 77 RIGHT PANEL shows fold changes in cell number at day 5 compared to day 0 in cells treated with: 1) control+vehicle; 2) −Serine+vehicle; 3) control+epacadostat (1 μM); or 4) −Serine+epacadostat (1 μM).

FIG. 78 shows the labelled fractions derived from carbon-13 in cells of AMP, ADP, ATP, GDP, and GMP in cells treated with: 1) control+vehicle; 2) −Serine+vehicle; 3) control+epacadostat (1 μM); or 4) −Serine+epacadostat (1 μM).

### DETAILED DESCRIPTION

Direct mechanisms of promoting resistance to the therapeutic approach of reducing the availability of serine and/or glycine include those that promote increased availability of serine e.g. by serine biosynthesis (at tumor or systemic level) via enhanced expression of the de novo serine synthesis pathway (SSP) enzymes, whose expression can also be promoted by certain oncogenic mutations. Another route for increasing serine availability is the promotion of serine recycling e.g. by mechanisms such as authophagy. Indirect mechanisms of resistance can rely on metabolic adaptations beyond the metabolic pathways directly involved in serine synthesis, for example downregulating pathways (such as nucleotide synthesis) which consume serine. Combination with other therapeutic agents that target these direct or indirect mechanisms of resistance can improve the ability of serine and glycine starvation to inhibit, for example, tumor growth, tumor initiation, or metastasis. Furthermore, combination with therapeutic agents or interventions which increase the demands of a cancer cell or a tumor for serine and/or glycine can also sensitize the cancer cell or tumor to starvation of serine and/or glycine.

Described herein are compositions and method for the inhibition of Phosphoglycerate Dehydrogenase (PHGDH), the first step in the SSP, in combination with compositions devoid of serine and/or glycine. PHGDH cooperates with serine and glycine depletion to inhibit one-carbon metabolism and cancer growth. In vitro, inhibition of PHGDH combined with serine starvation can lead to a defect in global protein synthesis, which can block the activation of an ATF-4 response and more broadly impacts the protective stress response to amino acid depletion. In vivo, the combination of diet and inhibitor can show a therapeutic efficacy against tumors that are resistant to diet or drug alone, along with reduced one-carbon availability. Inhibition of PHGDH can augment the therapeutic efficacy of a serine depleted diet.

Also described herein are methods of administering serine and glycine depletion therapy in combination with a radiotherapy.

Cancer cells can adapt their metabolism to support growth and survival, leading to various dependencies and vulnerabilities that could be targeted for therapy. While these metabolic alterations can be directed by numerous factors, including the genetic alterations in the tumor and the tumor environment or tissue of origin, serine metabolism in supporting cancer cell growth could also be important for these observed metabolic alterations. Serine and glycine (which is produced from serine by the SHMT1/2 reaction) contribute to a number of important processes, including protein, nucleotide, and lipid synthesis, the generation of antioxidant defense through glutathione and NADPH synthesis and the provision of one-carbon units for the folate cycle and methylation reactions.

As a non-essential amino acid, serine can be taken up from the extracellular environment or synthesized de novo by cells using the serine synthesis pathway (SSP). Cancer cells can avidly consume serine and depend on an exogenous source of serine for optimal growth. Some cancer cells can adapt to serine starvation by activating flux through the SSP. Serine is an activator of PKM2, the final step in glycolysis, and decreased PKM2 activity under serine depleted conditions can allow for the diversion of glycolytic intermediates into the SSP. This response is coordinated with an ATF-4 and histone methyltransferase G9A-dependent activation of the three enzymes of the SSP, which can allow most cancer cells to survive and continue to proliferate following serine starvation. The efficacy with which cancer cells can adapt to the loss of exogenous serine depends on several factors. Some cancers acquire an amplification or overexpression of PHGDH—the first step in the SSP—and these cells tend to be less affected by serine starvation. Similarly, activation of oncogenes such as KRAS, MYC, MDM2, and NRF210 can lead to an increase in SSP enzyme expression, also allowing cells to become resistant to depletion of exogenous serine. Conversely, although the p53 tumor suppressor protein can inhibit PHGDH expression, loss of p53 also makes cells more vulnerable to increased ROS that accompanies the switch to de novo serine synthesis, resulting in a decreased survival in serine free medium.

### Amino Acids

A composition disclosed herein can lack serine. A composition disclosed herein can lack glycine. A composition disclosed herein can lack serine and glycine. A composition disclosed herein can be administered in combination with a PHGDH inhibitor, PSAT1 inhibitor, or a PSPH inhibitor.

A composition of the disclosure comprises at least ten amino acids or salts thereof. In some embodiments, a composition of the disclosure comprises 10, 11, 12, 13, 14, 15, 16, 17, 18, or 19 amino acids or a salt thereof. In some embodiments, a composition of the disclosure comprises 10 amino acids or a salt thereof. In some embodiments, a composition of the disclosure comprises 14 amino acids or a salt thereof. In some embodiments, a composition of the disclosure comprises 18 amino acids or a salt thereof. A salt of an amino acid disclosed herein can be a pharmaceutically acceptable salt. In some embodiments, a composition disclosed herein is devoid of serine and glycine. In some embodiments, a composition disclosed herein is devoid of serine. In some embodiments, a composition disclosed herein is devoid of glycine.

In some embodiments, a composition of the disclosure comprises 1, 2, 3, 4, 5, 6, 7, 8, or 9 essential amino acids or salts thereof. In some embodiments, a composition of the disclosure comprises 7, 8, or 9 essential amino acids or salts thereof. In some embodiments, a composition of the disclosure comprises 8 essential amino acids or salts thereof. In some embodiments, a composition of the disclosure comprises 9 essential amino acids or salts thereof. A salt of an amino acid disclosed herein can be a pharmaceutically acceptable salt. In some embodiments, a composition disclosed herein is devoid of serine and glycine. In some embodiments, a composition disclosed herein is devoid of serine. In some embodiments, a composition disclosed herein is devoid of glycine.

In some embodiments, a composition of the disclosure comprises 1, 2, 3, 4, 5, 6, 7, 8, 9, 10, or 11 non-essential amino acids or salts thereof. In some embodiments, a composition of the disclosure comprises 7, 8, 9, 10, or 11 non-essential amino acids or salts thereof. In some embodiments, a composition of the disclosure comprises 7 non-essential amino acids or salts thereof. In some embodiments, a composition of the disclosure comprises 8 non-essential amino acids or salts thereof. In some embodiments, a composition of the disclosure comprises 9 non-essential amino acids or salts thereof. A salt of an amino acid disclosed herein can be a pharmaceutically acceptable salt. In some embodiments, a composition disclosed herein is devoid of serine and glycine. In some embodiments, a composition disclosed herein is devoid of serine. In some embodiments, a composition disclosed herein is devoid of glycine.

A composition of the disclosure can comprise essential amino acids or salts thereof and non-essential amino acids or salts thereof. In some embodiments, a composition of the disclosure comprises 1, 2, 3, 4, 5, 6, 7, 8, or 9 essential amino acids or salts thereof and 1, 2, 3, 4, 5, 6, 7, 8, 9, 10, or 11 non-essential amino acids or salts thereof. In some embodiments, a composition of the disclosure comprises 7, 8, or 9 essential amino acids or salts thereof and 6, 7, 8, or 9 non-essential amino acids or salts thereof. In some embodiments, a composition of the disclosure comprises 8 or 9 essential amino acids or salts thereof and 8 or 9 non-essential amino acids or salts thereof. In some embodiments, a composition of the disclosure comprises 9 essential amino acids or salts thereof and 7 non-essential amino acids or salts thereof. In some embodiments, a composition of the disclosure comprises 9 essential amino acids or salts thereof and 8 non-essential amino acids or salts thereof. In some embodiments, a composition of the disclosure comprises 9 essential amino acids or salts thereof and 9 non-essential amino acids or salts thereof. A salt of an amino acid disclosed herein can be a pharmaceutically acceptable salt. In some embodiments, a composition disclosed herein is devoid of serine and glycine. In some embodiments, a composition disclosed herein is devoid of serine. In some embodiments, a composition disclosed herein is devoid of glycine.

In some embodiments, a composition of the disclosure comprises histidine, isoleucine, leucine, lysine, methionine, cysteine, phenylalanine, tyrosine, threonine, tryptophan, valine, arginine, glutamine, alanine, aspartic acid, asparagine, glutamic acid or proline. In some embodiments, a composition of the disclosure comprises L-histidine, L-isoleucine, L-leucine, L-lysine, L-methionine, L-cysteine, L-phenylalanine, L-tyrosine, L-threonine, L-tryptophan, L-valine, L-arginine, L-glutamine, L-alanine, L-aspartic acid, L-asparagine, L-glutamic acid, or L-proline.

In some embodiments, a composition comprises histidine or a salt thereof, such as L-histidine or L-histidine hydrochloride. In some embodiments, a composition of the disclosure comprises isoleucine or a salt thereof, such as L-isoleucine, L-isoleucine methyl ester hydrochloride, or L-isoleucine ethyl ester hydrochloride. A salt of an amino acid disclosed herein can be a pharmaceutically acceptable salt. In some embodiments, a composition of the disclosure comprises leucine or a salt thereof, such as L-leucine, L-leucine methyl ester hydrochloride, or L-leucine ethyl ester hydrochloride. A salt of an amino acid disclosed herein can be a pharmaceutically acceptable salt. In some embodiments, a composition of the disclosure comprises lysine or a salt thereof, such as L-lysine, L-lysine hydrochloride, or L-lysine dihydrochloride. A salt of an amino acid disclosed herein can be a pharmaceutically acceptable salt. In some embodiments, a composition of the disclosure comprises methionine or a salt thereof, such as L-methionine, L-methionine methyl ester hydrochloride, or L-methionine hydrochloride. A salt of an amino acid disclosed herein can be a pharmaceutically acceptable salt.

In some embodiments, a composition of the disclosure comprises cysteine or a salt thereof, such as L-cysteine, L-cysteine hydrochloride, L-cysteine methyl ester hydrochloride, or L-cysteine ethyl ester hydrochloride. In some embodiments, a composition discloses cystine or a salt thereof, such as L-cystine. A salt of an amino acid disclosed herein can be a pharmaceutically acceptable salt. In some embodiments, a composition of the disclosure comprises phenylalanine or a salt thereof, such as L-phenylalanine, DL-phenylalanine, or L-phenylalanine methyl ester hydrochloride. In some embodiments, a composition of the disclosure comprises tyrosine or a salt thereof, such as L-tyrosine or L-tyrosine hydrochloride. In some embodiments, a composition of the disclosure comprises threonine or a salt thereof, such as L-threonine or L-threonine methyl ester hydrochloride. In some embodiments, a composition of the disclosure comprises L-tryptophan. In some embodiments, a composition of the disclosure comprises valine or a salt thereof, such as L-valine, L-valine methyl ester hydrochloride, or L-valine ethyl ester hydrochloride. A salt of an amino acid disclosed herein can be a pharmaceutically acceptable salt.

In some embodiments, a composition of the disclosure comprises arginine or a salt thereof, such as L-arginine or L-arginine hydrochloride. In some embodiments, a composition of the disclosure comprises glutamine or a salt thereof, such as L-glutamine or L-glutamine hydrochloride. In some embodiments, a composition of the disclosure comprises alanine or a salt thereof, such as L-alanine or β-alanine. In some embodiments, a composition of the disclosure comprises aspartic acid or a salt thereof, such as L-aspartic acid, D-aspartic acid, L- or D-aspartic acid potassium salt, L- or D-aspartic acid hydrochloride salt; L- or D-aspartic acid magnesium salt, or L- or D-aspartic acid calcium salt. In some embodiments, a composition of the disclosure comprises L-asparagine. In some embodiments, a composition of the disclosure comprises glutamic acid or a salt thereof, such as L-glutamic acid or L-glutamic acid hydrochloride. In some embodiments, a composition of the disclosure comprises proline or a salt thereof, such as L-proline, L-proline hydrochloride, L-proline methyl ester hydrochloride, or L-proline ethyl ester hydrochloride. A salt of an amino acid disclosed herein can be a pharmaceutically acceptable salt.

### Pharmaceutical Excipients

A composition of the disclosure can comprise at least one pharmaceutical excipient, such as an anti-adherent, a binder, coating, colorant, disintegrant, flavorant, preservative, sorbent, sweetener, or vehicle. In some embodiment, a composition of the disclosure comprises a colorant and a flavorant. In some embodiment, a composition of the disclosure comprises a colorant, flavorant, and sweetener. In some embodiment, a composition of the disclosure comprises a flavorant, sweetener, and a preservative.

### Formulations

A composition of the invention can be, for example, an immediate release form or a controlled release formulation. An immediate release formulation can be formulated to allow the compounds to act rapidly. Non-limiting examples of immediate release formulations include readily dissolvable formulations. A controlled release formulation can be a pharmaceutical formulation that has been adapted such that release rates and release profiles of the active agent can be matched to physiological and chronotherapeutic requirements or, alternatively, has been formulated to effect release of an active agent at a programmed rate. Non-limiting examples of controlled release formulations include granules, delayed release granules, hydrogels (e.g., of synthetic or natural origin), other gelling agents (e.g., gel-forming dietary fibers), matrix-based formulations (e.g., formulations comprising a polymeric material having at least one active ingredient dispersed through), granules within a matrix, polymeric mixtures, and granular masses.

In some embodiments, a controlled release formulation is a delayed release form. A delayed release form can be formulated to delay a compound's action for an extended period of time. A delayed release form can be formulated to delay the release of an effective dose of one or more compounds, for example, for about 4, about 8, about 12, about 16, or about 24 hours.

A controlled release formulation can be a sustained release form. A sustained release form can be formulated to sustain, for example, the compound's action over an extended period of time. A sustained release form can be formulated to provide an effective dose of any compound described herein (e.g., provide a physiologically-effective blood profile) over about 4, about 8, about 12, about 16, or about 24 hours.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

### Dosing

A composition described herein can be given to supplement a meal consumed by a subject. A composition described herein can be given as a meal replacement. A composition described herein can be given immediately before or immediately after a meal. A composition described here can be given within about 5 minutes, about 10 minutes, about 15 minutes, about 20 minutes, about 25 minutes, about 30 minutes, about 40 minutes, about one hour, about 2 hours, about 3 hours, about 4 hours, about 5 hours, or about 6 hours before or after a meal.

A composition described herein can be in unit dosage forms suitable for single administration of precise dosages. In unit dosage form, the formulation is divided into unit doses containing appropriate quantities of the composition. In some embodiments, the unit dosage can be in the form of a package containing discrete quantities of the formulation. In some embodiments, formulations of the disclosure can be presented in unit dosage form in single-serving sachet. In some embodiments, formulations of the disclosure can be presented in a single-dose non-reclosable container. In some embodiments, a formulation of the disclosure can be presented in a reclosable container, and the subject can obtain a single-dose serving of the formulation using a scoop or spoon designed to distribute a single-dose serving. In some embodiments, a formulation of the disclosure can be presented in a reclosable container, and the subject can obtain a single-dose serving of the formulation using a scoop or spoon designed to distribute a half-dose serving (i.e., two scoops to distribute one serving).

A composition described herein can be present in a unit dose serving in a range from about 1 g to about 2 g, from about 2 g to about 3 g, from about 3 g to about 4 g, from about 4 g to about 5 g, from about 5 g to about 6 g, from about 6 g to about 7 g, from about 7 g to about 8 g, from about 8 g to about 9 g, from about 9 g to about 10 g, from about 10 g to about 11 g, from about 11 g to about 12 g, from about 12 g to about 13 g, from about 13 g to about 14 g, from about 14 g to about 15 g, from about 15 g to about 16 g, from about 16 g to about 17 g, from about 17 g to about 18 g, from about 18 g to about 19 g, from about 19 g to about 20 g, from about 20 g to about 21 g, from about 21 g to about 22 g, from about 22 g to about 23 g, from about 23 g to about 24 g, or from about 24 g to about 25 g.

A composition described herein can be present in a unit dose serving in an amount of about 1 g, about 2 g, about 3 g, about 4 g, about 5 g, about 6 g, about 7 g, about 8 g, about 9 g, about 10 g, about 11 g, about 12 g, about 13 g, about 14 g, about 15 g, about 16 g, about 17 g, about 18 g, about 19 g, about 20 g, about 21 g, about 22 g, about 23 g, about 24 g, or about 25 g. In some embodiments, a composition described herein is present in a unit dose serving in an amount of about 10 g, 12 g, 15 g, 20 g, or 24 g.

In some embodiments, a composition described herein is present in a unit dose serving in an amount of about 12 g. In some embodiments, a composition described herein is present in a unit dose serving in a sachet in an amount of about 12 g. In some embodiments, a composition described herein is present in a unit dose serving in an amount of about 15 g. In some embodiments, a composition described herein is present in a unit dose serving in a sachet in an amount of about 15 g. In some embodiments, a composition described herein is present in a unit dose serving in an amount of about 24 g. In some embodiments, a composition described herein is present in a unit dose serving in a sachet in an amount of about 24 g.

In some embodiments, a dose of a composition of the disclosure can be expressed in terms of an amount of the drug divided by the mass of the subject, for example, milligrams of drug per kilograms of subject body mass. In some embodiments, a composition is provided in an amount ranging from about 100 mg/kg to about 150 mg/kg, about 150 mg/kg to about 200 mg/kg, about 200 mg/kg to about 250 mg/kg, about 250 mg/kg to about 300 mg/kg, or about 300 mg/kg to about 350 mg/kg. In some embodiments, a composition is provided in an amount of about 100 mg/kg, about 150 mg/kg, about 200 mg/kg, about 250 mg/kg, about 300 mg/kg, or about 350 mg/kg.

A composition described herein can be provided to a subject to achieve an amount of protein per body weight of the subject. In some embodiments, a composition described herein can be provided to a subject to achieve a range from about 0.2 g protein/kg to about 0.4 g protein/kg, about 0.4 g protein/kg to about 0.6 g protein/kg, about 0.6 g protein/kg to about 0.8 g protein/kg, or about 0.8 g protein/kg to about 1 g protein/kg of body weight of the subject. In some embodiments, a composition described herein can be provided to a subject to achieve a range from about 0.6 g protein/kg to about 0.8 g protein/kg of body weight of the subject.

A composition described herein can be provided to a subject in one or more servings per day. In some embodiments, 1 serving, 2 servings, 3 servings, 4 servings, 5 servings, 6 servings, 7 servings, 8 servings, 9 servings, 10 servings, 11 servings, or 12 servings of a composition described herein is provided to a subject in one day. In some embodiments, 3 servings of a composition described herein is provided to a subject in one day. In some embodiments, 6 servings of a composition described herein is provided to a subject in one day. In some embodiments, 9 servings of a composition described herein is provided to a subject in one day.

### Methods of Administration

A composition of the disclosure can be administered to a subject, and the administration can be accompanied by a food-based diet low in or substantially devoid of at least one amino acid. In some embodiments, administration of a composition of the disclosure is accompanied by a food-based diet low in or substantially devoid of one amino acid. In some embodiments, administration of a composition of the disclosure is accompanied by a food-based diet low in or substantially devoid of serine. In some embodiments, administration of a composition of the disclosure is accompanied by a food-based diet low in or substantially devoid of glycine. In some embodiments, administration of a composition of the disclosure is accompanied by a food-based diet low in or substantially devoid of two amino acids or salts thereof. In some embodiments, administration of a composition of the disclosure is accompanied by a food-based diet low in or substantially devoid of serine and glycine. In some embodiments, administration of a composition of the disclosure is accompanied by a food-based diet low in or substantially devoid of three amino acids or salts thereof. In some embodiments, administration of a composition of the disclosure is accompanied by a food-based diet low in or substantially devoid of serine, glycine, and proline. In some embodiments, administration of a composition of the disclosure is accompanied by a food-based diet low in or substantially devoid of serine, glycine, and cysteine. In some embodiments, administration of a composition of the disclosure is accompanied by a food-based diet low in or substantially devoid of four amino acids or salts thereof. A salt of an amino acid disclosed herein can be a pharmaceutically acceptable salt.

A composition of the disclosure can be administered to a subject that is on a diet. In some embodiments, a composition of the disclosure is administered to the subject, and the subject is on a diet that is low in protein. In some embodiments, a composition of the disclosure is administered to the subject, and the subject is on a low carbohydrate diet. In some embodiments, a composition of the disclosure is administered to the subject, and the subject is on a high-fat, and low-carbohydrate (e.g. ketogenic type diet). In some embodiments, a composition of the disclosure is administered to the subject, and the subject is on a vegetarian diet. In some embodiments, a composition of the disclosure is administered to the subject, and the subject is on a vegan diet.

In some embodiments, a composition of the disclosure is administered to a subject that is on a low protein diet designed to be low in at least one non-essential amino acid. In some embodiments, a composition of the disclosure is administered to a subject that is on a low protein diet designed to be low in serine and glycine. In some embodiments, a composition of the disclosure is administered to a subject that is on a low protein diet with less than about 2 g/day, about 1.75 g/day, about 1.5 g/day, about 1.25 g/day, about 1 g/day, about 0.75 g/day, or about 0.5 g/day. In some embodiments, a composition of the disclosure is administered to a subject that is on a low protein diet with less than about 500 mg/day, about 450 mg/day, about 400 mg/day, about 350 mg/day, about 300 mg/day, about 250 mg/day, about 200 mg/day, about 150 mg/day, about 100 mg/day, or about 50 mg/day.

Multiple therapeutic agents can be administered in any order or simultaneously. In some embodiments, a composition of the invention is administered in combination with, before, or after treatment with another therapeutic agent. If simultaneously, the multiple therapeutic agents can be provided in a single, unified form, or in multiple forms, for example, as multiple separate pills. The agents can be packed together or separately, in a single package or in a plurality of packages. One or all of the therapeutic agents can be given in multiple doses. If not simultaneous, the timing between the multiple doses can vary to as much as about a month.

Therapeutic agents described herein can be administered before, during, or after the occurrence of a disease or condition, and the timing of administering the composition containing a therapeutic agent can vary. For example, the compositions can be used as a prophylactic and can be administered continuously to subjects with a propensity to conditions or diseases in order to lessen a likelihood of the occurrence of the disease or condition. The compositions can be administered to a subject during or as soon as possible after the onset of the symptoms.

A composition disclosed herein can be administered as soon as is practical after the onset of a disease or condition is detected or suspected, and for a length of time necessary for the treatment of the disease. In some embodiments, the length of time necessary for the treatment of disease is about 12 hours, about 24 hours, about 36 hours, or about 48 hours. In some embodiments, the length of time necessary for the treatment of disease is about 1 day, about 2 days, about 3 days, about 4 days, about 5 days, about 6 days, about 7 days, about 8 days, about 9 days, about 10 days, about 11 days, about 12 days, about 13 days, about 14 days, or about 15 days. In some embodiments, the length of time necessary for the treatment of disease is about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, or about 20 weeks. In some embodiments, the length of time necessary for the treatment of disease is about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, about 19 months, about 20 months, about 21 months, about 22 months, about 23 months, or about 24 months.

In some embodiments, the length of time a compound can be administered can be about 1 day, about 2 days, about 3 days, about 4 days, about 5 days, about 6 days, about 1 week, about 2 weeks, about 3 weeks, about 4 weeks, about 1 month, about 5 weeks, about 6 weeks, about 7 weeks, about 8 weeks, about 2 months, about 9 weeks, about 10 weeks, about 11 weeks, about 12 weeks, about 3 months, about 13 weeks, about 14 weeks, about 15 weeks, about 16 weeks, about 4 months, about 17 weeks, about 18 weeks, about 19 weeks, about 20 weeks, about 5 months, about 21 weeks, about 22 weeks, about 23 weeks, about 24 weeks, about 6 months, about 7 months, about 8 months, about 9 months, about 10 months, about 11 months, about 1 year, about 13 months, about 14 months, about 15 months, about 16 months, about 17 months, about 18 months, about 19 months, about 20 months, about 21 months, about 22 months about 23 months, about 2 years, about 2.5 years, about 3 years, about 3.5 years, about 4 years, about 4.5 years, about 5 years, about 6 years, about 7 years, about 8 years, about 9 years, or about 10 years. The length of treatment can vary for each subject.

A composition described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Aqueous suspension compositions can be packaged in single-dose non-reclosable containers. Multiple-dose reclosable containers can be used, for example, in combination with or without a preservative.

In some embodiments, a composition is administered to a subject throughout a day. In some embodiments, a composition is administered to a subject with a meal. In some embodiments, a composition is administered to a subject with a snack. In some embodiments, a composition is administered to a subject without a meal. In some embodiments, a composition is administered to a subject through the day in equal intervals. In some embodiments, a first serving is administered before breakfast, a second serving is administered with breakfast, a third serving is administered with lunch, a fourth and fifth serving is administered with dinner, and a sixth serving is administered before bed.

A composition provided herein can be administered in conjunction with other therapies, for example, chemotherapy, radiation, surgery, anti-inflammatory agents, immunotherapy, biologicals, and selected vitamins. The other agents can be administered prior to, after, or concomitantly with the pharmaceutical compositions.

### Methods of Use of a Composition Disclosed Herein.

The present disclosure provides methods for treating a subject. A composition disclosed herein can be used in the treatment of any disease. In some embodiments, a composition disclosed herein is used to treat cancer in a subject in need thereof. Altering the diet and nutrient of a subject can have desired health benefits and can be efficacious in the treatment of disease.

Based on the particular disease and/or need of the patient, the present disclosure provides methods for generalized-treatment recommendation for a subject as well as methods for subject-specific treatment recommendation. Methods for treatments can comprise one of the following steps: determining a level of a nutrient in a subject; detecting a presence or absence of a disease in the subject based upon the determining, and recommending to the subject at least one generalized or subject-specific treatment to ameliorate disease symptoms.

In some embodiments, a composition disclosed herein can be used to manage a disease or condition by a dietary intervention. In some embodiments, a composition disclosed herein can be used as part of a treatment plan for a particular disease or condition.

In some embodiments, the subject has cancer. Cancer is caused by uncontrollable growth of neoplastic cells, leading to invasion of adjacent and distant tissues resulting in death. Cancer cells often have underlying genetic or epigenetic abnormalities that affect both coding and regulatory regions of the genome. Genetic abnormalities in cancer cells can change protein structures, dynamic and expression levels, which in turn alter the cellular metabolism of the cancer cells. Changes in cell cycles can make cancer cells proliferate at a much higher speed than normal cells. With the increased metabolic rate and proliferation, cancer tissues have much higher nutrient demands compared to normal tissues.

Cancer cells have nutrient auxotrophy and have a much higher nutrient demand compared to normal cells. As an adaptation to fulfill the increased nutritional demand, cancer cells can upregulate the glucose and amino acid transporters on the cell membrane to obtain more nutrients from circulation. Cancer cells can also rewire metabolic pathways by enhancing glycolysis and glutaminolysis to sustain a higher rate of ATP production or energy supply. Glucose and amino acids are highly demanded nutrients in cancer cells. Some cancer cell types and tumor tissues are known to be auxotrophic to specific amino acids. Cancers' auxotrophy to different amino acids can render the cancer types vulnerable to amino acid starvation treatments.

When mammalian cells experience amino acid starvation, the cells undergo a homeostatic response to amino acid shortage. Amino acid deficiency can trigger a general amino acid control pathway that involves shifting resources and energy of cells to expression of membrane transporters, growth hormones, and metabolic enzymes for amino acid homeostasis. Up-regulation of membrane transporters can enhance amino acid uptake, and up-regulation of metabolic enzymes can enhance amino acid synthesis. The cells can also recycle proteins and organelles to regenerate non-essential amino acids by autophagy. By general amino acid control pathway and autophagy, cells attempt to maintain amino acid homeostasis. Tumor tissues can also overcome amino acid starvation by enhancing angiogenesis to obtain more nutrient supply.

When homeostasis cannot be achieved upon severe amino acid starvation, cancer cells can inhibit protein synthesis, suppress growth, or undergo programmed cell death. The cell death mechanisms of amino acid starvation can be caspase-dependent apoptosis, autophagic cell death, or ferroptotic cell death. Amino acid transporters, metabolic enzymes, autophagy-associated proteins, and amino acid starvation can be used to control cancer growth.

A method disclosed herein can monitor nutrient consumption by a subject. The nutrient consumption can be measured by taking a biological sample from a subject. The biological sample can be for example, whole blood, serum, plasma, mucosa, saliva, cheek swab, urine, stool, cells, tissue, bodily fluid, sweat, breath, lymph fluid, CNS fluid, and lesion exudates. A combination of biological samples can be used with the methods of the disclosure.

A method of composition of the disclosure can slow the proliferation of cancer cell lines, or kill cancer cells. Non-limiting examples of cancer that can be treated by a compound of the invention include: acute lymphoblastic leukemia, acute myeloid leukemia, adrenocortical carcinoma, AIDS-related cancers, AIDS-related lymphoma, anal cancer, appendix cancer, astrocytomas, basal cell carcinoma, bile duct cancer, bladder cancer, bone cancers, brain tumors, such as cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas, Burkitt lymphoma, carcinoma of unknown primary origin, central nervous system lymphoma, cerebellar astrocytoma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma, germ cell tumors, gallbladder cancer, gastric cancer, gastrointestinal carcinoid tumor, gastrointestinal stromal tumor, gliomas, hairy cell leukemia, head and neck cancer, heart cancer, hepatocellular (liver) cancer, Hodgkin lymphoma, Hypopharyngeal cancer, intraocular melanoma, islet cell carcinoma, Kaposi sarcoma, kidney cancer, laryngeal cancer, lip and oral cavity cancer, liposarcoma, liver cancer, lung cancers, such as non-small cell and small cell lung cancer, lymphomas, leukemias, macroglobulinemia, malignant fibrous histiocytoma of bone/osteosarcoma, medulloblastoma, melanomas, mesothelioma, metastatic squamous neck cancer with occult primary, mouth cancer, multiple endocrine neoplasia syndrome, myelodysplastic syndromes, myeloid leukemia, nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer, ovarian germ cell tumor, pancreatic cancer, pancreatic cancer islet cell, paranasal sinus and nasal cavity cancer, parathyroid cancer, penile cancer, pharyngeal cancer, pheochromocytoma, pineal astrocytoma, pineal germinoma, pituitary adenoma, pleuropulmonary blastoma, plasma cell neoplasia, primary central nervous system lymphoma, prostate cancer, rectal cancer, renal cell carcinoma, renal pelvis and ureter transitional cell cancer, retinoblastoma, rhabdomyosarcoma, salivary gland cancer, sarcomas, skin cancers, skin carcinoma merkel cell, small intestine cancer, soft tissue sarcoma, squamous cell carcinoma, stomach cancer, T-cell lymphoma, throat cancer, thymoma, thymic carcinoma, thyroid cancer, trophoblastic tumor (gestational), cancers of unknown primary site, urethral cancer, uterine sarcoma, vaginal cancer, vulvar cancer, Waldenström macroglobulinemia, and Wilms tumor.

### Kits

Compositions of the invention can be packaged as a kit. In some embodiments, a kit includes written instructions on the administration/use of the composition. The written material can be, for example, a label. The written material can suggest conditions methods of administration. The instructions provide the subject and the supervising physician with the best guidance for achieving the optimal clinical outcome from the administration of the therapy. The written material can be a label. In some embodiments, the label can be approved by a regulatory agency, for example the U.S. Food and Drug Administration (FDA), the European Medicines Agency (EMA), or other regulatory agencies.

### Radiation Therapy

Radiation therapy, or radiotherapy, is a therapy using ionizing radiation as a part of cancer treatment to control or kill malignant cells and is normally delivered by a linear accelerator. Ionizing radiation damages the DNA of cancerous tissue, resulting in cellular death. Radiation therapy can be curative in a number of types of cancer if localized to one area of the body. In some embodiments, the methods and compositions of the disclosure can be administered in combination with a second therapy, for example, radiotherapy. In some embodiments, radiotherapy can be used with a method or composition of the disclosure because radiotherapy can control cell growth.

In some embodiments, radiotherapy can be used in combination with a method or composition of the disclosure to prevent or reduce the likelihood of tumor recurrence after surgery to remove a primary malignant tumor. In some embodiments, radiotherapy and chemotherapy can be used in combination with a method or composition of the disclosure. In some embodiments, the methods and compositions of the disclosure can be administered in combination with radiotherapy to treat a cancer. In some embodiments, the methods and compositions of the disclosure can be administered in combination with radiotherapy to reduce symptoms of a cancer. In some embodiments, the methods and compositions of the disclosure can be administered in combination with radiotherapy to slow the growth of a cancer.

In some embodiments, the radiotherapy is external beam radiation therapy. External beam radiation therapy uses a machine that locally aims radiation at a cancer. In some embodiments, the radiotherapy is internal beam radiation therapy. In some embodiments, external beam radiation can be used to shrink tumors to treat pain, trouble breathing, or loss of bowel or bladder control. In some embodiments, the external-beam radiation therapy is three-dimensional conformal radiation therapy (3D-CRT). In some embodiments, the external-beam radiation therapy is intensity modulated radiation therapy (IMRT). In some embodiments, the external-beam radiation therapy is proton beam therapy. In some embodiments, the external-beam radiation therapy is image-guided radiation therapy (IGRT). In some embodiments, the external-beam radiation therapy is stereotactic radiation therapy (SRT).

Internal radiation therapy is a treatment that places a source of radiation in the subject's body. In some embodiments, the source of radiation is a liquid. In some embodiments, the source of radiation is a solid. In some embodiments, the internal radiotherapy uses a permanent implant. In some embodiments, the internal radiotherapy is a temporary internal radiotherapy, for example, a needle, tube, or applicator. In some embodiments, the solid source of radiation is used in brachytherapy. In some embodiments, seeds, ribbons, or capsules containing a radiation source are placed in a subject's body. In some embodiments, the radiotherapy is brachytherapy, where a radioactive source is placed inside or next to an area requiring treatment. In some embodiments, the radiotherapy is total body irradiation (TBI) in preparation for a bone marrow transplant.

In some embodiments, the radiotherapy is intraoperative radiation therapy (IORT). In some embodiments, the radiotherapy is systemic radiation therapy. In some embodiments, the radiotherapy is radioimmunotherapy. In some embodiments, the radiotherapy uses a radiosensitizer or a radioprotector.

In some embodiments, brachytherapy is used to treat a cancer of the head, neck, breast, cervix, prostate, or eye. In some embodiments, a systemic radiation therapy such as radioactive iodine, or I-131, can be used to treat thyroid cancer. In some embodiments, targeted radionuclide therapy can be used to treat advanced prostate cancer or a gastroenteropancreatic neuroendocrine tumor (GEP-NET).

In some embodiments, shaped radiation beams can be aimed from several angles of exposure to intersect at the tumor while sparing normal tissue. In some embodiments, a tumor absorbs a much larger dose of radiation than does a surrounding healthy tissue.

In some embodiments, a subject or tumor can be treated with about 0.5 Gray (Gy), about 1 Gy, about 1.5 Gy, about 2 Gy, about 2.5 Gy, about 3 Gy, about 3.5 Gy, about 4 Gy, about 4.5 Gy, about 5 Gy, about 5.5 Gy, about 6 Gy, about 6.5 Gy, about 7 Gy, about 7.5 Gy, about 8 Gy, about 8.5 Gy, about 9 Gy, about 9.5 Gy, or about 10 Gy. In some embodiments, a subject or tumor can be treated with about 5 Gy, about 10 Gy, about 15 Gy, about 20 Gy, about 25 Gy, about 30 Gy, about 35 Gy, about 40 Gy, about 45 Gy, about 50 Gy, about 55 Gy, about 60 Gy, about 65 Gy, about 70 Gy, about 75 Gy, about 80 Gy, about 85 Gy, about 90 Gy, about 95 Gy, or about 100 Gy of radiation therapy. In some embodiments, a subject or tumor can be treated with about 5 Gy of radiation therapy. In some embodiments, a subject or tumor can be treated with about 10 Gy of radiation therapy. In some embodiments, a subject or tumor can be treated with about 20 Gy of radiation therapy.

In some embodiments a subject or tumor can be treated with from about 5 Gy to about 10 Gy; about 10 Gy to about 15 Gy; about 15 Gy to about 20 Gy; about 20 Gy to about 25 Gy; about 25 Gy to about 30 Gy; about 30 Gy to about 35 Gy; about 35 Gy to about 40 Gy; about 40 Gy to about 45 Gy; about 45 Gy to about 50 Gy; about 50 Gy to about 55 Gy; about 55 Gy to about 60 Gy; about 60 Gy to about 65 Gy; about 65 Gy to about 70 Gy; about 70 Gy to about 75 Gy; or about 75 Gy to about 80 Gy. In some embodiments a subject or tumor can be treated with from about 5 Gy to about 10 Gy. In some embodiments a subject or tumor can be treated with from about 20 Gy to about 40 Gy. In some embodiments a subject or tumor can be treated with from about 40 Gy to about 60 Gy.

In some embodiments, one cycle of radiation therapy can comprise the subject or tumor being treated with radiation over a number of days. In some embodiments, the radiation can be occur over 1 day, 2 days, 3 days, 4 days, 5 days, 6 days, 7 days, 8 days, 9 days, 10 days, 11 days, 12 days, 13 days, or 14 days. In some embodiments, one cycle of radiation therapy can comprise the subject or tumor being treated with radiation over 4 days. In some embodiments, one cycle of radiation therapy can comprise the subject or tumor being treated with radiation over 5 days.

In some embodiments, one cycle of radiation can comprise administering 10 Gy over 5 days, for example, 2 Gy a day for 5 days. In some embodiments, one cycle of radiation can comprise administering 15 Gy over 5 days, for example, 3 Gy a day for 5 days. In some embodiments, one cycle of radiation can comprise administering 20 Gy over 5 days, for example, 4 Gy a day for 5 days. In some embodiments, one cycle of radiation can comprise administering 25 Gy over 5 days, for example, 5 Gy a day for 5 days.

In some embodiments, one cycle of radiation therapy can be repeated over a period of time. In some embodiments, a cycle of radiation therapy can be repeated for 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, or 16 weeks.

In some embodiments, a composition of the disclosure can be administered simultaneously with administration of a radiotherapy. In some embodiments, a composition of the disclosure can be administered simultaneously with a radiotherapy for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 days. In some embodiments, a composition of the disclosure can be administered simultaneously with administration of a radiotherapy for 5 days. In some embodiments, a composition of the disclosure can be administered simultaneously with administration of a radiotherapy for 7 days.

In some embodiments, the composition of the disclosure is administered 1 day, 2 days, 3 days, 4 days, 5 days, 6 days, 7 days, 8 days, 9 days, 10 days, 11 days, 12 days, 13 days, or 14 days before a subject is treated with radiotherapy. In some embodiments, the composition of the disclosure is administered 1 day before a subject is treated with radiotherapy. In some embodiments, the composition of the disclosure is administered 2 days before a subject is treated with radiotherapy. In some embodiments, the composition of the disclosure is administered 3 days before a subject is treated with radiotherapy. In some embodiments, the composition of the disclosure is administered 4 days before a subject is treated with radiotherapy.

In some embodiments, a subject can be treated with a composition of the disclosure and radiotherapy, then go off treatment before beginning a subsequent treatment cycle with the composition and radiotherapy. In some embodiments, the length of the treatment period and off-treatment period are identical. In some embodiments, the length of the treatment period and off-treatment period are different. In some embodiments, the length of the treatment period is longer than the off-treatment period. In some embodiments, the length of the treatment period is shorter than the off-treatment period.

In some embodiments, the length of a treatment period with a composition and radio therapy is 1 day, 2 days, 3 days, 4 days, 5 days, 6 days, 7 days, 8 days, 9 days, 10 days, 11 days, 12 days, 13 days, or 14 days, and the length of off-treatment period is 1 day, 2 days, 3 days, 4 days, 5 days, 6 days, 7 days, 8 days, 9 days, 10 days, 11 days, 12 days, 13 days, or 14 days. In some embodiments, the length of the treatment period is 5 days, and the length of the off-treatment period is 2 days. In some embodiments, the length of the treatment period is 4 days, and the length of the off-treatment period is 3 days. In some embodiments, the length of the treatment period is 3 days, and the length of the off-treatment period is 4 days. In some embodiments, the length of the treatment period is 2 days, and the length of the off-treatment period is 5 days.

In some embodiments, a cycle of a treatment period and an off-treatment period is repeated for 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, or 16 weeks.

In some embodiments, a composition of the disclosure and radiotherapy are administered with a high fat diet. In some embodiments, the high fat diet is a diet that has greater than about 50%, about 60%, about 70%, about 80%, or about 90% daily calories from fat. In some embodiments, a composition of the disclosure and radiotherapy are administered with a low carbohydrate diet. In some embodiments, the low carbohydrate diet is a diet with less than about 50%, about 40%, about 30%, about 20%, about 10%, or about 5% daily calories from carbohydrates. In some embodiments, a composition of the disclosure and radiotherapy are administered with a low protein diet. In some embodiments, the low protein diet is a diet with less than about 15%, about 14%, about 13%, about 12%, about 11%, about 10%, about 9%, about 8%, about 7%, about 6%, about 5%, about 4%, about 3%, about 2%, or about 1% of daily calories from whole protein. In some embodiments, the low protein diet has a whole protein amount of less than about 50 g/day, about 40 g/day, about 30 g/day, about 20 g/day, or about 10 g/day. In some embodiments, a composition of the disclosure and radiotherapy are administered with a high fat, low carbohydrate, and low protein diet. In some embodiments, a composition of the disclosure is administered with a normal diet.

Combination Therapy with Immunotherapies

In some embodiments, an amino acid starvation therapy of the disclosure can be used in combination with a chemotherapeutic regimen. In some embodiments, the chemotherapeutic regimen is an immunotherapy. In some embodiments, the immunotherapy is an antibody therapy. In some embodiments, the antibody therapy is treatment with alemtuzumab, rituximab, ibritumomab tiuxetan, or ofatumumab. In some embodiments, the immunotherapy is an interferon. In some embodiments, the interferon is interferon α. In some embodiments, the immunotherapy is an interleukin, for example, IL-2. In some embodiments, the immunotherapy is an interleukin inhibitor, for example, an IRAK4 inhibitor.

In some embodiments, the immunotherapy is a cancer vaccine. In some embodiments, the cancer vaccine is a prophylactic vaccine. In some embodiments, the cancer vaccine is a treatment vaccine. In some embodiments, the cancer vaccine is an HPV vaccine, for example, Gardisil™, Cervarix, Oncophage, or Sipuleucel-T. In some embodiments, the immunotherapy is gp100. In some embodiments, the immunotherapy is a dendridic cell-based vaccine, for example, Ad.p53 DC. In some embodiments, the immunotherapy is a toll-like receptor modulator, for example, TLR-7 or TLR-9. In some embodiments, the immunotherapy is a PD-1, PD-L1, PD-L2, or CTL4-A modulator, for example, nivolumab. In some embodiments, the immunotherapy is an IDO inhibitor, for example, indoximod. In some embodiments, the immunotherapy is an anti-PD-1 monoclonal antibody, for example, MK3475 or nivolumab. In some embodiments, the immunotherapy is an anti-PD-L1 monoclonal antibody, for example, MEDI-4736 or RG-7446. In some embodiments, the immunotherapy is an anti-PD-L2 monoclonal antibody. In some embodiments, the immunotherapy is an anti-CTL1-4 antibody, for example, ipilumumab.

Cancer cells can change cellular metabolism to support elevated energetic and anabolic demands of proliferation of cancer cells. Examples of altered metabolism include aerobic glycolysis (i.e., Warburg effect) and high dependency on non-essential amino acids. One-carbon metabolism encompasses a collection of metabolic pathways that allow cells to generate and use molecules containing single carbons. One-carbon units (i.e., methyl groups) are carried and activated for use by tetrahydrofolates (THF), derived from dietary folate. Cells require one-carbon units to support nucleotide synthesis, methylation reactions and reductive metabolism. Cancer cells are dependent on the one-carbon pathways for supporting high proliferative rates, and one-carbon metabolism is crucial for cancer cell proliferation.

THF-dependent one-carbon metabolism is a critical metabolic process underpinning cellular proliferation supplying carbons for the synthesis of nucleotides incorporated into DNA and RNA. Tryptophan is a theoretical source of one-carbon units through metabolism by indoleamine 2,3-dioxygenase 1 (IDO1). In IDO1 expressing cancer cells, tryptophan is a bona fide one-carbon donor for purine nucleotide synthesis both in vitro and in vivo.

In cancer cell metabolism, serine is considered the predominant source of one-carbon units. Serine is obtained either by de novo synthesis from the glycolytic intermediate 3-phosphoglycerate via the serine synthesis pathway (SSP), or by uptake from the extracellular environment. Some cancer cells display increased SSP enzyme expression in order to meet cellular serine demands, whereas others rely predominantly on serine uptake. Serine hydroymethyltransferases (SHMT1 and SHMT2) directly catalyze the conversion of serine into glycine and the release of a one-carbon, which enters the THF cycle.

The amino acids glycine, histidine and tryptophan are also potential one-carbon donors. Glycine can provide one-carbon units through the glycine cleavage system (GCS). Histidine catabolism can also yield one-carbon units and can further sensitize cancer cells to anti-folate treatment due to a decrease in free THF pools.

As an essential amino acid, tryptophan is critical for protein synthesis, but is also a precursor for 5-hydroxytryptamine and kynurenine production. In the kynurenine pathway, the initial and rate-limiting step is the conversion of tryptophan to formyl-kynurenine. Three enzymes are capable of catalyzing this reaction: IDO1 IDO2, and TDO. Both IDO2 and TDO have low expression levels and limited tissue specificity, and IDO1 is considered the predominant form. Formyl-kynurenine spontaneously forms kynurenine, with the release of a molecule of formate. Formate can enter the one-carbon cycle by directly reacting with THF and it is via this pathway that tryptophan can serve as a one-carbon donor.

IDO1 activity depletes tryptophan and increases kynurenine in the tumor microenvironment, causing a range of effects on immune cells. Tryptophan depletion decreases tumor infiltrating T-cell activity, and kynurenine decreases effector T-cell proliferation and supports the differentiation of immunosuppressive T-regulatory cells through binding of the aryl hydrocarbon receptor. The tumor micro-environmental effects provide an immunologically permissive environment for tumor growth. The kynurenine pathway has several metabolic outputs, including: reactive oxygen species (superoxide) levels, one-carbon metabolism, synthesis of NAD(P)+, synthesis of alanine and entry of carbons (via α-ketoadipate) into the TCA cycle.

Disclosed herein is a method of treating a cancer in a subject in need thereof, the method comprising a) administering to the subject a therapeutically-effective amount of a pharmaceutical composition, wherein the pharmaceutical composition is substantially devoid of at least two amino acids; and b) an IDO1 inhibitor. In some embodiments, the at least two amino acids is serine and glycine.

In some embodiments, the IDO1 inhibitor is indoximod (D-1MT; NLG-8189), 4-phenylimidazole (4-PI), N3-benzyl substituted 4-PI, ortho-hydroxy 4-PI, navoximod, or epacadostat. In some embodiments, the IDO1 inhibitor is epacadostat.

In some embodiments, a composition of the disclosure and an IDO1 inhibitor can be used to treat a cancer. In some embodiments, the cancer is pancreatic cancer. In some embodiments, the cancer is colon cancer. In some embodiments, the cancer is breast cancer. In some embodiments, the cancer is cervical cancer. In some embodiments, the cancer is lung cancer.

In some embodiments, the IDO1 inhibitor is administered 1, 2, 3, 4, or 5 times daily in combination with an amino acid starvation therapy. In some embodiments, the IDO1 inhibitor is administered once daily in combination with an amino acid starvation therapy. In some embodiments, the IDO1 inhibitor is administered twice daily in combination with an amino acid starvation therapy. In some embodiments, the IDO1 inhibitor is administered three times daily in combination with an amino acid starvation therapy.

In some embodiments, the IDO1 inhibitor is administered in an amount of from about 10 mg to about 50 mg, from about 50 mg to about 100 mg, from about 100 mg to about 150 mg, from about 150 mg to about 200 mg, from about 200 mg to about 250 mg, from about 250 mg to about 300 mg, from about 300 mg to about 350 mg, from about 350 mg to about 400 mg, from about 400 mg to about 450 mg, or about 450 mg to about 500 mg. In some embodiments, the IDO1 inhibitor is administered in an amount of from about 50 mg to about 100 mg. In some embodiments, the IDO1 inhibitor is administered in an amount of from about 100 mg to about 150 mg. In some embodiments, the IDO1 inhibitor is administered in an amount of from about 250 mg to about 300 mg.

In some embodiments, the IDO1 inhibitor is administered in an amount of about 10 mg, about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg. In some embodiments, the IDO1 inhibitor is administered in an amount of about 25 mg. In some embodiments, the IDO1 inhibitor is administered in an amount of about 50 mg. In some embodiments, the IDO1 inhibitor is administered in an amount of about 100 mg. In some embodiments, the IDO1 inhibitor is administered in an amount of about 300 mg.

In some embodiments, about 25 mg of epacadostat is administered to a subject in combination with serine and glycine starvation therapy. In some embodiments, about 50 mg of epacadostat is administered to a subject in combination with serine and glycine starvation therapy. In some embodiments, about 100 mg of epacadostat is administered to a subject in combination with serine and glycine starvation therapy. In some embodiments, about 300 mg of epacadostat is administered to a subject in combination with serine and glycine starvation therapy.

### EXAMPLES

### Example 1: PHGDH Inhibitor Along with Lack of Serine and Glycine can Impede Growth of Tumor Cell Lines

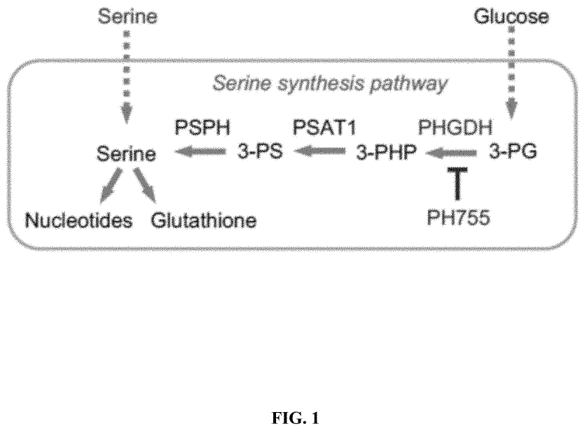

Cells can take up exogenous serine or synthesize serine from the glycolytic intermediate 3-phosphoglycerate (3-PG), using the serine synthesis pathway (FIG. 1). As a non-essential amino acid, serine can be taken up from the environment or newly synthesized through the serine synthesis pathway (SSP). The SSP consists of a three-step enzymatic reaction starting with the NAD+-dependent oxidation of the glycolytic intermediate 3-phosphoglycerate (3-PG) to 3-phosphohydroxypyruvate (3-PHP). This first reaction is catalyzed by phosphoglycerate dehydrogenase (PHGDH), an enzyme that can be targeted by the pharmacological compound PH755. The 3-PHP produced during the PHGDH reaction is then converted into 3-phosphoserine (3-PS) by phosphoserine aminotransferase 1 (PSAT1) in a glutamate-dependent transamination reaction. Finally, phosphoserine phosphatase (PSPH) catalyzes the hydrolysis of 3-PS to produce serine. Serine is involved in numerous metabolic pathways including nucleotide synthesis or glutathione synthesis, a major antioxidant for the cells. Serine availability can thus be targeted by depleting it from the extracellular environment or by inhibition of the SSP using PH755.

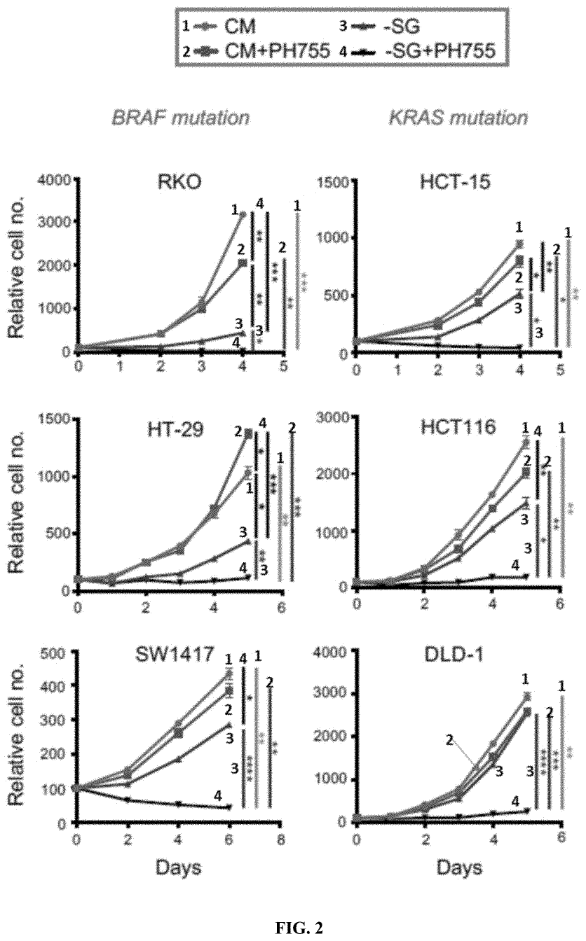

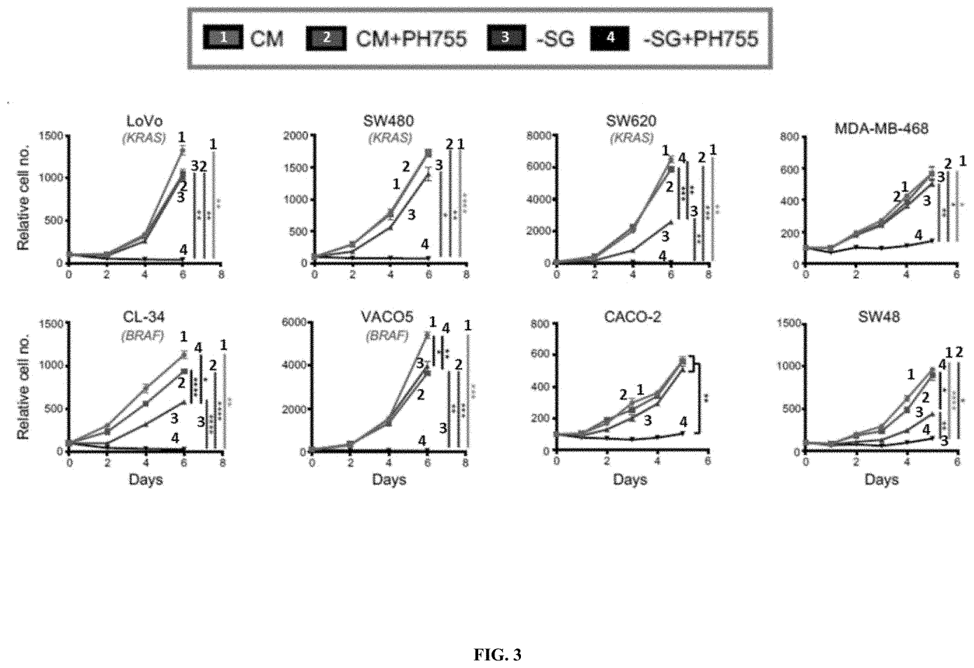

To assess the relative contribution of each of these pathways to the growth of cells in culture, the proliferation of a series of colorectal cancer cell lines grown in complete medium (CM), medium lacking serine and glycine (−SG), CM with PH755 (a PHGDH inhibitor) or a combination of −SG plus PH755 was measured. The response to serine and glycine starvation varied between cell lines ranging from RKO, HT-29, and SW48 cells that showed a significant dependence on exogenous serine and glycine for proliferation, to DLD-1, LoVo, CACO-2 and MDA-MB-468 cells (a breast cancer line previously shown to carry PHGDH amplification) that were not affected by lack of serine and glycine in the medium (FIG. 2 and FIG. 3). There was a trend for colorectal cancer cell lines carrying KRAS mutation (HCT-15, HCT116, DLD-1, LoVo, SW480) to be more resistant to serine and glycine withdrawal compared to those cell lines carrying BRAF mutations (RKO, HT-29, SW1417, CL-34), although SW620 (KRAS mutant) and VACO5 (BRAF mutant) were exceptions to this trend (FIG. 2 and FIG. 3). Genetic alterations—such as amplification of PHGDH in MDA-MB-468 cells—also contribute to the dependence of cancer cells on a supply of exogenous serine. Treatment of the cells with PH755 in complete medium had no clear effect on the proliferation rate of the cells, indicating that at this concentration, the inhibitor has no non-specific inhibitory effect on cell growth. However, combining −SG medium with PH755 completely inhibited the growth of all the cell lines tested (FIG. 2 and FIG. 3).

### Example 2: PHGDH Inhibition Combined with Lack of Serine and Glycine Limits DNA Synthesis, Survival & Organoid Growth

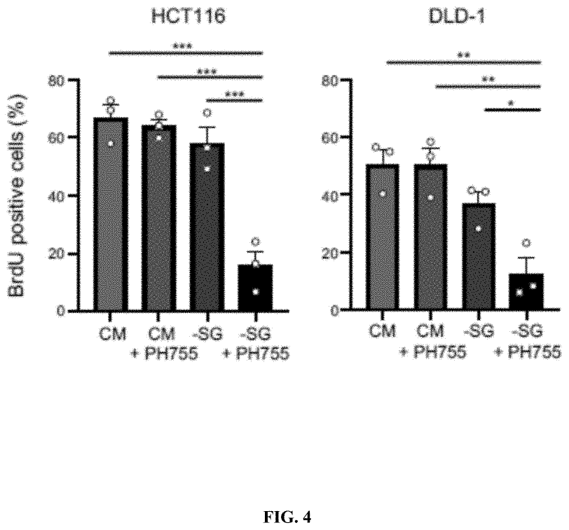

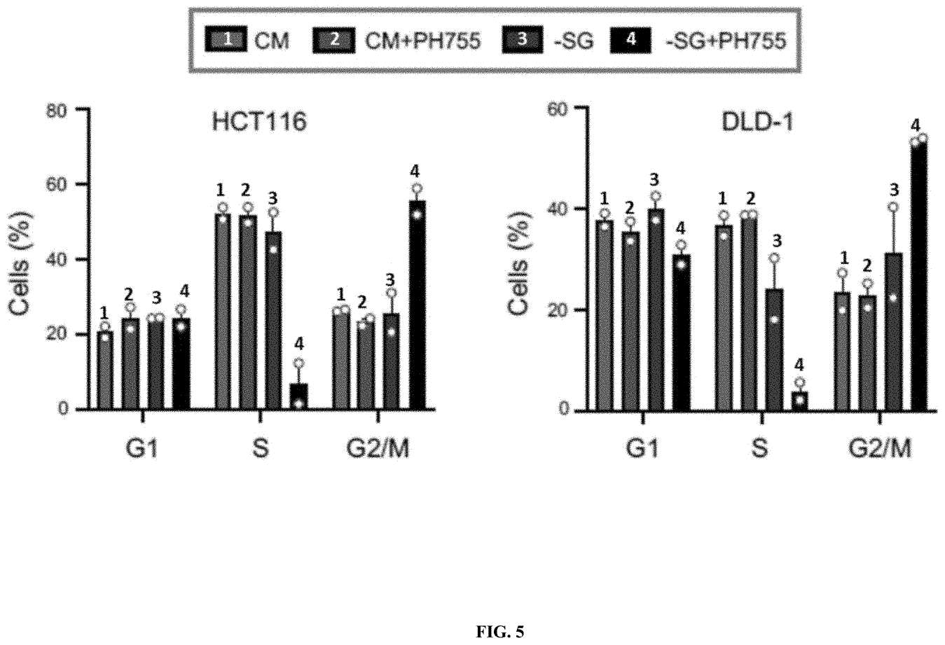

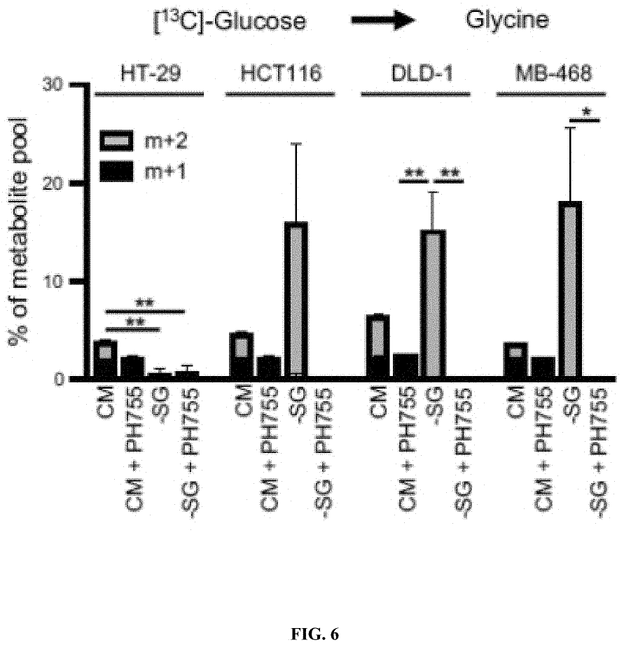

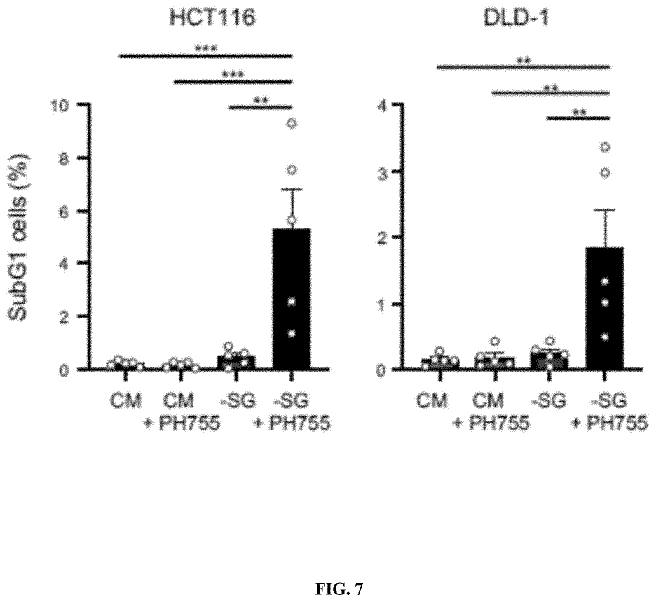

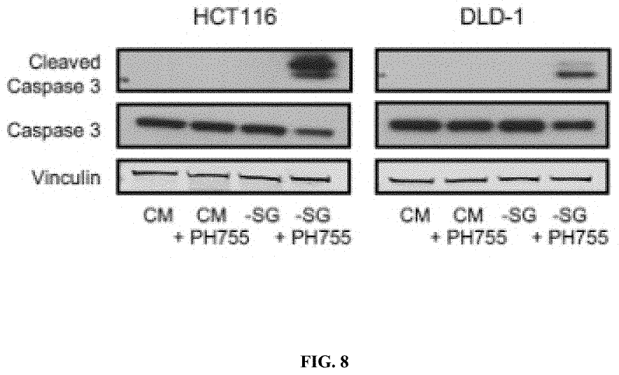

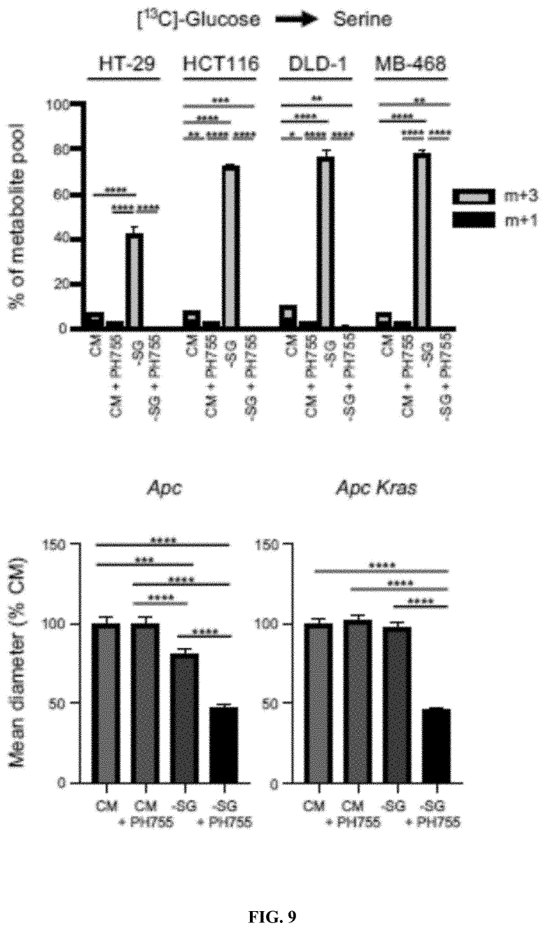

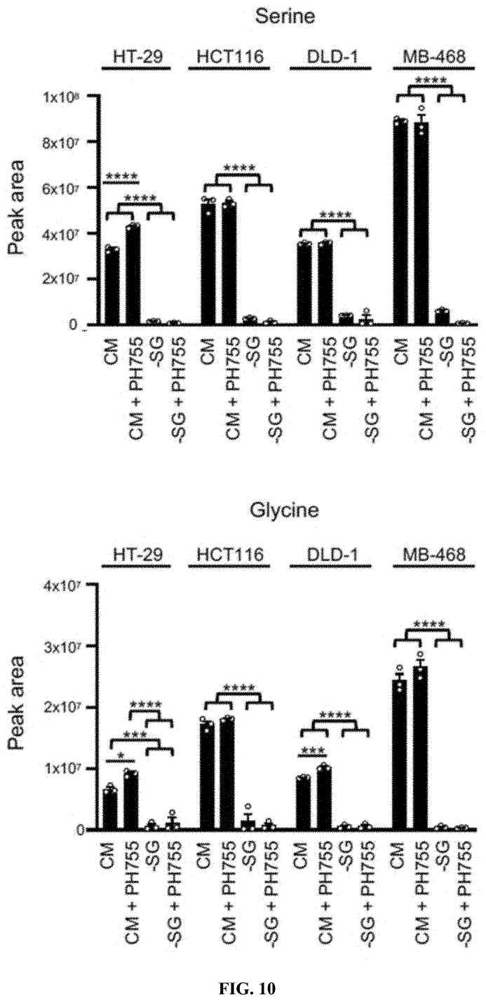

Accompanying this lack of proliferation seen in EXAMPLE 1 was a strong reduction of BrdU incorporation into newly synthesized DNA after 48 hours incubation with −SG medium plus PH755, compared to either treatment alone (FIG. 4 and FIG. 5). The decrease in cells undergoing S-phase was accompanied by an accumulation of cells in G2/M phase (FIG. 5 and FIG. 6) and an increase in the proportion of SubG1 cells in the double-treated condition, indicating an increase in cell death (FIG. 7). The appearance of cleaved-caspase 3 confirmed the induction of apoptosis in cells cultured in −SG medium and treated with PH755 (FIG. 8). Using uniformly labelled glucose, cells grown in the presence of exogenous serine diverted little glucose into serine and glycine synthesis, as reflected by the negligible accumulation of m+3 serine and m+2 glycine (FIG. 9 and FIG. 10), regardless of the presence or absence of PH755. Of note, these cells maintained much higher overall intracellular serine and glycine levels than cells grown in the −SG medium (FIG. 6). When starved of serine and glycine, all the cell lines showed a clear increase in de novo serine synthesis, as indicated by the accumulation of m+3 labelled serine and m+2 labelled glycine (FIG. 9 and FIG. 10). This response was weaker in the HT-29 cells, consistent with their lower ability to proliferate in the absence of exogenous serine. However, treatment of the cells with PH755 completely blocked de novo synthesis of serine and glycine, both in complete medium and under serine and glycine starvation (FIG. 9 and FIG. 10), demonstrating the efficiency of this inhibitor in blocking PHGDH activity and the SSP.

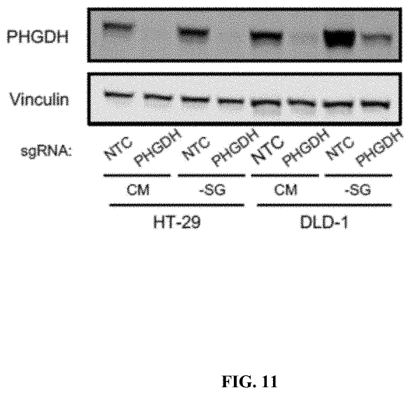

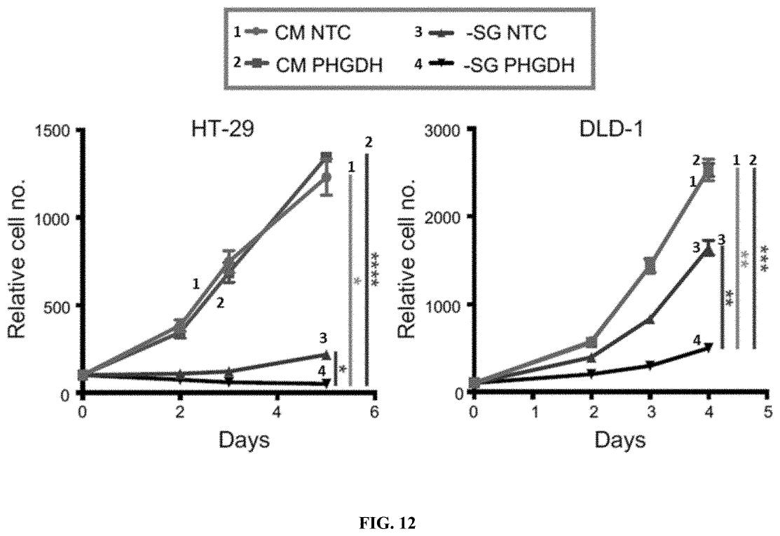

To further verify the specificity of PH755, the effect of genetic deletion of PHGDH was tested. DLD-1 cells showed a strong induction of PHGDH expression in response to serine and glycine starvation, which was much less robust in HT-29 cells—consistent with the relative ability of these cell lines to proliferate in the absence of exogenous serine and glycine (FIG. 11 and FIG. 12). Proliferation of these cells following CRISPR-mediated deletion of PHGDH (FIG. 11 and FIG. 12) mirrored that seen following PH755 treatment (FIG. 2), supporting the function of PH755 as an inhibitor of PHGDH.

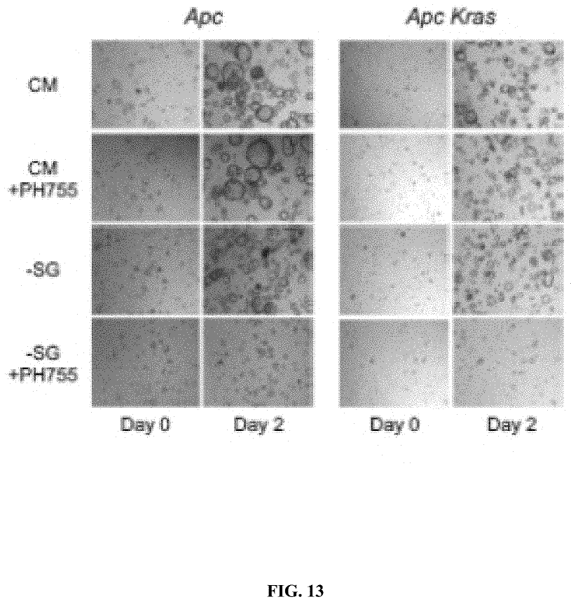

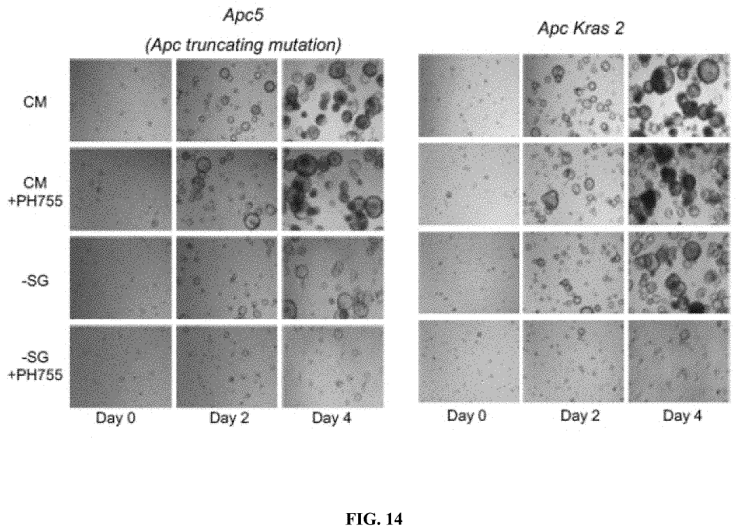

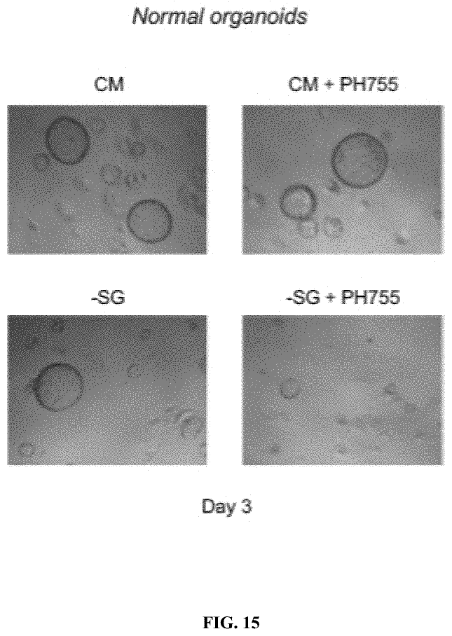

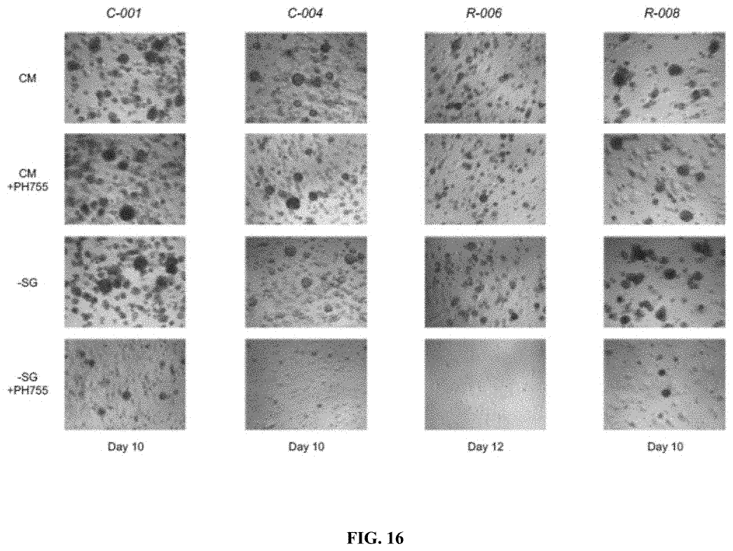

Cells grown in 2D on plastic can show different metabolic requirements compared to cells grown under more physiologically relevant conditions, and the effect of serine and glycine depletion and PH755 treatment on intestinal tumor organoids derived from Vil1-cre^{ER};Apc^{fl/fl} (Apc) or Vil1-cre^{ER};Apc^{fl/fl};Kras^{G12D/+} (Apc Kras) mice (FIG. 13 and FIG. 14) was examined. Organoids derived from Apc mutant tumors showed some sensitivity to serine and glycine depletion, which was not evident in Apc/Kras mutant organoids. Consistent with the observations in 2D cell lines, treatment with PH755 alone did not impact the growth of Apc or Apc/Kras organoids. However, the combination of serine and glycine starvation and PH755 treatment effectively inhibited the growth of both Apc and Apc/Kras organoids (FIG. 13 and FIG. 14). Of note, this effect was not restricted to cancer-derived intestinal organoids, as a substantial reduction in growth was also observed in normal small intestine organoids treated with the combination treatment (FIG. 15). To validate the effect of the double treatment in human cells four patient-derived colorectal cancer organoids with different KRAS status (C-001: WT, C-004: deletion, R-006: Gly12Asp and R-008: Gly13Asp) were tested. While −SG or PH755 treatment alone did not have a marked impact on proliferation, in each case, the combination of drug and inhibitor greatly decreased organoid growth, regardless of KRAS status (FIG. 16).

### Example 3: PHGDH Inhibition Combined with Lack of Serine and Glycine Inhibits Purine and GSH Synthesis

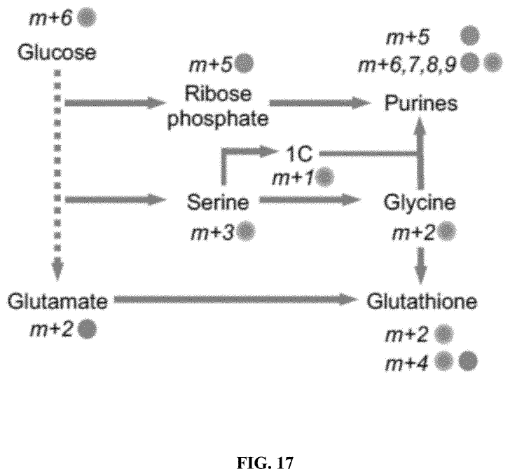

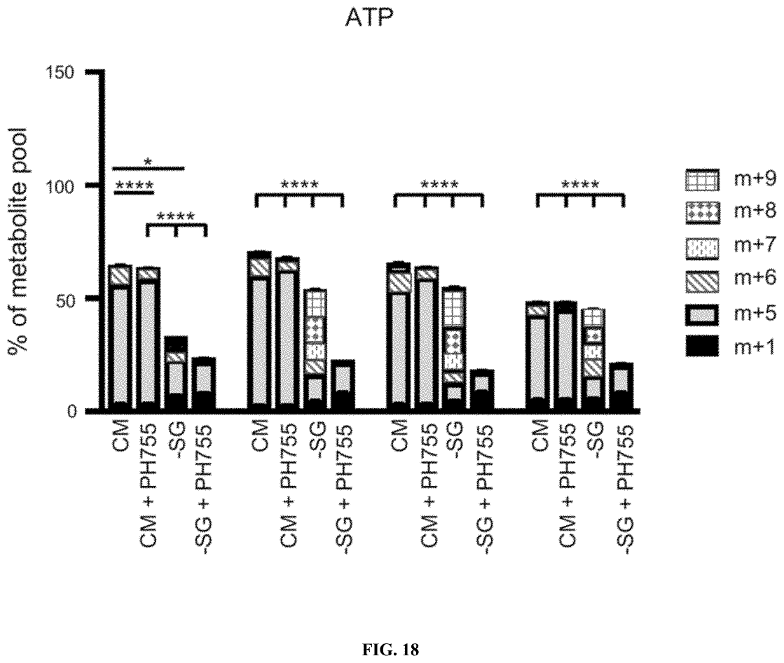

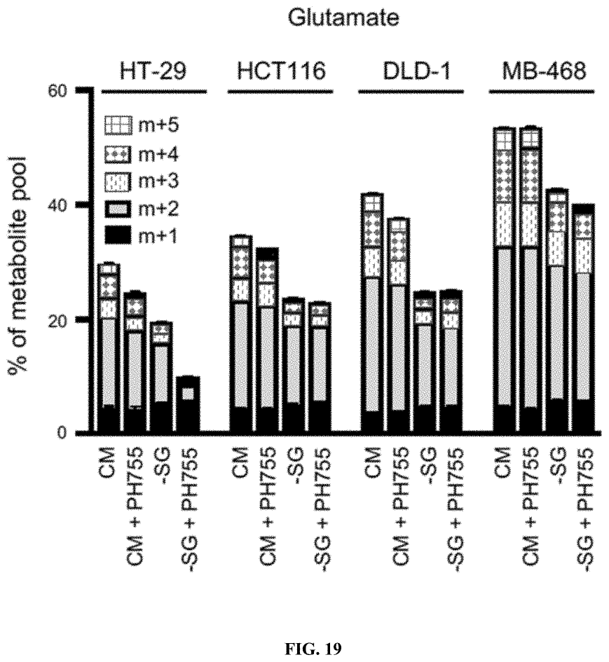

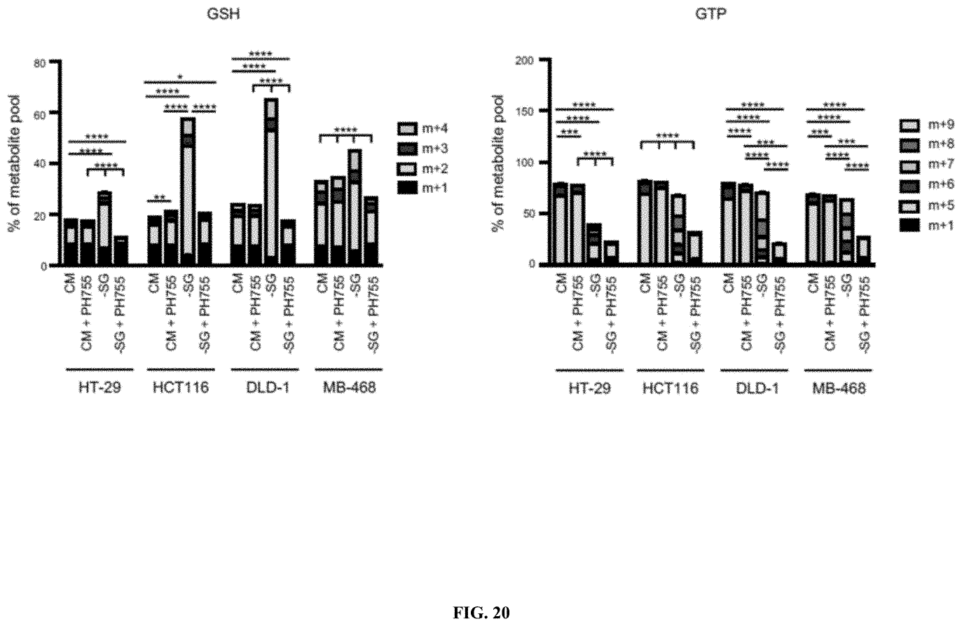

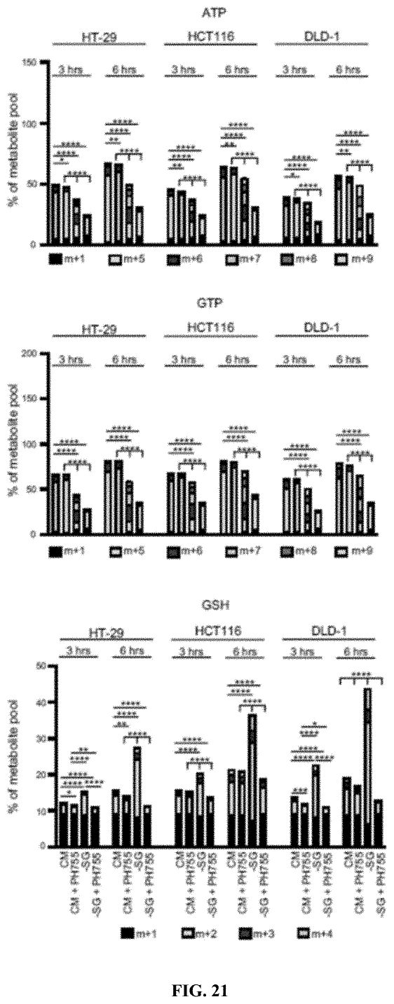

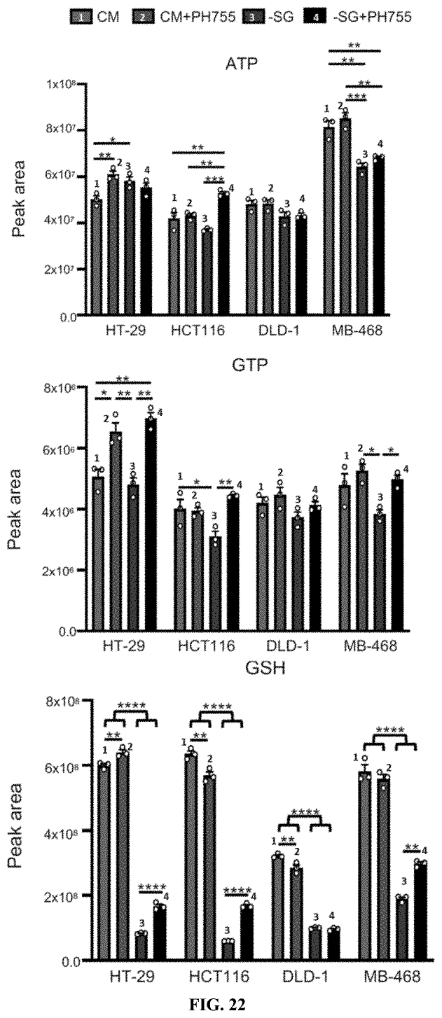

Serine is involved in numerous metabolic pathways, including the provision of one-carbon units and glycine for purine synthesis and the maintenance of redox homeostasis through glutathione production. The contribution of de novo synthesized serine to these pathways can be assessed by following the fate of uniformly carbon-labeled glucose (FIG. 17). Cells grown in serine and glycine showed little evidence of the use of de novo synthesized serine for ATP or GTP synthesis (FIG. 18); rather, the majority of label (m+5) deriving from ribose was synthesized through the pentose phosphate pathway (FIG. 17). Under serine and glycine starvation, cells that were best able to adapt to these conditions (HCT116, DLD1, and MDA-MB-468) accumulated m+6 to m+9 labelled purines, consistent with the incorporation of labelled serine generated through the SSP (FIG. 18). Serine and glycine starvation with PH755 treatment effectively inhibited synthesis of ATP and GTP (FIG. 18). Glutathione can be labelled from glucose derived glycine (m+2) or glutamate (m+2) (FIG. 17), although under these conditions the generation of m+2 glutamate was not impacted by PH755 treatment in most of the cell lines tested (FIG. 19). The increase in the proportion of m+2 and m+4 labelled glutathione detected in response to the removal of exogenous serine and glycine can reflect the increase in SSP activity and production of labelled glycine, a response that was blocked by treatment with PH755 (FIG. 20). Importantly, the inability of the double treated cells to newly synthetize purines and glutathione was evident as early as 3- or 6-hours post-treatment, demonstrating that this response can represent a primary effect of the combination treatment (FIG. 21). Of note, total purine and GSH levels were not decreased in the double treated cells compared to the cells grown in −SG medium, probably reflecting the lack of consumption of these metabolites when proliferation is inhibited (FIG. 22). These results demonstrate that metabolic pathways that are dependent on serine and critical for the growth of cancer cells are efficiently inhibited by a combination of serine and glycine starvation and the PHGDH inhibitor.

### Example 4: Metabolic Rescue of Cells Co-Treated −SG/PHGDHi Treated Cells

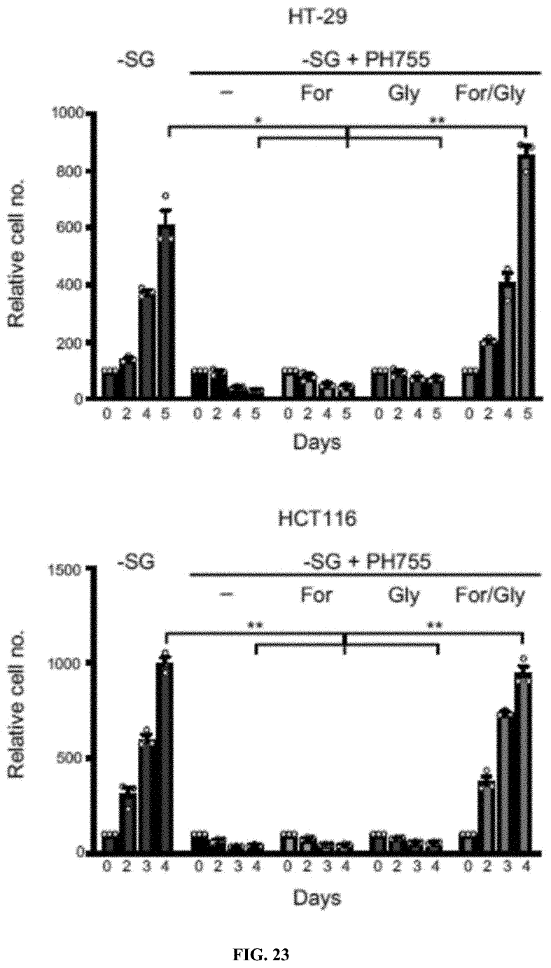

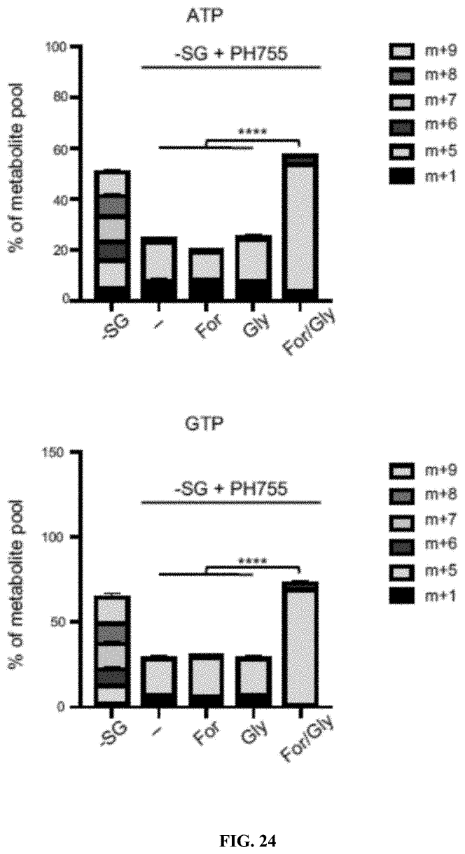

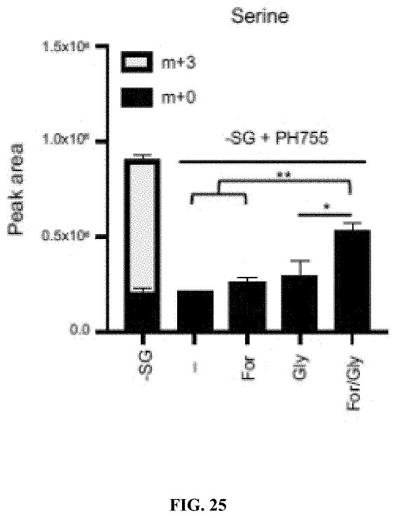

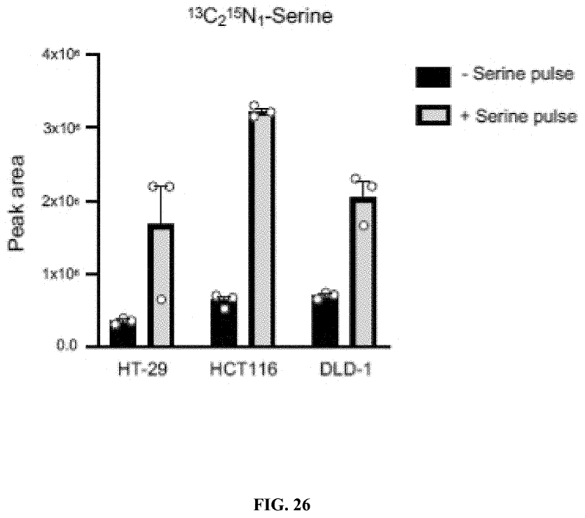

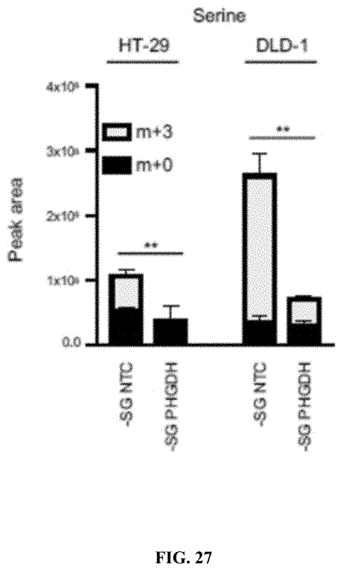

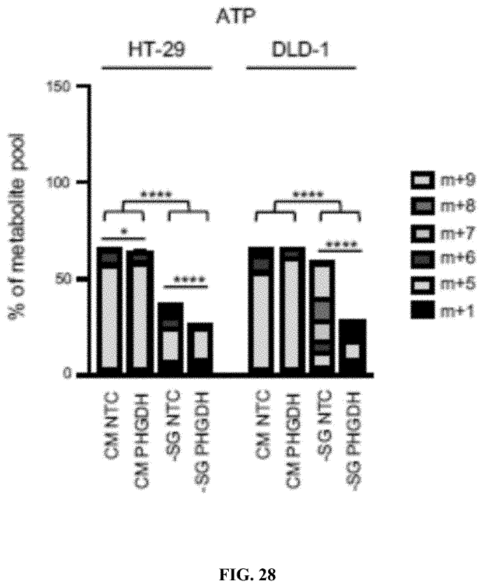

All cells deprived of serine and glycine and treated with PH755 showed a strong growth inhibition (FIG. 2, FIG. 13, FIG. 3, FIG. 17, FIG. 18, and FIG. 20). While supplementation of the double treated cells with either formate (to replenish the one-carbon cycle) or glycine alone did not restore growth, addition of formate and glycine effectively rescued proliferation (FIG. 23). This proliferation rescue was accompanied by the recovery of ATP and GTP synthesis (FIG. 24), and the partial restoration of the pool of unlabeled serine (FIG. 25). Using labelled glycine, it was shown that this pool of serine is generated from glycine and one-carbon units provided by formate, a response that is made more evident following the addition of a pulse of unlabeled serine to allow the labelled serine to accumulate (FIG. 26). These results show that the inhibition of proliferation is a direct effect of inhibition of de novo serine synthesis by PH755, and not a response to any off-target toxicity. The specificity of the metabolic defect induced by PH755 was further supported by the similarity of the response to genetic deletion of PHGDH (FIG. 27, FIG. 28, and FIG. 29).

### Example 5: PHGDHi/−SG Treatment Impairs the General ATF-4 Response

While the effect of PH755 was consistent with a specific inhibition of PHGDH, analysis of the expression of the serine synthesis pathway enzymes in response to PH755 treatment revealed an unexpected response in some of the cell lines. Serine and glycine starvation can lead to the activation of ATF-4, which can mediate a general survival response to metabolic stress. Importantly, serine starvation leads to an ATF-4 dependent induction of expression of the SSP enzymes, so contributing to the ability of the cells to adapt to a reduction in exogenous serine levels.

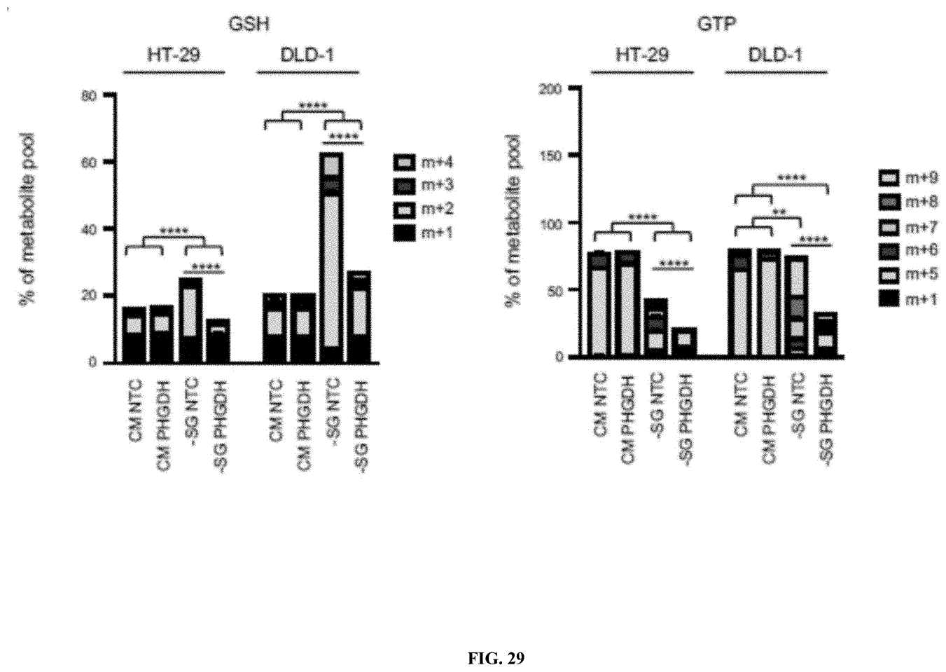

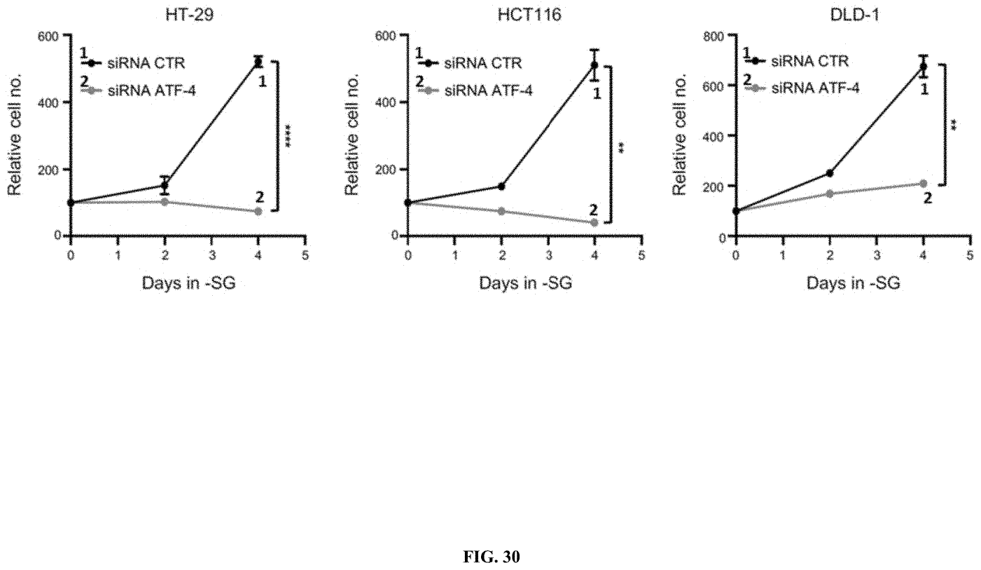

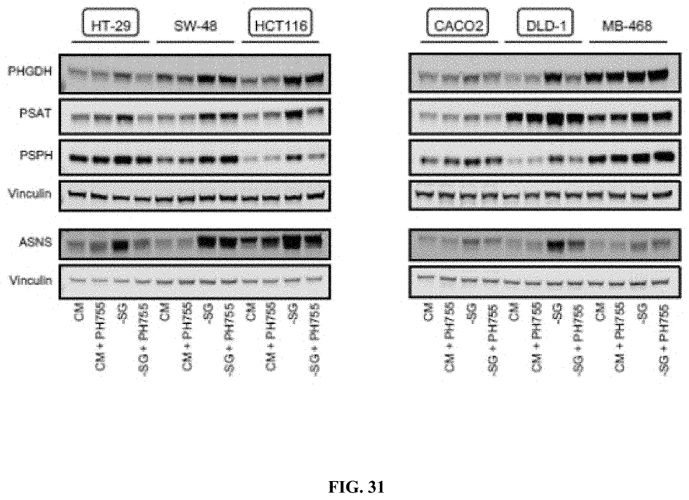

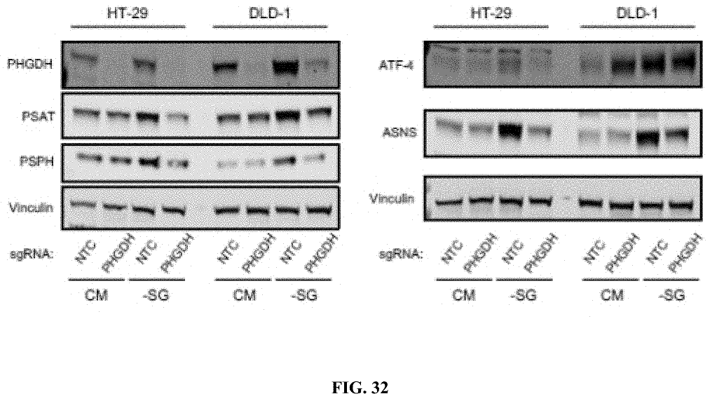

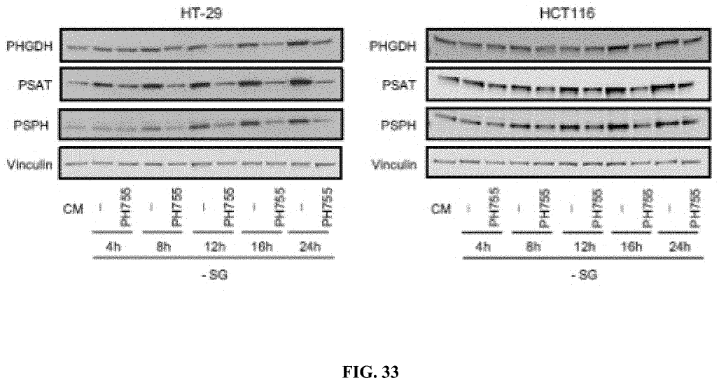

Depletion of ATF-4 resulted in an inability of the cells to adapt and grow under serine and glycine starvation (FIG. 30). As expected, serine and glycine depletion led to an induction of expression of all three SSP enzymes in all the cell lines tested, although this was less robust in MDA-MB-468 cells that constitutively overexpress these enzymes (FIG. 31). However, in four of the colon cancer lines (HT-29, HCT116, CACO2, and DLD-1), further treatment of serine and glycine starved cells with PH755 diminished this increase in SSP enzyme expression (FIG. 31), although this was not seen in SW48 cells. A similar response following serine and glycine starvation and PHGDH deletion confirmed that this was a response to loss of PHGDH activity (FIG. 32). While the decrease in activation of the SSP enzymes is correlated with the growth inhibition seen following serine and glycine starvation and PH755 treatment, the failure of doubly treated cells to induce the SSP enzymes is evident within 4-8 hours of serine and glycine starvation (FIG. 33), suggesting this is a direct response to PHGDH inhibition rather than an indirect response to growth arrest. The loss of ability to induce SSP enzyme expression in response to serine and glycine starvation was accompanied by a general inability to activate an ATF-4 response, as measured by a lack of induction of the canonical ATF-4 target, ASNS (FIG. 31 and FIG. 32). These results suggest that cells can respond to the combination of serine starvation and SSP inhibition differently than to either intervention alone.

### Example 5: PHGDHi/−SG Treatment Inhibits Global Protein Synthesis

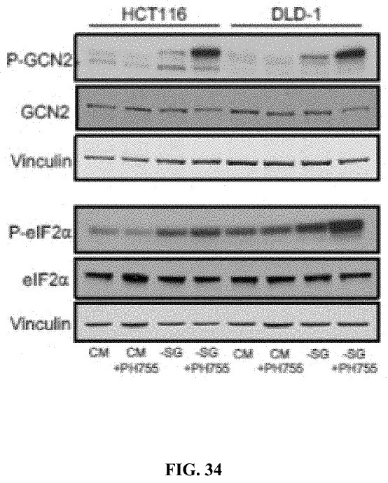

To explore how PHGDH activity affects the ATF-4 response induced following serine and glycine withdrawal, the level of activation of the upstream regulators responsible for ATF-4 induction was examined. In response to amino acid starvation, the accumulation of uncharged tRNA leads to the activation and autophosphorylation of the kinase General Control Nonderepressible 2 (GCN2). GCN2 then phosphorylates the eukaryotic initiation factor 2a (eIF2α) at serine 51, leading to a general downregulation of global translation but selectively inducing the translation of ATF-434. Serine and glycine withdrawal induced the phosphorylation of GCN2 and its target eIF2α in HCT116 and DLD-1 cells (FIG. 34). Interestingly, this induction of GCN2 and eIF2α phosphorylation was sustained or even more pronounced in cells co-treated with PH755 (FIG. 34), demonstrating that the lack of ATF-4 upregulation in the double treated cells was not due to a lack of activation of its upstream regulators. ATF-4 protein levels can also be regulated through ubiquitin dependent proteasomal degradation. However, while treatment of cells with the proteasome inhibitor MG-132 led to a strong accumulation of ATF-4 in cells grown in complete medium, there was no restoration of ATF-4 protein levels or expression of target gene products ASNS and PSAT in cells grown in −SG medium plus PH755 (FIG. 35).

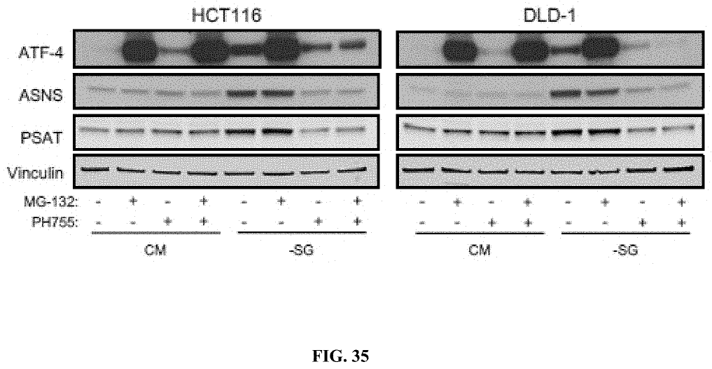

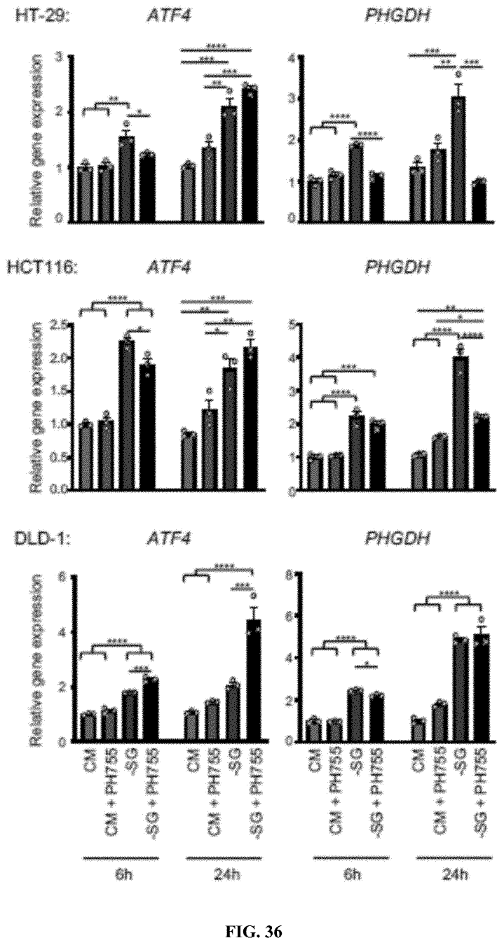

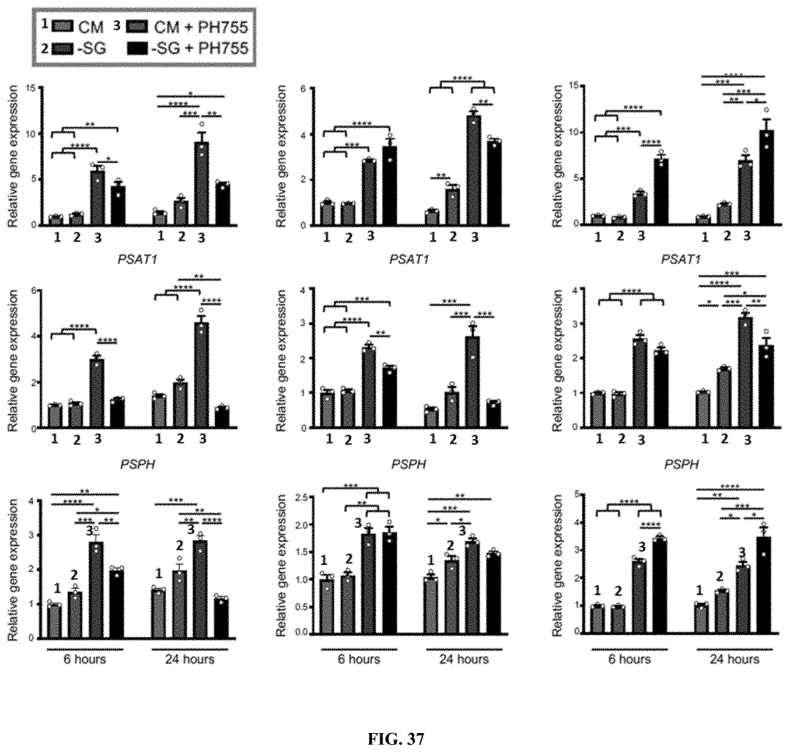

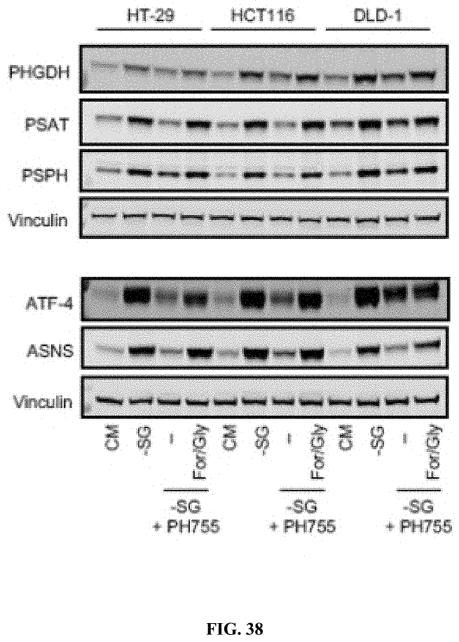

Furthermore, gene expression analysis showed that ATF4 was induced at the transcriptional level by 24 hours after serine and glycine deprivation regardless of the presence of the PHGDH inhibitor (FIG. 36). These data indicate that the lack of ATF-4 induction in double treated cells was not due to increased protein degradation or decreased transcription. As expected, the transcription of ATF-4 target genes, ASNS and SSP enzymes, was strongly up-regulated in serine and glycine starved cells (FIG. 36 and FIG. 37). Interestingly, the transcription of ATF-4 target genes reflected the extent of ATF-4 induction in the different cell lines grown in −SG medium plus PH755. DLD-1 cells, which maintained some induction of ATF-4 under these conditions (FIG. 38), retained the ability to induce expression of PHGDH, ASNS, PSAT and PSPH (FIG. 36 and FIG. 37) while HT-29 and HCT116 cells, which showed a more blunted induction of ATF-4 (FIG. 38) also showed a more severe defect in the ability to transcriptionally activate these ATF-4 target genes (FIG. 36 and FIG. 37).

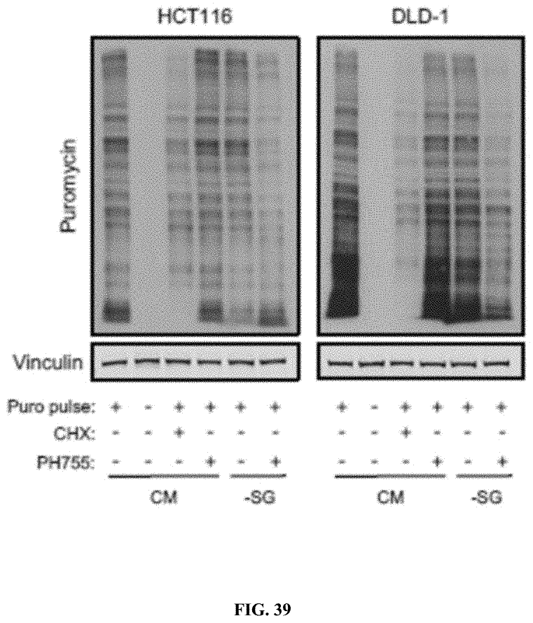

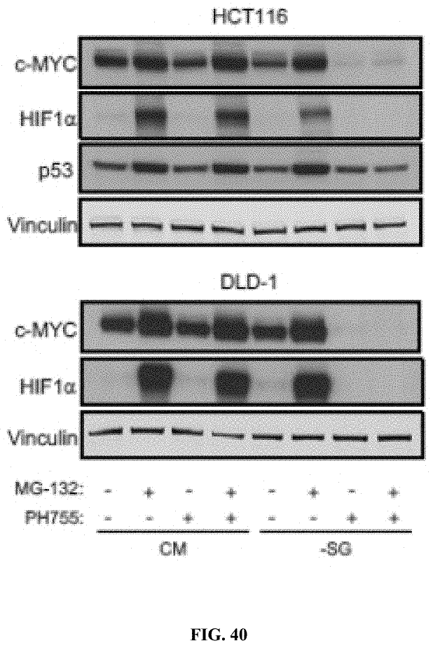

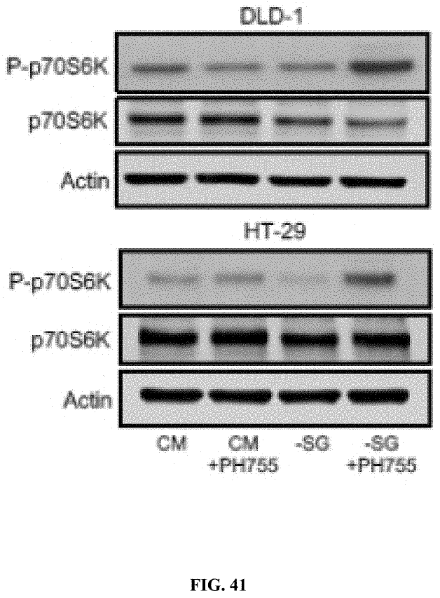

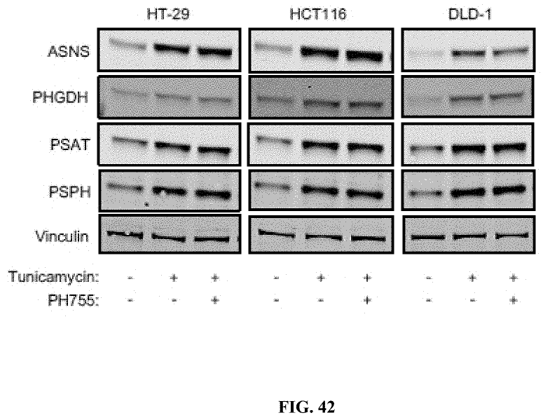

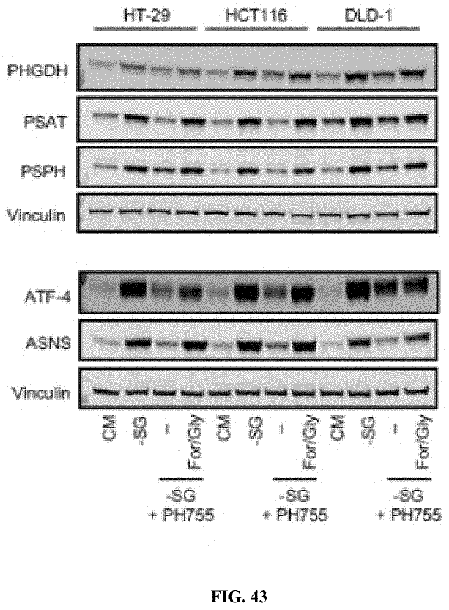

PH755 treatment did not primarily affect the transcription of ATF-4 or ATF-4 target genes but impacted the subsequent expression of each of these proteins. To determine whether this reflected a general inhibition of translation resulting from the dramatic decrease of serine and glycine availability seen in this condition, the incorporation of puromycin, a tyrosyl-tRNA mimetic, into newly synthesized polypeptides in cells grown in CM or −SG medium plus PH755 was analyzed. Interestingly, while a modest decrease in the amount of puromycin-labelled peptides in response to serine/glycine withdrawal was observed, this reduction was much more pronounced in presence of the PHGDH inhibitor (FIG. 39). Consistent with a global inhibition of translation in the double treated cells, the ability of proteasome inhibition to drive the accumulation of short-lived proteins such as c-MYC, HIF1α and p53 was fully blocked in −SG plus PH755 treated cells (FIG. 40). Other conditions that induce a general inhibition of protein synthesis (such as cycloheximide or puromycin treatment), mTORC1 is hyper-activated in the double treated cells, as shown by the accumulation of phosphorylated S6K (FIG. 41). Therefore, the lack of serine and glycine availability triggered by the inhibition of both extracellular and intracellular supplies of these amino acids (FIG. 11) interrupts normal translation and prevents the induction of an ATF-4 mediated protective response. In support of this model, the effect of PHGDH inhibition on the ATF-4 response was specific to serine and glycine deprivation, since treatment with PH755 did not prevent the induction of ATF-4 targets in response to ER stress (FIG. 42). Furthermore, supplementation of the double treated cells with formate and glycine—a treatment that restored some level of serine availability (FIG. 25)—fully rescued the ATF-4 response (FIG. 43). Therefore, in the absence of extracellular serine, PHGDH activity becomes essential to maintain global protein synthesis, allowing the induction of a protective ATF-4 response.

### Example 6: Combining a Serine/Glycine-Free Diet and a PHGDH Inhibitor is Well Tolerated In Vivo

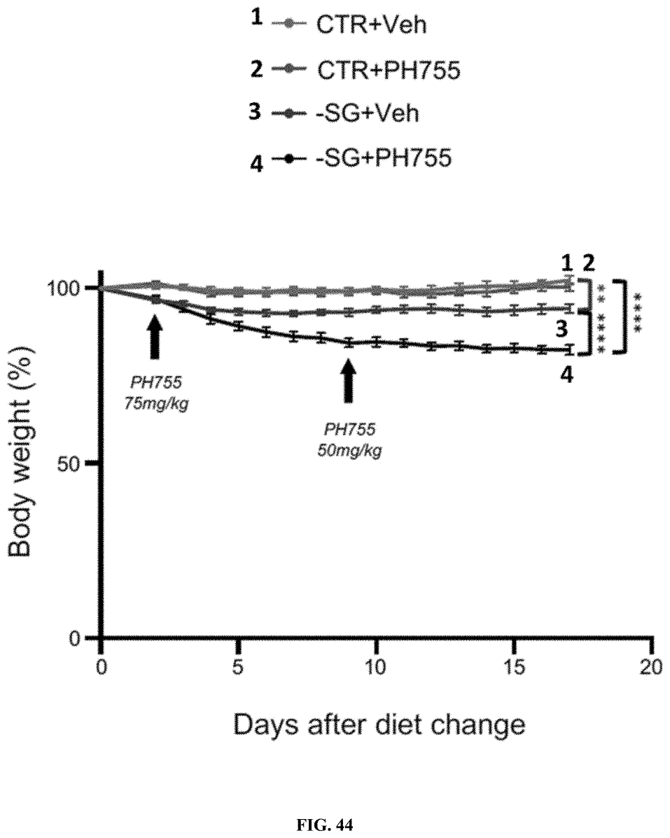

The in vitro data indicate that the growth inhibitory response to serine and glycine depletion is greatly augmented by treatment of cells with the PHGDH inhibitor. Thus, the efficacy of this approach in vivo was tested. To assess the tolerability of dietary serine/glycine limitation with PH755 treatment, the response to the various treatments in a cohort of tumor-free immunocompetent C57BL/6J mice was tested. Mice moved to the −SG diet showed a slight drop in weight that stabilized over the course of the study (FIG. 44). Treatment with PH755 alone did not result in any detectable adverse response in these mice, which did not lose body weight compared to control mice (FIG. 44). However, mice cotreated with PH755 and the −SG diet showed greater weight loss compared to either treatment alone (FIG. 44), despite remaining active and appearing healthy. The weight loss was highly responsive to the dose of PH755, and modulation of the dose (from 75 to 50 mg/kg) was successful in limiting weight loss to less than 20% over the course of the study.

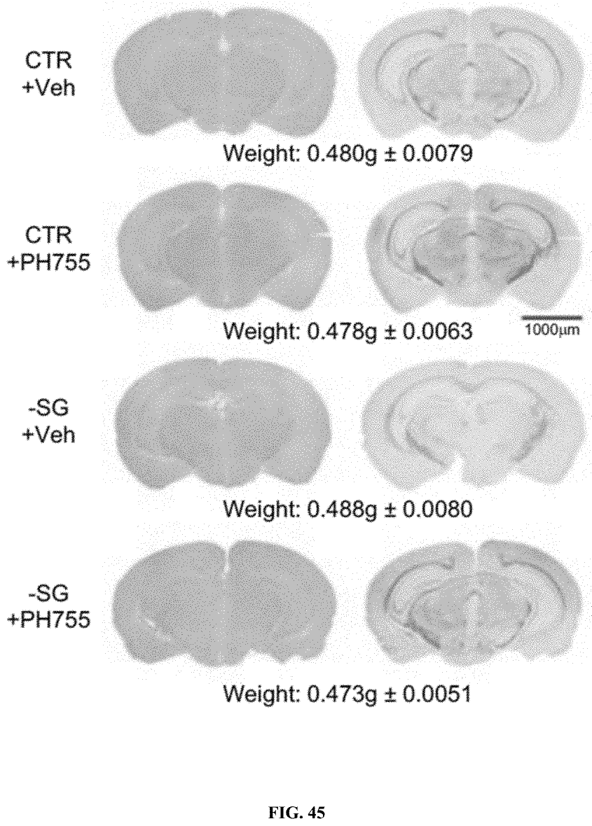

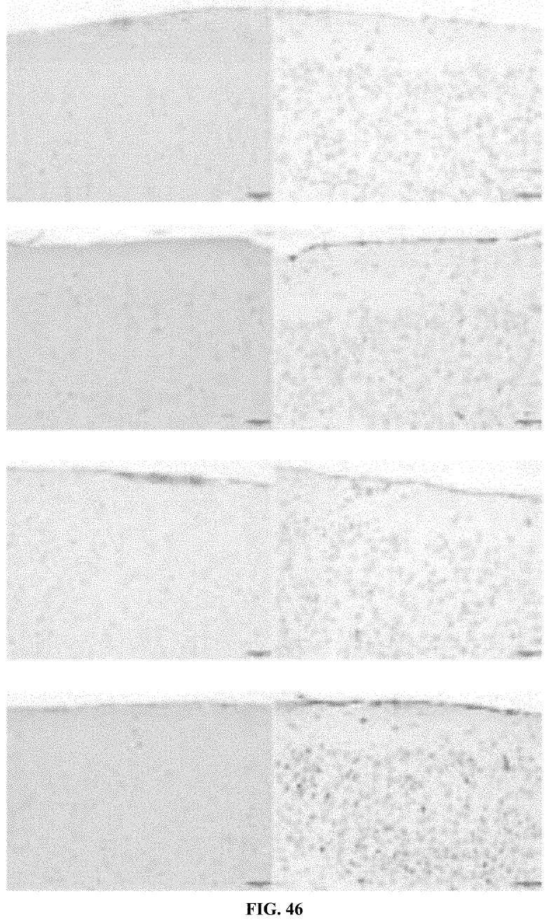

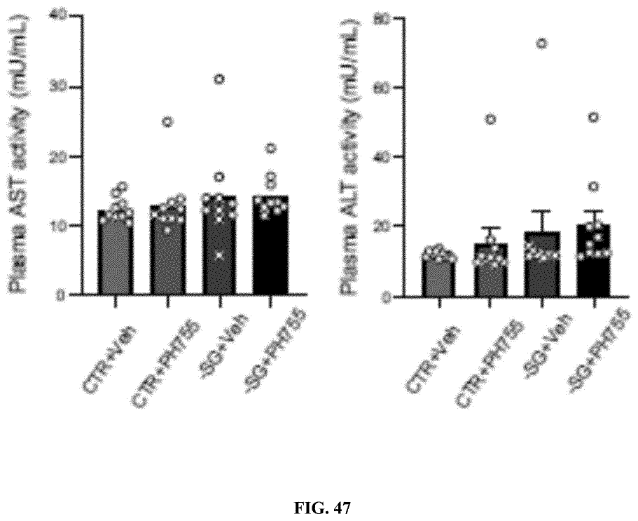

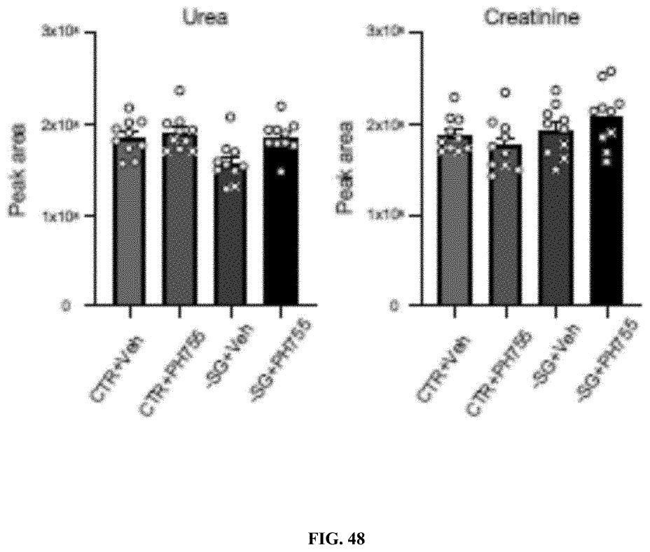

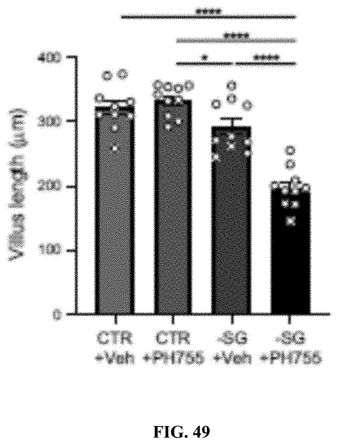

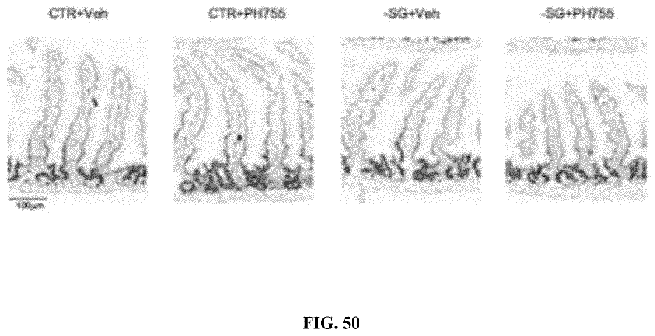

Serine is important in brain development and function and PHGDH deficiency in humans can lead to neurological defects such as microcephaly, psychomotor retardation, and seizures. The impact of −SG diet and PHGDH inhibitor treatment on the brain morphology of a cohort of C57BL/6J mice after 20 days of treatment was assessed. Microscopic examination of coronal sections from the brains of the 4 groups of mice at the level of the pyriform cortex, caudal diencephalon, caudal mesencephalon, and rostral cerebellum did not reveal any histopathological lesions in any of the sections examined. Indeed, hematoxylin & eosin stained sections exhibit normal histological features with no evidence of degeneration, necrosis or inflammation (FIG. 45 and FIG. 46). Furthermore, the brain weight remained unchanged in all groups of mice (FIG. 45). Other signs of toxicity of the double treatment in these normal mice were looked for. Measurement of plasma AST and ALT activity at end point did not reveal any significant elevation of these markers of liver toxicity in the group of mice treated with −SG diet and PH755 (FIG. 47), while plasma urea and creatinine levels remained normal in the double-treated mice, suggesting that there was no kidney damage (FIG. 48). The only clear deleterious effect of the double treatment in mice was weight loss, and in vitro work using normal mice organoids derived from small intestine revealed that the combination treatment altered their ability to grow (FIG. 18). While serine and glycine starvation or PHGDH inhibition alone had no detectable effect on gut morphology, a significant decrease in the length of intestinal villi in animals co-treated with the −SG diet and PH755 (FIG. 49) was observed, consistent with the greater weight loss seen in these mice. However, these mice showed no clear defect in crypt proliferation—assessed by Ki-67 staining—(FIG. 50), suggestive of relatively unperturbed crypt homeostasis. These results are consistent with the observation that reduction in the dose of PHGDH inhibitor stops further weight loss and suggest that short term combination treatment does not cause long term damage.

### Example 7: Combining a Serine/Glycine-Free Diet and a PHGDH Impedes Tumor Growth In Vivo

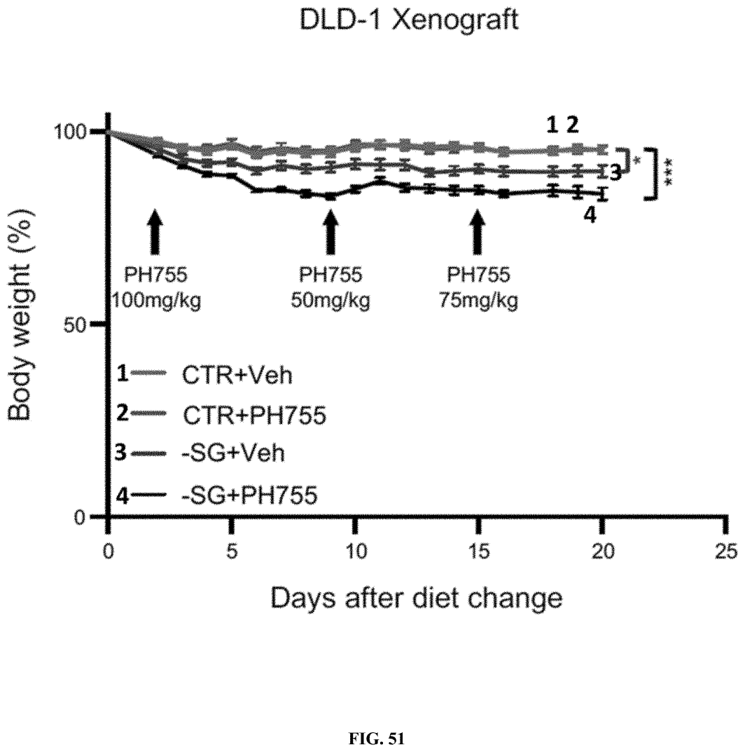

To explore the antitumor efficacy of the combination therapy, xenograft models with two of the colon cancer cell lines that had been tested in vitro, DLD-1 and HCT116, were used. Following subcutaneous injection of cells, mice were transferred to a −SG or control diet when tumors started to become evident and treated with PH755 two-four days later. As seen in the non-tumor bearing mice, the double-treated DLD1 tumor-bearing mice showed more weight loss compared to either treatment alone but a careful modulation of the dose of PH755 used in association with the −SG diet was able to limit the weight loss in these mice to less than 20% over the course of the experiment (FIG. 51).

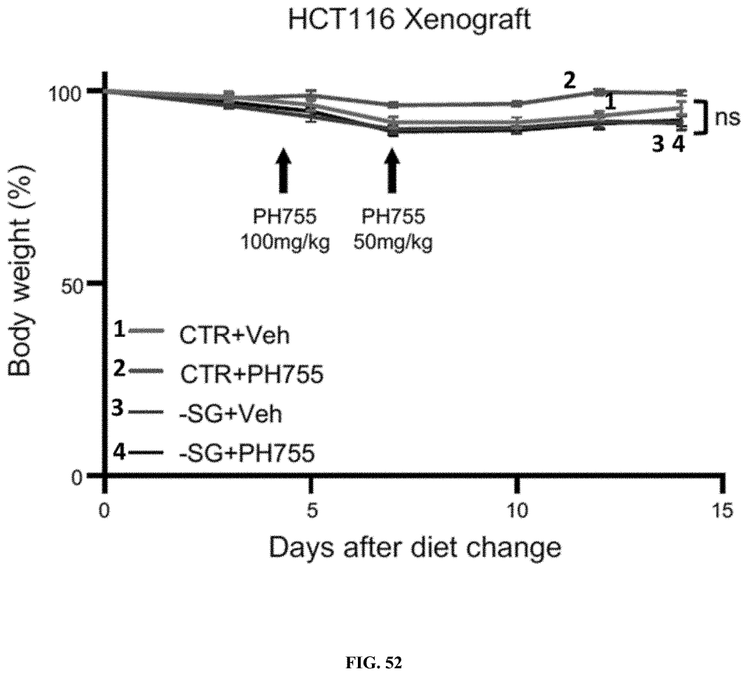

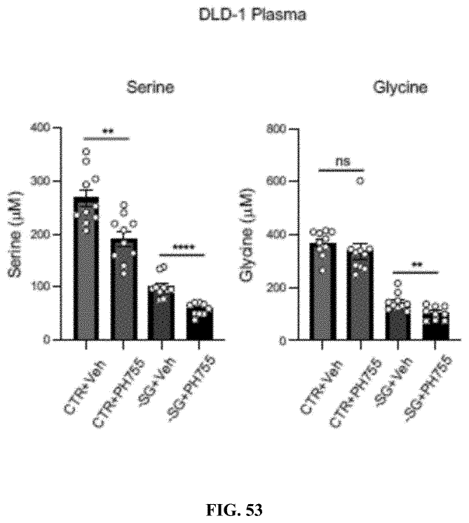

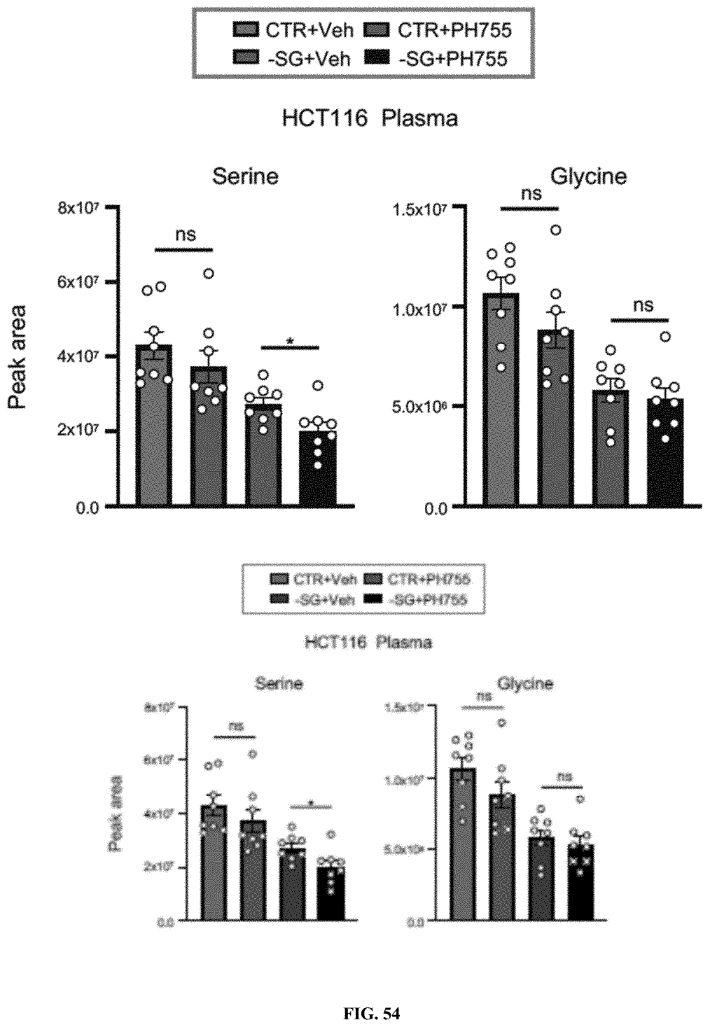

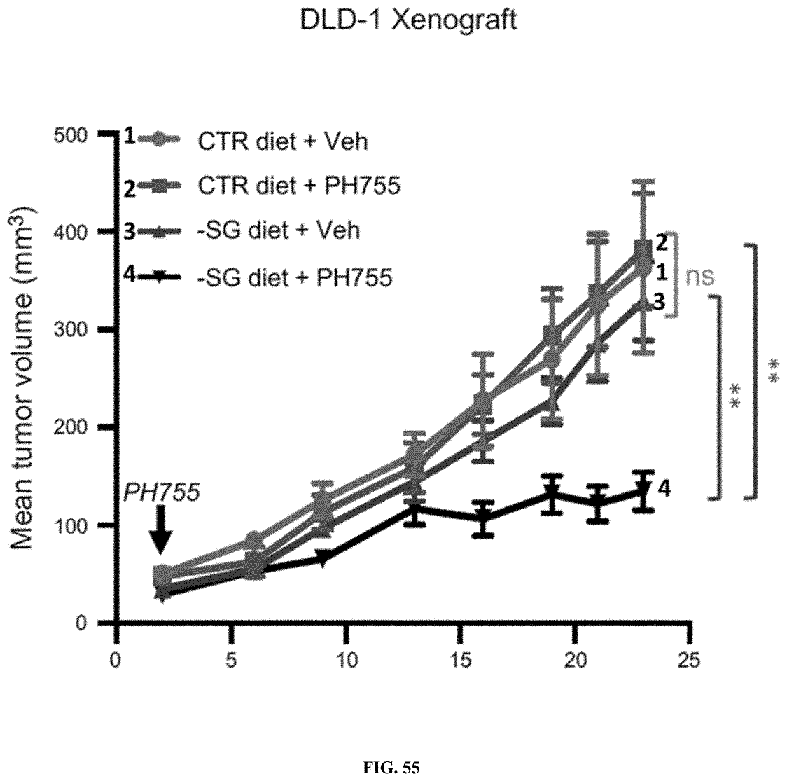

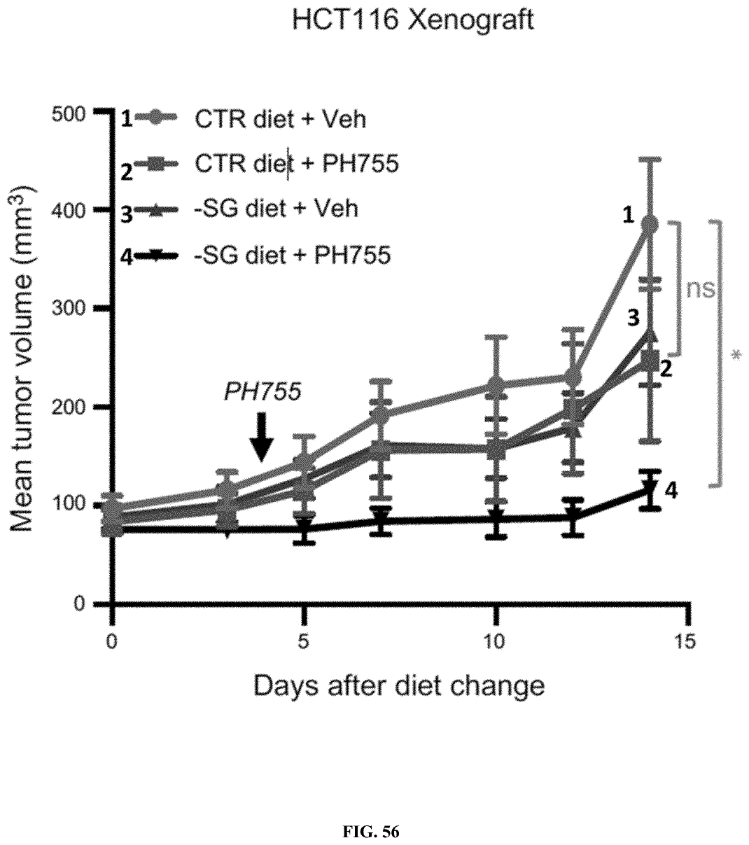

This enhanced weight loss was avoided by increasing the time between diet change and PH755 treatment from 2 to 4 days in the HCT116 experiment (FIG. 51 and FIG. 52). Analysis of the circulating amino acid levels at the end point of the studies confirmed previous observations that the −SG diet resulted in a decrease in plasma serine and glycine levels (FIG. 53 and FIG. 54). While treatment of mice with PH755 had a more modest effect on circulating serine and glycine, a combination of the −SG diet and PH755 most effectively lowered plasma serine and glycine levels reaching absolute concentration as low as 58 μM serine (versus 267.7 μM in control mice and 99.9 μM in mice fed a −SG diet only) and 102.3 μM glycine (versus 367.6 μM in control mice and 143.6 μM in mice fed a −SG diet only) (FIG. 53 and FIG. 54). The growth of tumors arising from DLD-1 cells was not affected by dietary intervention or PH755 treatment alone (FIG. 55), consistent with the lack of effect of either of these treatments on the proliferation of these cells in vitro (FIG. 2). However, a combination of diet and PH755 strongly inhibited the growth of these tumors (FIG. 55). The growth of HCT116 xenograft tumors was somewhat sensitive to dietary serine and glycine restriction and also showed a trend to a decrease in mice treated with PH755 (FIG. 56), consistent with a previous report showing an effect of PH755 on HCT116 tumor growth 24. However, the combination treatment of diet and PH755 almost completely blocked the growth of these tumors (FIG. 56).

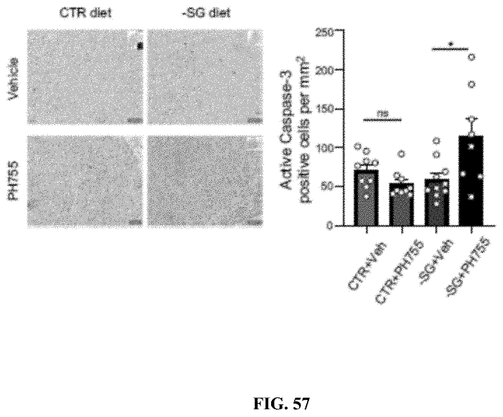

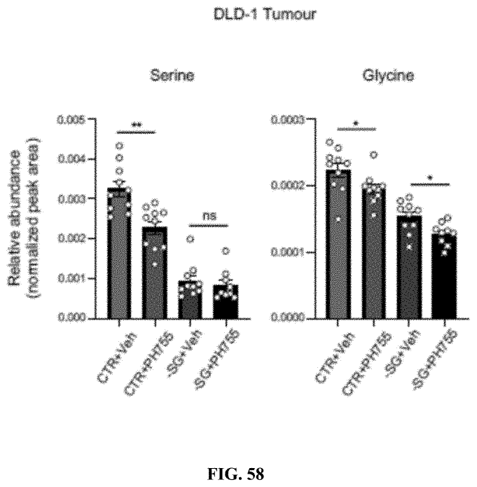

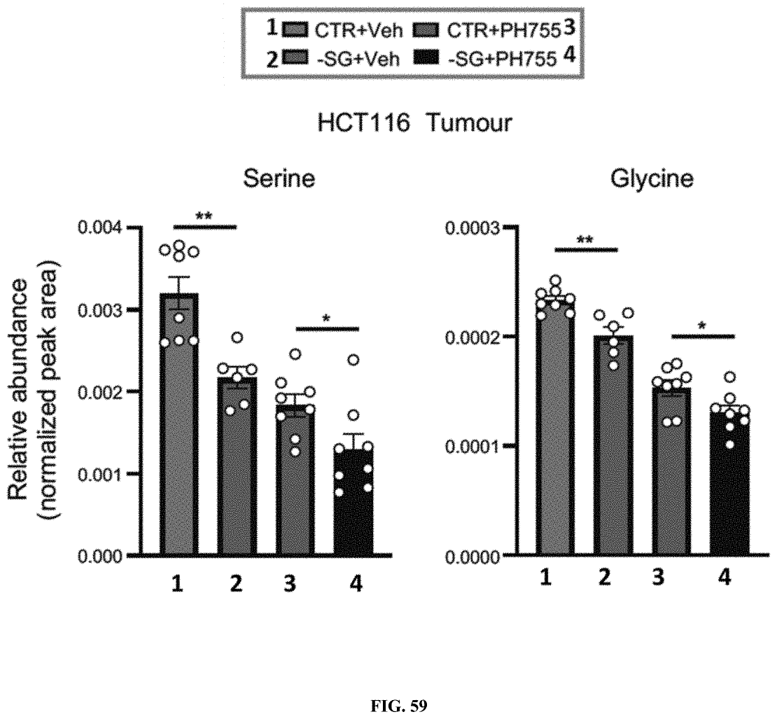

Interestingly, the strong growth inhibition observed in the double treated tumors was accompanied by an increased cell death, as reflected by an increased number of active caspase-3 positive cells in DLD-1 tumors treated with the combination therapy (FIG. 57). Analysis of the serine and glycine levels in the tumors from these mice mirrored the results from the plasma, showing either PH755 treatment or −SG diet lowered intra-tumoral serine and glycine levels (FIG. 58 and FIG. 59), although in each case the −SG diet was more effective in lowering intra-tumoral serine and glycine levels that treatment with the PHGDH inhibitor. HCT116 tumors showed a modest further drop in serine and glycine in the combination diet and drug treated mice (FIG. 59) but in DLD1 tumors, the reduction in serine in response to the −SG diet was not further affected by additional PH755 treatment (FIG. 58). Nevertheless, a further reduction in intra-tumoral glycine in the double treated mice suggests that flux through the SSP is lower in the double treated tumors and that the maintenance of the low steady state levels of serine may reflect the decrease in growth (and serine consumption) under these conditions (FIG. 58).

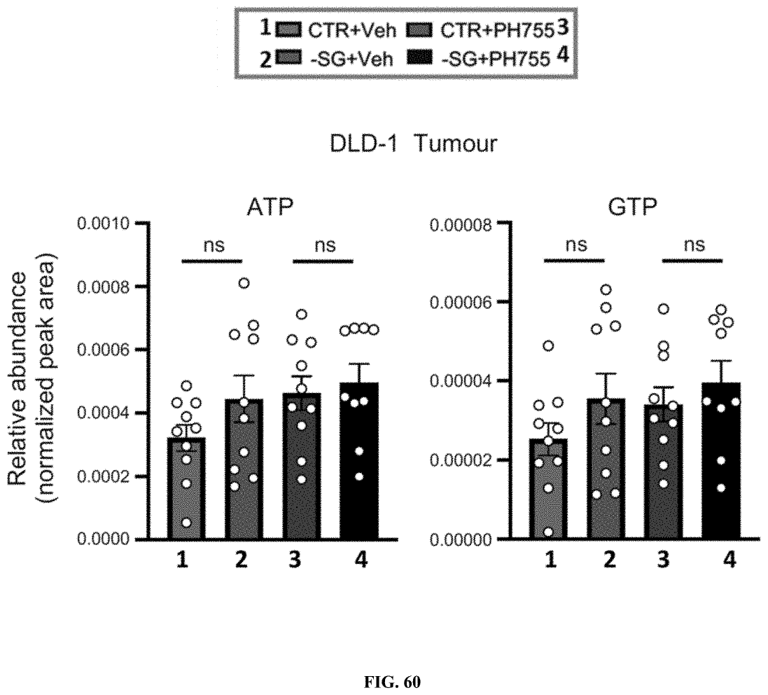

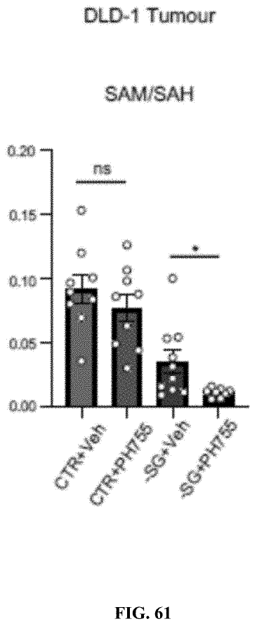

The in vitro data showed that complete inhibition of serine availability through serine starvation and PHGDH inhibition led to defects in one-carbon metabolism and a global inhibition of translation that correlated with a failure to induce an ATF-4 response. To examine these responses to dietary serine/glycine starvation and PHGDH inhibition in vivo, purine levels in the tumors were examined. As noted in vitro (FIG. 25), no difference in total ATP or GTP levels in tumors from double treated mice (FIG. 60) was seen, likely reflecting the decreased proliferation of the double treated tumor cells. In the methionine cycle, the regeneration of SAM from SAH requires one-carbon units. Interestingly, a clear reduction in the SAM/SAH ratio in tumors from −SG diet mice was seen, which was further reduced in mice on −SG diet plus PH755 (FIG. 61). These results are consistent with a defect in one-carbon availability in mice on a −SG diet that is exacerbated in double treated mice.

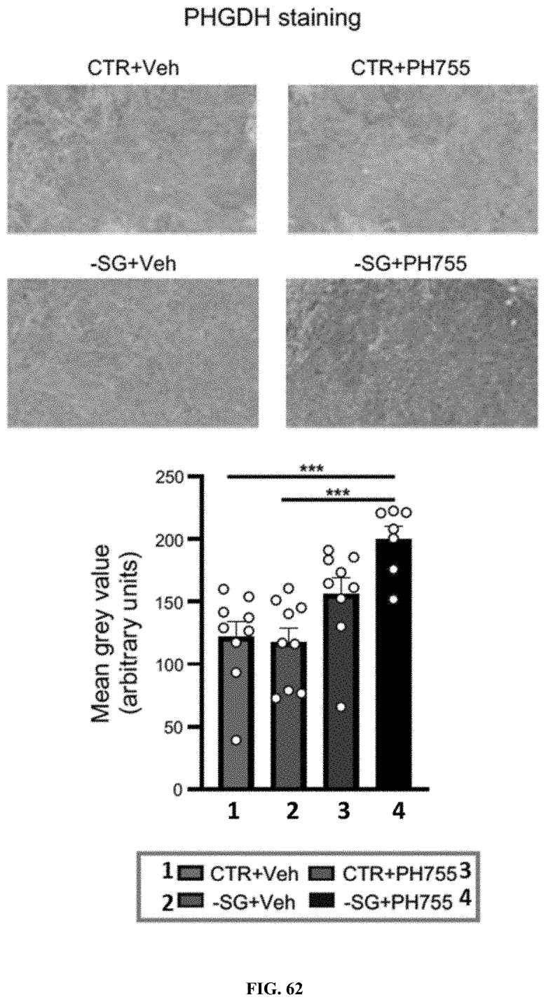

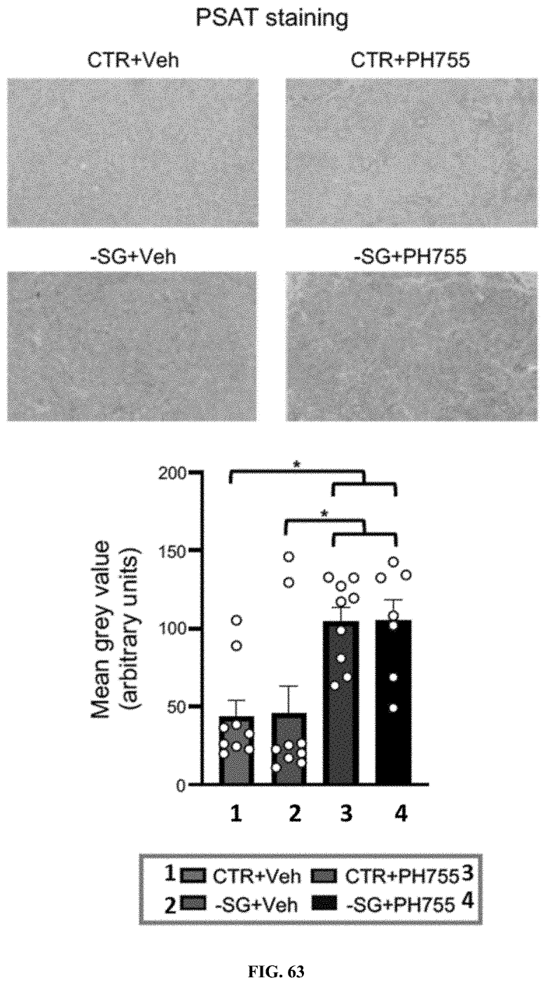

To examine the ATF-4 response, the expression of the two ATF-4 targets, PHGDH and PSAT1, in DLD-1 tumors (FIG. 62 and FIG. 63) was measured. Immunohistochemistry analysis of these tumors revealed that feeding mice with a −SG diet led to a clear induction of PSAT1—and to a lesser extent PHGDH—in tumors, indicating the induction of an ATF-4 response in vivo. By contrast, treating mice with the PHGDH inhibitor alone did not result in any change in PHGDH or PSAT1 expression, suggesting that only dietary restriction of serine and glycine was effective to deplete serine and glycine intratumoral levels enough to lead to an ATF-4 response in vivo. The induction of PSAT-1 and PHGDH was equivalent or even more pronounced in the double-treated tumors compared to the tumors from mice fed a −SG diet only, showing that, in vivo, the combination treatment did not reduce available serine sufficiently to compromise the ability of tumor cells to induce an ATF-4 response (FIG. 62 and FIG. 63). Taken together, these data show that the inhibition of tumor growth correlate with defects in serine metabolism, rather than inhibition of translation.

### Example 8: Combining Dietary Restriction of Serine and Glycine and PHGDH Inhibition Cooperates to Lower Tumor Burden and Improve Survival in Genetic Models of Intestinal Cancer

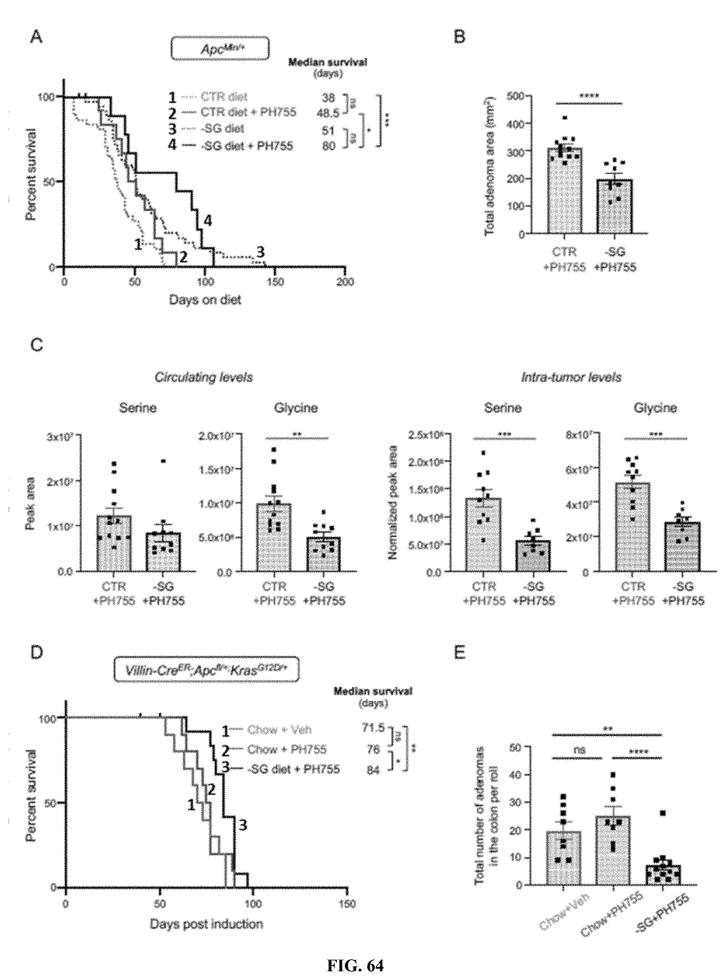

FIG. 64A: ApcMin/+ mice were transferred to a CTR diet (red line) or a −SG diet (black line) at 80 days and were subsequently treated at 84 days with 100 mg/kg PH755 daily for 9 days. After stopping treatment, mice were maintained either on a CTR diet or a −SG diet until clinical end point was reached. These data are shown in comparison to data showing survival of ApcMin/+ mice on control (dotted line) or −SG diet (dotted line). Survival was calculated from change of diet. CTR: n=37; −SG n=35; CTR+PH755: n=12; −SG+PH755 n=12 (ns: no significance, *P<0.05; ***P<0.001; Mantel-Cox test).

FIG. 64B. Total adenoma area measured at clinical end-point in small intestine from ApcMin/+ mice treated with PH755 and fed either a CTR diet or a −SG diet. Data are presented as mean±SEM. CTR+PH755 n=12; −SG+PH755 n=9. (****P<0.0001, unpaired two-tailed Student t-test).

FIG. 64C. Plasma (left panel) or intestinal tumors (right panel) were taken at time of sacrifice from ApcMin/+ mice treated with PH755 and fed either a CTR diet or a −SG diet. LC-MS analysis was performed to evaluate serine and glycine content. Data are presented as mean±SEM. Plasma: CTR+PH755 n=12; −SG+PH755 n=10. Tumors: CTR+PH755 n=10; −SG+PH755 n=8. (** p<0.01; ***P<0.001, unpaired two-tailed Student t-test).

FIG. 64D. Villin-CreER;Apcfl/+;LSL-KrasG12D/+ mice were induced with Tamoxifen at 6-8 weeks of age, then left on normal chow or moved to a −SG diet 2 weeks post induction. 14 days after moving to −SG diet, mice were treated with either 100 mg/kg PH755 for 7 days before moving to 50 mg/kg for 2-12 days, or 50 mg/kg PH755 for 30 days. After stopping drug treatment, mice were then maintained on the respective diets until they reached humane end point. Survival was calculated from time of induction. CTR+Veh: n=10; CTR+PH755 n=10; −SG+PH755 n=14. (ns: no significance, *P<0.05; **P<0.01; Mantel-Cox test).

FIG. 64E. Total number of adenomas scored from H&E staining in the colon per roll in Villin-CreER;Apcfl/+;LSLKrasG12D/+ mice fed a chow diet or a −SG diet and treated with vehicle or PH755. Data are presented as mean±SEM. CTR+Veh: n=8; CTR+PH755 n=8; −SG+PH755 n=12. (** p<0.01; ****P<0.0001, unpaired two-tailed Student t test).

### Example 9: Methods Used in Previous Examples

Cell Culture

All cell lines underwent routine quality control, which included mycoplasma detection, STR profiling and species identification for validation. Cells were cultured at 37° C. in a humidified atmosphere of 5% CO2. HT-29, SW48, SW480, SW620, CACO2, HCT116, RKO, VACO5 and MDA-MB-468 cells were cultured in DMEM supplemented with 10% FBS; DLD-1, HCT-15 and SW1417 cells were cultured in RPMI-1640 medium supplemented with 10% FBS and LoVo and CL-34 cells were cultured in DMEM/F-12 (Gibco, 11320) supplemented with 10% FBS.

Serine and Glycine Deprivation

For all serine and glycine-deprivation experiments, cells were cultured in MEM supplemented with 10% dialyzed FBS, 1% penicillin-streptomycin, D-glucose (5 mM), sodium pyruvate (65 μM), 1×MEM vitamin solution (Gibco, 11120), L-Glutamine (2 mM), L-Proline (0.15 mM), L-Alanine (0.15 mM), L-Aspartic acid (0.15 mM), L-Glutamic acid (0.15 mM) and L-Asparagine (0.34 mM) (−SG media). The complete medium (CM) corresponds to the previously described medium supplemented with 0.4 mM L-Serine and 0.4 mM L-Glycine.

Growth Curves

Cells (2×10⁴ to 3×10⁴ cells/well depending on the cell lines) were plated in 24-well plates in their regular medium. The next day, after being washed with PBS, cells were transferred to −SG medium or CM and treated with 10 μM PH755 diluted in DMSO or DMSO alone. For the counting step, cells were trypsinized, suspended in PBS-EDTA, and counted with a CASY Model TT Cell Counter. Relative cell number at each time point was calculated based on the number of cells measured before the medium change. For the growth curve experiment with formate and glycine supplementation, HT-29, HCT116 and DLD-1 cells were seeded in 24-well plates (2×10⁴ cells/well). Sodium formate (1 mM) and/or glycine (0.4 mM) were diluted in −SG medium+10 μM PH755 and medium was refreshed every two days.

Organoids

Crypts were isolated from adenomatous small intestine tissue derived from Vil1-creER;Apcfl/fl and Vil1-creER;Apcfl/fl;KrasG12D/+ mice. The generation of the Apc5 organoid bearing an Apc truncating mutation using CRISPR/Cas9 technology and isolation of normal organoids derived from the proximal part of healthy small intestine from Villin-CreERT2 mouse were performed. Cancer organoids from mice were cultured in tumor organoid medium (CM) composed of Advanced DMEM/F12 supplemented with 1% penicillin-streptomycin solution, 0.1% BSA, 2 mM L-glutamine, 10 mM Hepes, 50 ng/mL EGF, 100 ng/mL Noggin, 500 ng/mL Spondin, 1×N-2 Supplement and 1×B-27 Supplement (ThermoFisher 17504044). The −SG medium corresponds to the previously described medium without serine and glycine. Normal organoids from mice were grown in normal organoid medium, a modification of tumor organoid medium that was supplemented with 100 ng/mL Wnt-3a, 1 mM N-Acetyl-L-cysteine, 10 μM Y 27632 and 4 mM Nicotinamide.

Human organoids were grown in human organoid medium, a second modification of tumor organoid medium that was supplemented with 10 nM FGF-basic, 100 ng/mL Wnt-3a 1 μM Prostaglandin E2, 4 mM Nicotinamide, 20 ng/mL HGF, 10 nM FGF-10, 10 nM Gastrin I, 10 μM Y-27632, 0.5 μM A 83-01 and 5 μM SB 202190.

For the splitting step, organoids were harvested through mechanical pipetting using TrypLE, incubated for 10 minutes at 37° C., diluted 3 times in volume in ice-cold 1×HBSS, and spun down at 270 g for 5 minutes at 4° C. Pellet was then resuspended in growth factor reduced Matrigel and plated in 24-well plates. Matrigel was then incubated for 15 min at 37° C. and 1 mL of the CM described above was added. The next day, organoids were washed with PBS and the medium was replaced with CM or −SG medium supplemented or not with 10 μM PH755 and allowed to grow. Pictures were regularly taken with a light microscope and organoid diameter was measured using ImageJ software.

Generation of PHGDH KO Cells

pLentiCRISPRv2 vector containing the following guide RNA: TGGACGAAGGCGCCCTGCTC (SEQ ID NO: 1) was used to target PHGDH. HEK293T cells were transfected with this lentiviral plasmid together with psPAX2 and VSV.G using jetPRIME reagent (Polyplus transfection). After 24 hours incubation, medium was changed and 48 hours later, the viral particle containing-medium was filtered (0.45 mm) and mixed with polybrene (4 μg/ml, Sigma-Aldrich). The medium containing lentiviruses was incubated with the target cells for 24 hours. HT-29 and DLD-1 cells were then selected with puromycin for 3 weeks and analyzed for loss of PHGDH expression.

ATF-4 siRNA Transfection

Cells were transfected with siRNA using Lullaby transfection reagent.

BrdU/7-AAD Staining

HCT116 and DLD-1 cells were grown for 48 hours in −SG medium or CM and treated with 10 μM PH755 diluted in DMSO or DMSO alone. To determine the percentage of bromodeoxyuridine (BrdU) positive cells, 10 μM BrdU was then added to culture media for an additional 5 hours while for cell cycle analysis, 10 μM BrdU was added for only 30 minutes. Cells were then harvested, fixed and stained with APC anti-BrdU antibody (and 7-AAD for cell cycle analysis) using the APC BrdU Flow kit. Fluorescence was acquired with FACSdiva on a Fortessa flow Cytometer and the analysis performed using FlowJo (version 10.5.2).

Western Blot

Protein lysates were processed in RIPA-buffer supplemented with phosphatase inhibitor cocktail and complete protease inhibitors. Lysates were separated using precast NuPAGE 4-12% Bis-Tris Protein gels and transferred to nitrocellulose membranes. Following incubation with primary antibodies, appropriate secondary antibodies were used to detect the proteins. Westerns scanned using the Odyssey CLx or visualized using ECL chemiluminescence detection kits.

Primary antibodies used were as follows: PHGDH (13428), ATF-4 (11815), Phospho-eIF2α (Ser51) (3398), Phospho-p70S6 kinase (Thr389) (9234), p70S6 kinase (9202), c-Myc (5605), HIF-1a (14179), Caspase-3 (9662), Cleaved Caspase-3 (Asp175) (9661), beta-Actin (4970); GCN2 (sc-374609), eIF2α (sc-133132), p53 (sc-126), Vinculin (sc-73614); PSAT (ab96136), PSPH (ab96414), Phospho-GCN2 (Thr899) (ab75836); ASNS (HPA029318) from Atlas Antibodies; Puromycin (MABE343). All primary antibodies were diluted at 1:1000 dilution.

Protein Synthesis and Degradation

Cells were grown for 24 hours in −SG medium or CM and treated with 10 μM PH755 diluted in DMSO or DMSO alone. To evaluate protein synthesis, puromycin (final concentration: 90 μM) was added to each well 10 minutes prior harvesting the cells for western blot analysis, except in the negative control well. Where indicated, cells grown in CM medium were treated with cycloheximide (10 μg/mL) for the last 5 hours providing a control for translation inhibition. Incorporation of puromycin into newly synthesized proteins was assessed by Western blot using an anti-puromycin antibody. To assess the accumulation of short-lived proteins in response to proteasome inhibition, cells grown in −SG medium or CM plus or minus 10 μM PH755 for 24 hours were treated for the last 6 hours with the proteasome inhibitor MG-132 (10 μM) before harvesting the cells for western blot analysis.

qPCR

HT-29, HCT116 and DLD-1 cells were grown for 6 hours or 24 hours in −SG medium or CM and treated with 10 μM PH755 diluted in DMSO or DMSO alone. Total RNA was extracted using RNeasy Mini kit performing on-column digestion of DNA and reverse transcribed using the High-Capacity cDNA Reverse Transcription kit. qPCR was performed using PrimeTime Gene Expression Master Mix with the primers listed TABLE 1 below.

TABLE 1

| Gene | Primer 1 | Primer 2 | Probe |
|---|---|---|---|
| ASNS | AGTACAGTATC | TCACTTCCAAT | TTCTAGCAGCCA |
| | CTCTCAGACA | ATGATCTGCCA | GTAAATCGGGGC |
| | (SEQ ID NO: 2) | (SEQ ID NO: 3) | (SEQ ID NO: 4) |
| ATF4 | AGGTGTCTTTG | CGTATTAGGG | CCATGGCGCT |
| | TCGGTTACAG | GCAGCAGTG | TCTCACGGC |
| | (SEQ ID NO: 5) | (SEQ ID NO: 6) | (SEQ ID NO: 7) |
| PHGDH | CACTGAGGCT | GTCATCAACG | CCAGATCCACAT |
| | GTTCCCATT | CAGCTGAGAA | TGTCCACACCTG |
| | (SEQ ID NO: 8) | (SEQ ID NO: 9) | (SEQ ID NO: 10) |
| PSAT | TCATCACGGA | GTCCTCAAACT | AGAGCCAACATT |
| | CAATCACCAC | TCCTGTCCAA | CTTCTGGGCACC |
| | (SEQ ID NO: 11) | (SEQ ID NO: 12) | (SEQ ID NO: 13) |
| PSPH | CATGATTGGA | TTATCCTTGAC | TGTCCTCCTGCTG |
| | GATGGTGCCA | TTGTTGCCTGA | ATGCTTTCATTGG |
| | (SEQ ID NO: 14) | (SEQ ID NO: 15) | (SEQ ID NO: 16) |
| ACTB | CCTTGCACA | ACAGAGCCT | TCATCCATGGT |
| | TGCCGGAG | CGCCTTTG | GAGCTGGCGG |
| | (SEQ ID NO: 17) | (SEQ ID NO: 18) | (SEQ ID NO: 19) |

The QuantStudio 7 Flex Real-Time PCR System was used for all reactions. Gene expression was normalized to ACTB (b-actin) housekeeper gene, analyzed according to Pfaffl method and expressed as relative units compared to the cells grown in CM for 6 hours.

Liquid Chromatography-Mass Spectrometry

HT-29 cells (2.4×10⁵), HCT116 cells (1.8×10⁵), DLD-1 cells (1.8×10⁵) and MDA-MB-468 cells (2.4×10⁵) were plated in 6-well plates in their regular medium. Duplicate plates were used for cell counting to normalize LC-MS analysis based on cell number. After 16 hours, cells were washed with PBS and transferred to CM or −SG medium supplemented or not with 10 μM PH755 for 24 hours. 6 hours before metabolite extraction, medium was replaced with CM or −SG medium+/−10 μM PH755 with glucose substituted for 10 mM U-[¹³C]-glucose. For short-term experiments, cells were moved to the previously described medium with glucose substituted for 10 mM U-[¹³C]-glucose for only 3 hours or 6 hours before metabolite extraction. For measurement of glycine conversion into serine during rescue experiment, cells were grown for 24 hours in −SG medium supplemented with 10 μM PH755, 1 mM sodium formate and 0.4 mM glycine. This medium was then replaced with matched medium with glycine substituted for 0.4 mM ¹³C₂¹⁵N₁-glycine for 1 hour before metabolite extraction.

For half of the samples, a pulse of 1 mM unlabeled serine was added to the medium 1 minute before metabolite extraction to allow labelled serine to accumulate. Cells were then washed with PBS and metabolites were extracted using ice-cold extraction buffer composed of methanol, acetonitrile, and H₂O in the following ratio 50:30:20. For LC-MS analysis on tumor samples, tissue was homogenized (20-40 mg tissue/mL of the previously described extraction buffer) using the Precellys 24 homogenizer. Samples were spun (16,000 g/10 minutes/0° C.) and the supernatant collected to be centrifuged again (16,000 g/10 minutes/0° C.). Supernatant were then collected for LC-MS analysis.

For LC-MS analysis on mice plasma, plasma was diluted 20-50-fold with the same extraction buffer, vortexed for 30 seconds and centrifuged (16,000 g/10 minutes/0° C.). Supernatant were then collected for analysis. Absolute levels of serine and glycine in plasma was determined using 8-point calibration curves (from 2.5 to 800 μM) with ¹³C₃¹⁵N₂-serine and ¹³C₂¹⁵N₁-glycine diluted in plasma. LC-MS analysis was performed.

In Vivo Experiments

Mice (3 to 5 per cage) were allowed access to food and water ad libitum and were kept in a 12-hour day/night cycle starting at 7:00 until 19:00. Rooms were kept at 21° C. at 55% humidity. Mice were allowed to acclimatize for at least one week prior to the experiment. They were then randomly assigned to experimental groups. The experimental diets used in this study (control diet and −SG diet) were described as “Diet 1-Control” and “Diet 1−SG-free”. Briefly, the control diet contained all essential amino acids as well as serine, glycine, glutamine, arginine, cystine, and tyrosine. The −SG diet was the same as the control diet but was deprived of serine and glycine, which were compensated by a proportionally increased level of the other amino acids to reach the same total amino acid content.

Xenograft Experiments

CD-1 female nude mice (7-9 weeks old) received unilateral subcutaneous injections of 100 μl of HCT116 cells (2×106 cells) or 100 μl of DLD-1 cells (4×10⁶ cells) suspended in PBS.

Mice were placed on experimental diets (control or −SG) 10 days (for HCT116 xenograft experiment) or 2 days (for DLD-1 xenograft experiment) after tumor injections. 4 days (for HCT116 xenograft experiment) or 2 days (for DLD-1 xenograft experiment) after the diet change, mice were treated either with vehicle (0.5% methylcellulose, 0.5% Tween-80) or PH755 prepared in vehicle once daily by oral gavage. The starting dosage of PH755 was 100 mg/kg and was subsequently lowered to 75 mg/kg or 50 mg/kg as indicated in the figure legends. Subcutaneous growth was measured two to three times a week by caliper and the following formula: (length×width²)/2 was used to calculate tumor volume.

C57BL/6J Experiment

C57BL/6J male mice (14 weeks old) were placed on experimental diets (control or −SG) two days before stating the treatment with PH755 or its vehicle. Mice were treated once daily by oral gavage with PH755 or its vehicle for 20 days. The starting dosage of PH755 was 75 mg/kg and was subsequently lowered to 50 mg/kg to maintain weigh loss below 20% of the initial body weight.

Immunohistochemistry

All tissues were fixed in 10% neutral buffered formalin and were embedded in paraffin. For PHGDH and PSAT1 staining, the slides were de-paraffinized in xylene and rehydrated using a series of graded industrial methylated spirits solutions and distilled water. Antigen retrieval was performed for 23 minutes in the microwave using pH 6 0.1 M citrate buffer. Endogenous peroxidase blocking was performed using 1.6% H₂O₂ for 10 minutes at room temperature and protein blocking was performed using 2.5% Normal Horse Serum (MP-7401, Vector) overnight at 4° C. Primary antibody was diluted at 1:1000 for PHGDH antibody (HPA021241) and at 1:500 for PSAT1 antibody (PA5-22124) in 1% BSA, and incubated for 1 hour at room temperature. HRP Horse Anti-Rabbit IgG Polymer (MP-7401, Vector) was incubated for 30 min at room temperature. 3,3-diaminobenzidine (DAB) chromogen (SK-4100, Vector) was incubated for 10 minutes at room temperature. The slides were counterstained with Harris Hematoxylin, dehydrated, cleared and mounted in a Sakura Tissue-Tek PrismaR auto stainer. For PHGDH and PSAT1 staining intensity quantification, a minimum of 3 fields per tumor were quantified with ImageJ. Active Caspase-3 immunohistochemistry was performed on the Discovery Ultra Ventana platform.

Antigen retrieval was obtained with Cell Conditioning 1 (CC1) from Ventana Medical Systems. Primary antibody (AF835) was diluted at 1:1250 and incubated for 60 minutes. For Active Caspase-3 staining, a minimum of 3 fields per tumor were quantified with the positive cell detection algorithm from QuPath (version 0.1.2). All slides were scanned with the ZEISS Axio Scan.Z1 slide scanner and images were generated through ZEISS ZEN 2.6 (blue edition) software. For gut rolls, immunohistochemistry was performed on Bond Rx Autostainer Leica Bond Intense R staining kit. Slides were de-paraffinized with Bond Dewax at 72° C. for 30 min and antigen retrieval was achieved with ER2 at 100° C. for 20 min. Primary antibody (Ki67 SP6, ab16667) was diluted at 1/100 and incubated for 35 min. For villus length measurement, villi from the same area of the small intestine (at least 15 per mouse) were measured from the crypt/villus junction to the villus tip, using ImageJ.

Brain Sampling and Pathological Examination

C57BL/6J mice were culled using carbon dioxide asphyxiation to avoid physical trauma to the brain. Mice were immediately dissected, and haired skin and soft tissue were removed from the cranial surface. Incisions throughout the parietal and frontal sutures were performed to allow fast penetration of the fixative solution into the brain parenchyma. The head was immersed in 250 mL of 10% neutral buffered formalin and fixed for 2 weeks. After complete fixation, the brains were removed from the skull and trimmed using a mouse brain matrix (BSMYS001-1). Four coronal sections were obtained at the level of the pyriform cortex, caudal diencephalon, caudal mesencephalon and rostral cerebellum. Tissue samples were routinely processed for paraffin embedding, sectioned at 4 and stained with hematoxylin and eosin.

Histopathological examination of brains was performed by a board-certified veterinary pathologist.

Blood Biochemical Marker Assays

Plasma ALT and AST activities were measured using Alanine Transaminase Activity Assay Kit (and AST Activity Assay Kit respectively.

Statistical Analyses

All data are expressed as mean±SEM and each statistical analysis is detailed in the figure legend. Data were collected in Excel (version 16.16.26) and all statistical analyses were performed using GraphPad Prism 8 (version 8.3.1) software. Unpaired Student t test was performed to compare two groups to each other. If the variance between the two groups was unequal, a Welch's correction was applied.

To compare more than two groups, statistical significance was determined using one-way ANOVA with Tukey's multiple comparison test. For tumor volume and body weight analyses, two-way ANOVA plus Tukey's post hoc test were performed. p value below 0.05 was considered statistically significant.

Significance is indicated as follows: * p<0.05, ** p<0.01, *** p<0.001, **** p<0.0001, ns: no significance. All measurements were taken from distinct samples. Sample sizes were based on standard protocols in the field and the metabolic samples were assigned in a random order before analysis. Mice were randomly assigned to a treatment and the identity of each mouse was blinded when measurements were collected.

### Example 10: Metabolomic Impact of Radiation on Pancreatic and Colorectal Cancer Cells in Vitro

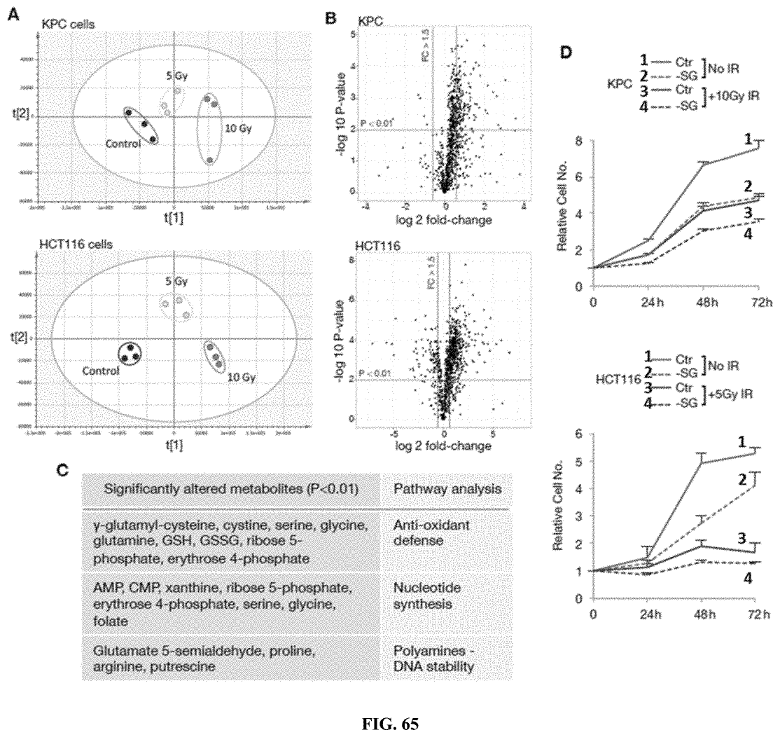

FIG. 65 PANEL A-PANEL D show the metabolomic impact of radiation on pancreatic and colorectal cancer cells in vitro. In PANEL A, primary murine pancreatic cancer (KPC: Pdx1-cre; Kras^{G12D/+}; Trp53^{R172H/-}) and human colorectal cancer (HCT116) cells were exposed to 5-10 Gray (Gy) radiation. After 24 h, metabolites were extracted and analyzed by LCMS using a Thermo Exactive Orbitrap Mass Spectrometer coupled to a pHILIC chromatography column. Unsupervised principle component analysis was performed using data from all identified metabolites. PANEL B shows volcano plots showing distribution of identified metabolites (Control vs. 10 Gy radiation) in terms of fold-change and P-value for KPC and HCT116 cells. PANEL C shows significantly altered metabolites identified during unbiased metabolomics were subjected to metabolic pathway analysis. The dominant pathway hits are shown. PANEL D shows that KPC (Top panel) and HCT116 (Bottom panel) cells were either grown in complete medium (Ctr) or medium lacking serine and glycine (−SG) and irradiated with 10 or 5 Gray radiation (IR), respectively. Cell number over time (hours) is shown. Data are averages of triplicate wells, and error bars are SD.

### Example 11: Effect of Dietary Amino Acid Restriction on Response to Targeted Radiotherapy In Vivo

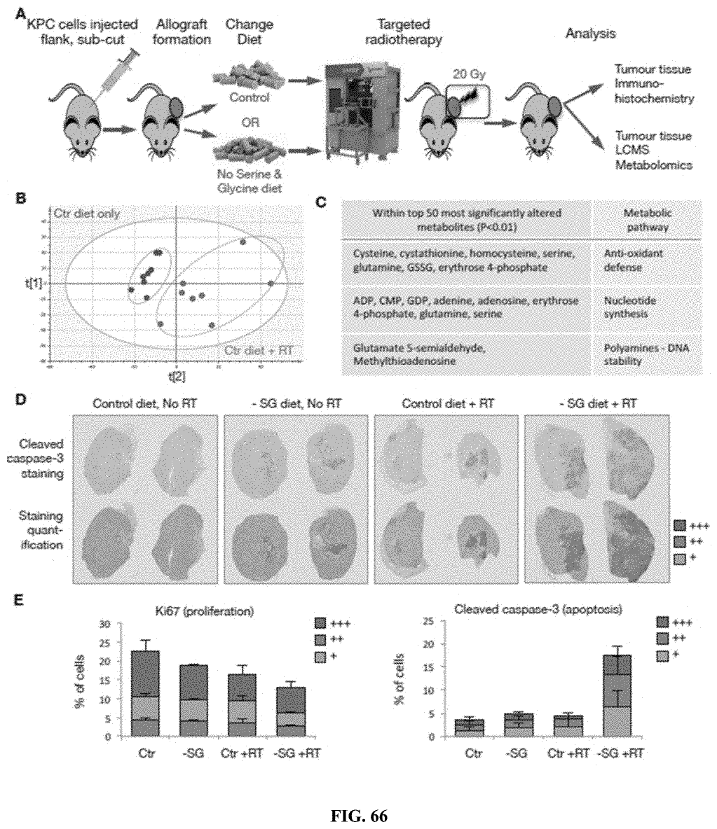

FIG. 66 PANEL A-PANEL E show the effect of dietary amino acid restriction in response to targeted radiotherapy in vivo. PANEL A is a cartoon illustrating the experimental setup of a pilot experiment testing the impact of dietary restriction of serine and glycine on the response of KPC tumors to radiation. Groups of C57BI6 (n=4) mice were injected (subcutaneous, bilaterally) with 2×10\hat{ }6 primary C57BI6 KPC cells. Once tumors had formed, mice were placed on control or serine and glycine-free diets. After 4 days on the diets, mice were anesthetized and positioned in an Xstrahl SARRP. Using high resolution cone-beam CT imaging, a 2 mm focused beam of x-ray radiation (20 Gy) was delivered to each tumor. PANEL B and PANEL C show the results of an unsupervised principle component analysis performed on tumor tissue to assess the metabolic impact of radiotherapy alone. The most significantly altered metabolites in vivo converged on the same metabolic pathways as identified in vitro. PANEL D shows representative tumor cross sections stained for cleaved caspase-3 by immunohistochemistry. The upper panels show stained sections (cleaved caspase-3 stained brown), lower panels show false color mapping of histological scores generated by Halo image analysis software. PANEL E shows quantification of Ki67 and cleaved caspase-3 staining as quantified using Halo image analysis software. n=8 tumors per group, bars are SEM. The data indicate that the cells obtained from mice on the serine and glycine-free diet in combination with radiotherapy showed less proliferation and apoptosis than the other experimental conditions.

### Example 12: Sachet Formulation Devoid of Serine, Glycine, and Proline

A sachet containing a formulation devoid of serine, glycine, and proline is prepared and contains 0.8 g/kg/day of amino acids. TABLE 2 shows the components and amounts of the composition. The amino acid sachet is administered to a subject in conjunction with a low protein and low carbohydrate diet. The low protein and low carbohydrate diet results in a daily dietary intake of: 1) 1711 kcals/day (1923 kcals/day with sachets); 2) about 10 g protein/day; 3) about 420 mg proline/day; 4) about 410 mg/serine/day; 5) about 230 glycine/day; 6) a diet that is about 9% carbohydrates, 2% protein, and 89% fat of food-only kcals.

TABLE 2

| Amino Acids | Chemical Name | Milligrams (mg) |
|---|---|---|
| 1 | L-Histidine | 445.00 |
| 2 | L-Isoleucine | 600.00 |
| 3 | L-Leucine | 1,150.00 |
| 4 | L-Lysine Monohydrate | 995.00 |
| 5 | L-Methionine | 300.00 |
| 6 | L-Phenylalanine | 750.00 |
| 7 | L-Threonine | 600.00 |
| 8 | L-Tryptophan | 220.00 |
| 9 | L-Valine | 600.00 |
| 10 | L-Cystine | 138.00 |
| 11 | L-Tyrosine | 330.00 |
| 12 | L-Glutamine | 300.00 |
| 13 | L-Arginine Base | 246.00 |
| 14 | L-Alanine | 600.00 |
| 15 | L-Aspartic Acid | 508.70 |
| 16 | L-Asparagine Hydrate | 600.00 |
| 17 | L-Arginine-L-Glutamate Salt | 1,300.00 |
| 18 | L-Serine | 0.00 |
| 19 | Glycine | 0.00 |
| 20 | L-Proline | 0.00 |
| 21 | Taurine | 50.00 |
| Total Amino Acids | | 9,732.70 |
| Other Materials | | |
| 22 | L-Aspartic Acid Potassium Salt | 380.00 |
| 23 | L-Aspartic Acid Magnesium Salt | 211.00 |
| 24 | D-Glucose | 0.00 |
| Total Materials | | 10,323.70 |

### Example 13: Use of Radiotherapy to Treat a Cancer

A first subject with a cancer is treated with a short course of radiotherapy to treat the cancer. The first subject is placed on a diet substantially devoid of serine and glycine two days before starting radiotherapy treatment (i.e., day −2). The amino acid-depleted diet is administered for a total of 10 days, starting 2 days before treatment through 4 days post-treatment (i.e., day −2 through day 8). The first subject is treated with 5 Gy a day for 5 days. The first subject returns to a normal, habitual diet after day 8, or 4 days post-radiation treatment. If the first subject is treated with chemotherapy after the radiotherapy, the first subject is placed on a cycled diet throughout the chemotherapy. The cycle diet places the first subject on an alternating 5 day amino acid-depleted diet (e.g., Monday-Friday) followed by a 2 day habitual diet (e.g., Saturday, Sunday) throughout the chemotherapy treatment period. TABLE 3 shows a short course radiotherapy to treat a cancer.

TABLE 3

| Day number | Day of week | Radiotherapy dose | Diet |
|---|---|---|---|
| −3 | Fri | 0 Gy | Habitual |
| −2 | Sat | 0 Gy | AA depleted Day 1 |
| −1 | Sun | 0 Gy | AA depleted Day 2 |
| 0 | Mon | 5 Gy | AA depleted Day 3 |
| 1 | Tue | 5 Gy | AA depleted Day 4 |
| 2 | Wed | 5 Gy | AA depleted Day 5 |
| 3 | Thu | 5 Gy | AA depleted Day 6 |
| 4 | Fri | 5 Gy | AA depleted Day 7 |
| 5 | Sat | 0 Gy | AA depleted Day 8 |
| 6 | Sun | 0 Gy | AA depleted Day 9 |
| 7 | Mon | 0 Gy | AA depleted Day 10 |
| 8 | Tue | 0 Gy | Habitual |
| Chemotherapy | Ongoing | n/a | Cycle 5 days of AA |
| | | | depleted diet + 2 days |
| | | | habitual diet |
| | | | throughout |
| | | | Chemotherapy |
| | | | treatment |

A second subject with a cancer is treated with a long course of radiotherapy to treat the cancer. The second subject is placed on a diet substantially devoid of serine and glycine two days before starting radiotherapy treatment (i.e., day −2). The amino acid-depleted diet is administered for a total of 7 days, starting 2 days before treatment through the course of treatment (i.e., day −2 through day 4). The second subject is treated with 2 Gy a day for 5 days. The second subject returns to a normal, habitual diet for two days before starting an additional round of radiotherapy. Subsequent radiation therapy cycles administer 5 days of an amino-acid depleted diet with 2 Gy of radiation for 5 days, followed by 2 days of a habitual diet. The cycle is repeated as needed.

If the second subject is treated with chemotherapy after the radiotherapy, the second subject is placed on a cycled diet throughout the chemotherapy. The cycle diet places the second subject on an alternating 5 day amino acid-depleted diet (e.g., Monday-Friday) followed by a 2 day habitual diet (e.g., Saturday, Sunday) throughout the chemotherapy treatment period. TABLE 4 shows a long course radiotherapy treatment to treat a cancer.

TABLE 4

| Day number | Day of week | Radiotherapy dose | Diet |
|---|---|---|---|
| −3 | Fri | 0 Gy | Habitual |
| −2 | Sat | 0 Gy | AA depleted Day 1 |
| −1 | Sun | 0 Gy | AA depleted Day 2 |
| 0 | Mon | 2 Gy | AA depleted Day 3 |
| 1 | Tue | 2 Gy | AA depleted Day 4 |
| 2 | Wed | 2 Gy | AA depleted Day 5 |
| 3 | Thu | 2 Gy | AA depleted Day 6 |
| 4 | Fri | 2 Gy | AA depleted Day 7 |
| 5 | Sat | 0 Gy | Habitual |
| 6 | Sun | 0 Gy | Habitual |
| 7 | Mon | 2 Gy | AA depleted Day 1 |
| 8 | Tue | 2 Gy | AA depleted Day 2 |
| 9 | Wed | 2 Gy | AA depleted Day 3 |
| 10 | Thu | 2 Gy | AA depleted Day 4 |
| 11 | Fri | 2 Gy | AA depleted Day 5 |
| 12 | Sat | 0 Gy | Habitual |
| 13 | Sun | 0 Gy | Habitual |
| 14 | Mon | 2 Gy | AA depleted Day 1 |
| 15 | Tue | 2 Gy | AA depleted Day 2 |
| 16 | Wed | 2 Gy | AA depleted Day 3 |
| 17 | Thu | 2 Gy | AA depleted Day 4 |
| 18 | Fri | 2 Gy | AA depleted Day 5 |
| 19 | Sat | 0 Gy | Habitual |
| 20 | Sun | 0 Gy | Habitual |
| 21 | Mon | 2 Gy | AA depleted Day 1 |
| 22 | Tue | 2 Gy | AA depleted Day 2 |
| 23 | Wed | 2 Gy | AA depleted Day 3 |
| 24 | Thu | 2 Gy | AA depleted Day 4 |
| 25 | Fri | 2 Gy | AA depleted Day 5 |
| 26 | Sat | 0 Gy | Habitual |
| 27 | Sun | 0 Gy | Habitual |
| 28 | Mon | 2 Gy | AA depleted Day 1 |
| 29 | Tue | 2 Gy | AA depleted Day 2 |
| 30 | Wed | 2 Gy | AA depleted Day 3 |
| 31 | Thu | 2 Gy | AA depleted Day 4 |
| 32 | Fri | 2 Gy | AA depleted Day 5 |
| 33 | Sat | 0 Gy | Habitual |
| 34 | Sun | 0 Gy | Habitual |
| Chemotherapy | Ongoing | n/a | Cycle 5 days of AA |
| | | | depleted diet + 2 days |
| | | | habitual diet |
| | | | throughout |
| | | | Chemotherapy |
| | | | treatment |

### Example 14: IDO1-Driven Tryptophan Metabolism is a Source of One-Carbon Units for Pancreatic Tumor and Stellate Cells

In vitro and in vivo pancreatic cancer models were used to show that IDO1 expression was highly context dependent, influenced by attachment independent growth as well as canonical activator IFNγ. Cancer cells were also shown to release tryptophan-derived formate, which can be taken up and utilized by pancreatic stellate cells to support purine nucleotide synthesis.

The metabolic consequences of IDO1-driven tryptophan metabolism were evaluated in the context of pancreatic ductal adenocarcinoma (PDAC). PDAC tumors are extremely aggressive, with poor clinical outcomes. Characteristically, PDAC tumors exhibit hypovascularization, deranged metabolism, and contain a large proportion of complex stroma. Non-cancerous stromal stellate cells can support tumor cell metabolism through the provision of nutrients such as alanine. Unlike other tumor models, PDAC-bearing mice are unresponsive to serine restriction.

Analysis of public data showed that several tumor types—including pancreatic cancer—had high-IDO1 expressing sub-sets. IDO1 was expressed in genetically engineered mouse models for PDAC. The resulted showed that IDO1 expression was not well represented in standard in vitro cell culture conditions, but could be induced by the canonical activator IFNγ, or by culture in low attachment conditions, which regulate IDO1 via JAK/STAT signaling. The results also showed that when IDO1 was expressed by cancer cells, IDO1 promoted the generation of one-carbon units from tryptophan that are used in de novo purine nucleotide synthesis. Further, tryptophan-derived formate was released by cancer cells. Pancreatic stellate cells (a key component of the tumor stroma) captured the exogenously derived formate and channeled the formate into de novo nucleotide synthesis.

### Experimental Methods

Cell culture: All cell lines used in the study were cultured at 37° C. in 5% CO₂ in a humidified incubator. Cell lines were authenticated using Promega GenePrint 10 and tested for Mycoplasma using Mycoalert (Lonza). AsPC-1 (female), BxPC-3 (female), CFPAC-1 (male), HPAF-II (male), Panc 10.05 (male) & SW 1990 (male) cells were cultured in RPMI supplemented with 10% FBS, 1% penicillin-streptomycin, 0.2% amphotericin B and glutamine (2 mM). Mouse ImPSC and KPC cell lines were cultured in DMEM supplemented with 10% FBS, 1% penicillin-streptomycin, 0.2% amphotericin B and glutamine (2 mM). KPC lines were isolated from the tumors of Pdx1-cre;LSL-Kras^{G12D/+};LSL-Trp53^{R172H/+} mice either with a mixed or pure C57BL/J background. KPC-IDO1 & KPC-EV cell lines were made from pure C57BL/J KPC cells using the PiggyBac transposon system. ImPSC #2 and #3 lines were isolated from Pdgfra^{tm11(EGFP)Sor} mice.

Mice: Mus musculus cohorts were housed in a barrier facility proactive in environmental enrichment and maintained on a normal chow diet. Mixed male and female populations were used for each genotype. Cohorts were on a mixed strain background but all cohorts consisted of litter-matched controls and were killed at a humane clinical end point. For allograft of mixed background KPC cells, Crl:CD1-Foxn1^{nu} (CD1-Nude) female mice were used (7 weeks old). For allograft of pure C57BL/J KPC cells, C57BL/J female mice were used (7 weeks old).

Extraction of ImPSC cell lines: Healthy pancreas tissue extracted from C57BL/J mice was minced and digested for 20 mins at 37° C. with 0.1% DNase, 0.05% Collagenase P and 0.02% Pronase in Gey's balanced salt solution (GBSS). The tissue was then triturated until the large pieces were no longer visible, passed through a 100 μm filter and washed with GBSS. The cells were then pelleted and resuspended in 9.5 mL GBSS with 0.3% BSA and 8 mL Nycodenz solution. The cell suspension was layered beneath GBSS containing 0.3% BSA, and centrifuged at 1400×g for 20 min at 4° C. Stellate cells were harvested from the interface of the Nycodenz solution at the bottom and the aqueous solution at the top. The PSCs isolated were then washed with GBSS and resuspended in DMEM with 10% characterized FBS (HyClone), 100 U/mL penicillin and 100 μg/mL streptomycin. The cells were immortalized with pRetro. Super. shARF retroviral plasmid and selected with blasticidin (404).

ImPSC #2 and #3 lines were isolated using a very similar protocol as ImPSC #1 with some minor differences detailed below. Pancreas tissue was extracted from Pdgfra^{tm11(EGFP)Sor} mice, minced with a scalpel and digested with 0.1% DNase I and 0.05% collagenase P in GBSS for 30 mins at 37° C. The solution was then passed through a 100 μm filter, washed with GBSS, pelleted and resuspended in 6 mL GBSS containing 0.3% BSA. The cell suspension was then mixed with 8 mL Histodenz solution (43.75% in GBSS), layered beneath GBSS containing 0.3% BSA, and centrifuged at 1400×g for 20 mins at 4° C. Stellate cells were harvested from the interface of the Histodenz solution at the bottom and the aqueous solution at the top. The PSCs were washed in PBS containing 3% FBS and resuspended in DMEM containing 10% FBS, 1% penicillin-streptomycin, 0.2% amphotericin B and glutamine (2 mM). After the culture was established, fibroblasts expressing GFP were isolated via FACS and immortalized spontaneously.

ImPSC #1 cells stably expressing GFP (ImPSC-GFP cells) were generated by the PiggyBac transposon system. Briefly, 5×10⁴ ImPSC #1 cells were seeded in a 6-well plate. 24 h after seeding, cells were transfected using Lipofectamine 3000 with 1.5 μg Super piggyBac Transposase expression vector and 0.6 μg PB-GFP PB-CMV-MCS-EF1-GreenPuro. 24 h after transfection, cells were selected in 5 μg/mL puromycin for 48 h, until puromycin sensitive control cells treated in parallel were dead.

Production of KPC-EV and KPC-IDO1 cell lines: Pure C57BL/J KPC cells stably expressing IDO1-RFP or RFP only (empty vector control) were generated using the piggyback system. Human IDO1 cDNA was cloned into the PB-RFP PB-CMV-MCS-EF1-RedPuro cDNA cloning and expression vector using XbaI and EcoRI. Successful cloning was confirmed by full sequencing of the insert. 2.5×10⁵ pure C57BL/J KPC cells were seeded in a 6-well plate. 24 h after seeding cells were transfected using Lipofectamine 3000 with 1.5 μg Super piggyBac Transposase expression vector and 0.6 μg of either PB-RFP PB-CMV-IDO1-EF1-RedPuro (IDO1 overexpression) or PB-RFP PB-CMV-MCS-EF1-RedPuro (empty vector control). 24 h after transfection, cells were selected in 5 μg/mL puromycin for 48 h, until puromycin sensitive control cells treated in parallel were dead. To identify high expressers of IDO1 cells were grown as clones and validated for expression by immunoblotting.

Hypoxia experiments: Cells were seeded and allowed to grow for 48 h to ˜80% confluence under normal tissue culture conditions. Cells were then transferred to a humidified Whitley H35 hypoxystation controlled by a hypoxic gas mixer at 37° C. with 1% O₂, 5% CO₂, and 94% N₂ for 24 h prior to lysis following standard RIPA lysis protocol.

Attachment-independent 3D growth experiments: 1×10⁶ cells were seeded in ultra-low attachment plates for 48 h. For treatments, cells were collected, centrifuged at 50×g for 5 mins and washed in PBS. Cells were then resuspended in 2 mL of treatment medium and transferred back into ultra-low attachment plates for indicated treatment times.

Conditions medium experiments: 8.7×10⁶ HPAF-II or CFPAC-1 cells were seeded in 10 cm dishes in their normal growth media. Experimental media for conditioning were formulated lacking tryptophan and supplemented with the stated concentrations of ¹³C₁₁-tryptophan. After 48 h in culture, cells were washed in PBS and media for conditioning was added. After 48 h, conditioned medium was collected and passed through a 0.45 μm filter to remove cells. Conditioned medium was stored at −20° C. prior to use.

Co-culture experiments: 5×10⁵ ImPSC-GFP cells were seeded either alone or with 1×10⁴ CFPAC-1 cells in 6-well plates. Experimental media was formulated lacking tryptophan and supplemented with 0.4 mM ¹³C₁₁ tryptophan. After 24 h in culture, cells were washed in PBS and media containing human IFNγ (1 ng/mL) or vehicle only control and/or epacadostat (1 μM) or vehicle only control was added. After 24 h, cells were detached by trypsinization, washed in PBS and resuspended to a concentration of 1×10⁷ cells/mL in cold supplemented PBS (PBS+3% FBS, 5 mM glucose, MEM Amino Acids and MEM NEAA). The cell suspensions were then passed through a 70 μm mesh to ensure a single-cell suspension and subjected to fluorescence-activated cell sorting (FACS) using an Aria sorter Z6001 to separate GFP-positive cells from unlabeled cells. The resultant cell suspension was centrifuged at 300×g for 5 mins and the pellet was resuspended in ice-cold lysis solvent. Using the cell counts obtained from FACS, the volume of lysis solvent was normalized to 2×10⁶ cells per ml. Subsequent isolation of metabolites for LCMS was performed as below.

Western blotting: Protein was extracted from whole cells by lysis in RIPA buffer supplemented with protease and phosphate inhibitor cocktail. For cells grown in ultra-low attachment plates, cells were collected by centrifugation at 50×g for 5 mins, washed in PBS and resuspended in 100 μL RIPA lysis buffer. Cells were left to lyse on ice for 10 mins and then homogenized by pipetting. For adherent cells, cells were washed in PBS and lysed in 200 RIPA lysis buffer on ice in situ, collected using a cell scraper and homogenized by pipetting. Tissue samples were snap-frozen and stored at −80° C. Frozen samples were weighed before lysis to ensure a minimum sample size of 20 mg. Samples were homogenized in 2 mL RIPA lysis buffer using a TissueLyser II. Lysates were cleared by centrifugation at 12,000×g for 15 mins at 4° C. Supernatants were collected and total protein content quantified by BCA assay. Lysates were normalized by total protein content and prepared for western blotting with the addition of 4× Bolt™ LDS Sample Buffer (+355 mM β-mercaptoethanol) and heated to 95° C. for 10 mins. Lysates (25 μg) were resolved on Bolt™ 4-12% bis-tris plus pre-cast gels using Bolt™ MOPS SDS Running Buffer running buffer and transferred to nitrocellulose membranes. When total protein staining was performed, it was done prior to blocking using Revert™ Total Protein Stain. Membranes were blocked for 1 hour using Odyssey® Blocking Buffer (TB S) and incubated overnight at 4° C. with primary antibodies. All primary antibodies were diluted in Odyssey® Blocking Buffer at a concentration of 1:1000, except actin, which was used at 1:10,000. Membranes were washed three times in TBS+1% TWEEN® 20 and incubated with secondary antibodies (1:10,000) for 1 h at room temperature. Fluorescence intensity was captured and quantified using a LI-COR Odyssey® Fc Imaging System with Image Studio software (version 5.2).

In vivo models: LSL-Kras^{G12D/+}, Pdx1-cre;LSL-Kras^{G12D/+};Trp53^{fl/+} and Pdx1-cre;LSL-Kras^{G12D/+};LSL-Trp53^{R172H/+} mice were allowed to develop tumors, killed at humane clinical endpoint and tumors removed for analysis. Pancreas tissue from healthy non-cre-expressing littermates were used as controls. For allograft experiments, pure C567Bl6/J KPC cells were implanted by unilateral subcutaneous injections (1×10⁶ cells per injection) into pure C567Bl6/J female mice. Mixed background KPC cells were implanted by unilateral subcutaneous injections (2×10⁶ cells per flank) into Crl:CD1-Foxn1^{nu} (CD1-Nude) female mice. Mice were monitored daily until they reached clinical end point or tumor size reached 300 mm³. Mice were fasted for 3 h and then received an intraperitoneal injection of 800 μL of 120 mM ¹³C Tryptophan. 3 h after injection, mice were killed and tumors removed for analysis.

LCMS for steady state metabolite measurements: Cells were seeded into 6-well plates in complete medium and allowed to grow to ˜80% confluence. Cells were washed with PBS and the relevant experimental media were added for the stated times. Duplicate wells were used for cell counting: cell counts (2D cells) or protein concentration (3D cells BCA assay) were used to normalize the volume of lysis solvent prior to metabolite extractions (1×10⁶ cells per ml). For 2D grown cells, cells were washed quickly in PBS, then ice-cold lysis solvent (Methanol 50%, acetonitrile 30%, water 20%) was added and cells scraped on ice. For 3D grown cells, cells were transferred to 15 mL falcon tubes and centrifuged at 50×g for 5 minutes. The supernatant was removed and the cell pellet was washed in PBS and centrifuged again. The supernatant was removed and the cell pellet resuspended in ice-cold lysis solvent. Lysates were transferred to 1.5 ml tubes on ice, vortexed, then centrifuged at 18,000×g at 4° C. for 10 mins. Supernatants were collected and stored at −80° C. for LCMS analysis. Tissue samples were snap-frozen and stored at −80° C. Frozen samples were weighed before lysis. Samples were homogenized in 2 mL ice cold lysis solvent using a TissueLyser II. Lysates were then cleared of protein by centrifugation at 18,000×g for 10 mins at 4° C. and then normalized to 10 mg/mL with lysis buffer based on original tissue mass.

GCMS for formate analysis: 40 μL of sample was added to 20 μL of d2-formate (50 μM, internal standard), 50 μL pyridine, 10 μL NaOH (1N), and 5 μL benzyl alcohol. While vortexing, 20 μL of methyl chloroformate was added to this mixture for derivatization. 100 μL methyl tertiary butyl ether and 200 μL H₂O were then added, and the samples subsequently vortexed for 10 s and centrifuged for 10 mins at maximum speed. The apolar phase was then transferred to a GC-vial and capped. Standards and blank samples (water) were prepared in the same manner and analyzed with the experimental samples to subtract the background and validate the quantification. MassHunter Quantitative analysis software was used to extract and process the peak areas for formate, 2-formate and ¹³C formate. After correction for background signals, quantification was performed by comparing the peak area of formate (m/z of 136) and 13C formate (m/z of 137) against that of d 2-formate (m/z of 138).

Sample analysis was performed using an LCMS platform consisting of an Accela 600 LC system and an Exactive mass spectrometer. A Sequant ZIC-pHILIC column (4.6 mm×150 mm, 3.5 μm) was used to separate the metabolites with the mobile phase mixed by A=0.1% (v/v) formic acid in water and B=0.1% (v/v) formic acid in acetonitrile. A gradient program starting at 20% of A and linearly increasing to 80% at 30 min was used followed by washing (92% of A for 5 mins) and re-equilibration (20% of A for 10 min) steps. The total run time of the method was 45 min. The LC stream was desolvated and ionized in the HESI probe. The Exactive mass spectrometer was operated in full scan mode over a mass range of 70-1,200 m/z at a resolution of 50,000 with polarity switching. The LCMS raw data was converted into mzML files by using ProteoWizard and imported to MZMine 2.10 for peak extraction and sample alignment. A house-made database including all possible ¹³C and ¹⁵N isotopic m/z values of the relevant metabolites was used for the assignment of LCMS signals. Finally the peak areas were used for comparative quantification.

Carbon-13 labelling of metabolites: Experimental media were formulated lacking tryptophan or serine and supplemental with the stated concentrations of ¹³C₁₁-tryptophan, ¹³C₃¹⁵N₁-serine, or ¹³C₁-formate. The same basic protocol was used as for steady state metabolite measurements. Metabolites were extracted as above.

### Results

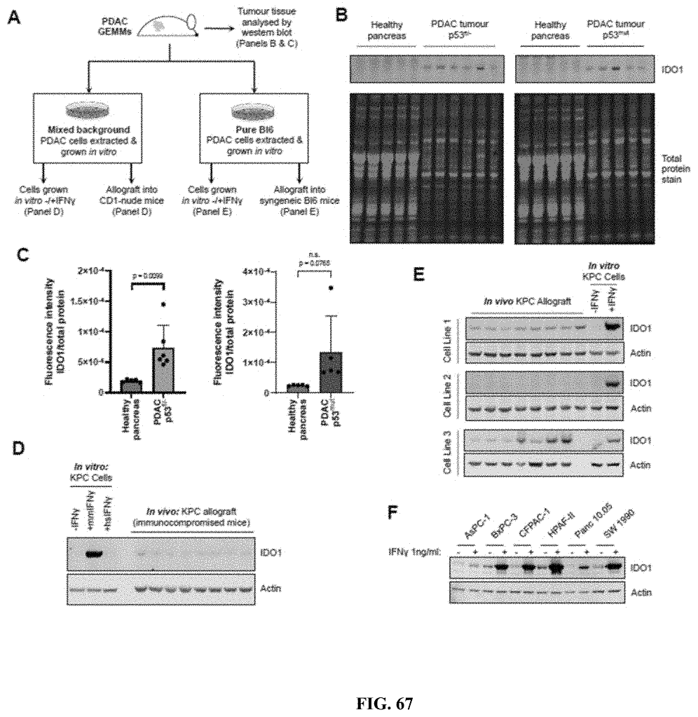

PDAC cells express IDO1 in a context-dependent manner: The expression of IDO1 in pancreatic cancer cells was investigated in vitro and in vivo. Utilizing murine KPC models, IDO1 expression was assessed in a range of contexts (FIG. 67): Direct analysis of pancreatic tumor tissue from Pdx1-cre;LSL-Kras^{G12D/+}; Trp53^{fl/+} and Pdx1-cre;LSL-Kras^{G12D/+};LSL-Trp53^{R172H/+} mice showed that tumors had increased IDO1 expression versus normal pancreas tissue, and that certain tumors expressed high levels of IDO1 (FIG. 67 PANEL B and PANEL C). Compared to the GEMM tumor tissue, tumor derived primary KPC cells cultured under normal in vitro conditions displayed undetectable IDO1 (FIG. 67 PANEL D). Addition of the murine form of cytokine IFNγ—a canonical activator of IDO1—increased IDO1 expression in vitro. The human form of IFNγ did not impact IDO1 expression in murine cells (FIG. 67 PANEL D). To assess whether in vivo growth could restore IDO1 expression, KPC cells were injected into CD-1 nude mice as subcutaneous allografts. Assessment of IDO1 expression in allograft tumor tissue revealed extremely low IDO1 expression (FIG. 67 PANEL D).

Given the ability of IFNγ to promote IDO1 expression, and the known immunological role of IDO1 increased IDO1 expression in an immuno-competent host was tested using KPC cells. Primary tumor cells extracted from Pdx1-cre; LSL-Kras^{G12D/+}; and LSL-Trp53^{R172H/+} mice with pure C57BL/J background were used, which were successfully engrafted into normal recipient C57BL/J mice. When the cells were injected into syngeneic immunocompetent mice as subcutaneous allograft, IDO1 expression was elevated in tumor tissue (versus in vitro culture) in two of three cell lines (FIG. 67 PANEL E).

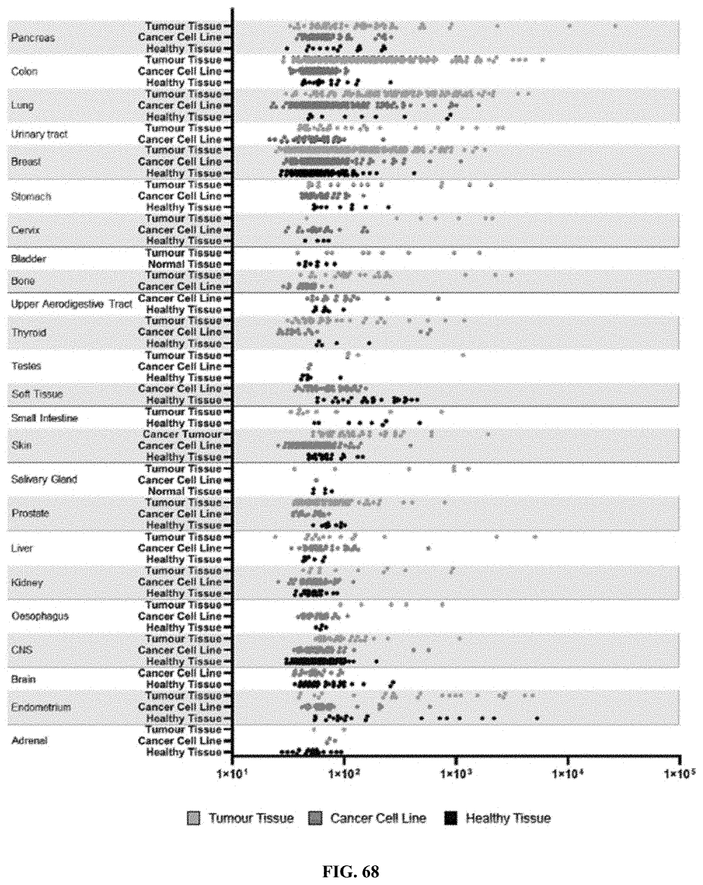

The expression of IDO1 in a panel of human pancreatic cancer cells was also investigated. Similar to KPC cells, IDO1 expression was very low or undetectable under normal culture conditions (FIG. 67 PANEL F). Addition of IFNγ (human form) consistently increased IDO1 expression. To globally assess IDO1 expression in human cancers, data were extracted from the metabolic gene rapid visualizer. In the pancreas, IDO1 had a similar range of expression in healthy tissue compared to cancer cell lines grown in vitro (FIG. 68). However, pancreatic tumor tissue had multiple high or very high IDO1-expressing tumors. The trend was also observed in a variety of other tumors, particularly in the colon, breast, and cervix. The dataset showed consistently that IDO1 expression was elevated in tumor versus healthy tissue, but cancer cells grown under normal in vitro culture conditions did not necessarily display tumor relevant levels of IDO1 Overall, these data showed that IDO1 expression was up-regulated during tumor formation in an immune competent setting.

FIG. 67 shows IDO1 expression in vivo. PANEL A shows a schematic detailing the methods used to analyze IDO1 expression in genetically engineered mouse models (GEMM) of pancreatic ductal adenocarcinoma (PDAC). Tumors from Pdx1-cre;Kras^{G12D/+};Trp53^{fl/+} and Pdx1-cre;Kras^{G12D/+};Trp53^{R172H/+} mice and healthy pancreas tissue from non-cre-expressing isogenic control mice were lysed. PANEL B shows the indicated proteins after analysis by immunoblotting. PANEL C shows the indicated proteins were analyzed using fluorescence intensity of IDO1 relative to total protein (load control) quantified (healthy pancreas n=5, Pdx1-cre;Kras^{G12D/+};Trp53^{fl/+} tumors n=6, Pdx1-cre;Kras^{G12D/+};Trp53^{R172H/+} tumors n=5, unpaired t-tests, p values shown, error bars are std. dev.). PANEL D shows KPC A cells, a line isolated from tumors of mixed-background Pdx1-cre;Kras^{G12D/+};Trp53^{R172H/+} mice were either treated with mouse IFNγ (1 ng/ml) for 24 h, or subcutaneously injected into the flank of CD1-nude mice to form tumors. Cell and tumor lysates were subjected to immunoblotting for the indicated proteins. PANEL E shows KPC cells were isolated from pure C567Bl6/J background Pdx1-cre;Kras^{G12D/+};Trp53^{R172H/+} mice and either treated in culture with mouse IFNγ (1 ng/ml) for 24 h or subcutaneously injected into the flank of C567Bl6/J mice to form tumors. Cell and tumor lysates were subjected to immunoblotting for the indicated proteins. PANEL F shows the indicated cell lines were treated with human IFNγ (1 ng/mL) for 24 h and cell lysates blotted for the indicated proteins.

FIG. 68 shows data extracted from the MERAV database showing the relative abundance of IDO1 mRNA from microarrays.

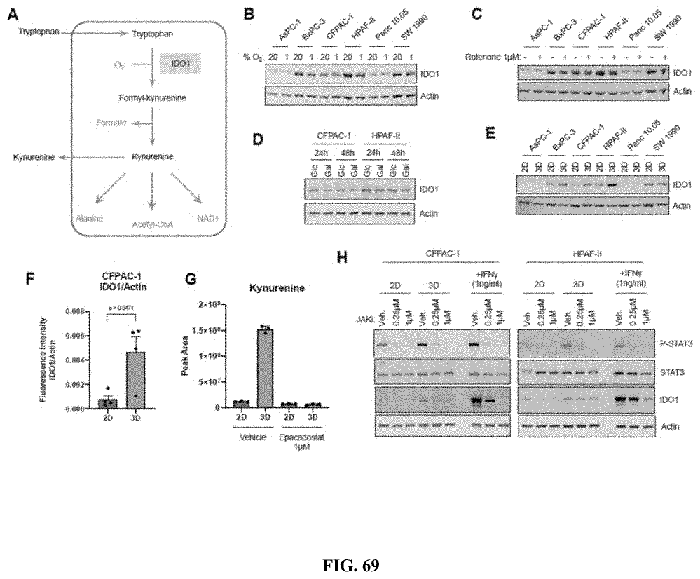

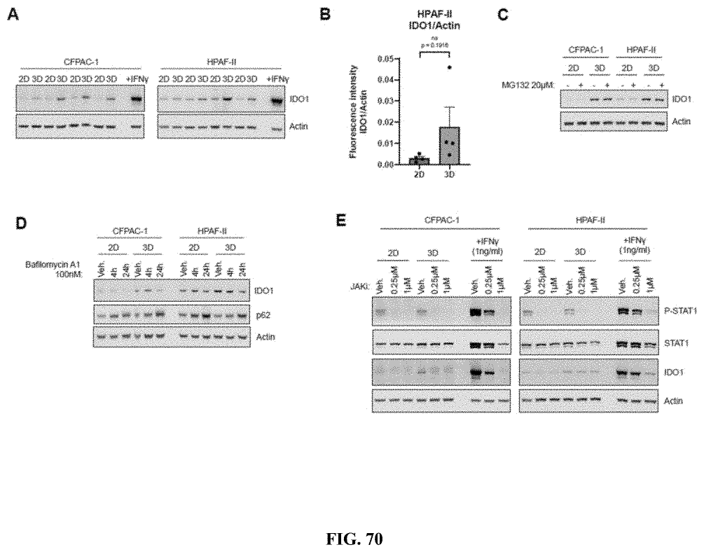

IDO1 expression can be regulated by attachment independent growth in vitro: IDO1 expression promotes the utilization of tryptophan via the kynurenine pathway. Given the diversity of potential metabolic interactions of this pathway (FIG. 69 PANEL A) the effect of immune independent stimuli on IDO1 expression was investigated. Mitochondrial metabolism is potentially linked to the kynureneine pathway in two ways: (1) mitochondrial production of superoxide and (2) entry of tryptophan derived carbons into the TCA cycle via α-ketoadipate. Exposure of PDAC cells to low oxygen or rotenone—both predicated to impact OXPHOS and potentially modulate superoxide levels—had little impact on IDO1 expression (FIG. 69 PANEL B and PANEL C). Similarly, substitution of glucose with galactose (to promote OXPHOS) did not modulate IDO1 expression (FIG. 69 PANEL D). Transferring cells from 2D monolayer culture to attachment-independent growth (without any other adjustments to culture conditions) caused increased IDO1 expression in BxPC-3, CFPAC-1, and HPAF-II cells (FIG. 69 PANEL E, PANEL F, FIG. 70 PANEL A and PANEL B). The observations were consistent in CFPAC-1 cells and were accompanied by a dramatic increase in kynurenine pathway activity, as measured by kynurenine efflux, which was ablated by IDO1 inhibitor epacadostat (FIG. 69 PANEL G).

FIG. 69 shows that IDO expression was upregulated by 3D growth and IFNγ via JAK/STAT signaling. PANEL A shows a schematic detailing the kynurenine pathway through which tryptophan is metabolized. The indicated proteins were analyzed by immunoblotting in the indicated cell lines after 24 h of culture. PANEL B shows proteins cultured under either normoxic (20% 02) or hypoxic (1% 02) conditions; PANEL C shows proteins treated with rotenone (1 μM) or vehicle only control; and PANEL D shows proteins cultured in media containing either glucose (Glc) (10 mM) or galactose (Gal) (10 mM). The indicated cell lines were cultured in 2D or 3D conditions for 24 h, and cell lysates were immunoblotted for the indicated proteins. PANEL E shows proteins cultured in 2D or 3D conditions. PANEL F shows the fluorescence intensities of IDO1/Actin for CFPAC-1 in 2D and 3D conditions, quantified (n=4, paired t-test, p value shown, error bars are S.E.M.). PANEL G shows the results of CFPAC-1 cells cultured in 2D or 3D conditions for 24 h and treated with epacadostat (1 μM) or vehicle only control for 16 h before media kynurenine was analyzed by LCMS (1 ex, triplicate wells, error bars are std. dev.). PANEL H shows CFPAC-1 or HPAF-II cells cultured in either 2D or 3D conditions for 24 h and then treated for 16 h with JAKi (1 μM) or vehicle only control (veh.) and/or human IFNγ (1 ng/ml). Cells were then lysed and indicated proteins analyzed by immunoblotting.

In FIG. 70, CFPAC-1 or HPAF-II cells were either growth in normal tissue culture plates (2D) or in ultra-low-attachment tissue culture plates (3D) for 24 h, or cultured in 2D and treated with 1 ng/ml IFNγ. Lysates were (PANEL A) blotted for the indicated proteins and (PANEL B) fluorescence intensity of IDO1 relative to actin (load control) quantified (n=4, paired t-test, p value shown, error bars are S.E.M.). Indicated cell lines were grown in either 2D or 3D for 24 hours and lysates immunoblotted for indicated proteins (PANEL C) after 16 h treatment with MG132 (20 μM) or vehicle-only control (PANEL D) after treatment for the indicated times with bafilomycin A1 (100 nM) or vehicle-only control or (PANEL E) after 16 h treatment with JAKi (at indicated concentrations), vehicle-only control or IFNγ (1 ng/ml).

Attachment independent (AI) growth stimulated IDO1 expression is regulated through JAK/STAT signaling: The molecular mechanism through which attachment independent (AI) growth up-regulates IDO1 expression was studied. Treatment with the proteasome inhibitor MG132 (FIG. 70 PANEL C) or with lysosomal inhibitor bafilomycin (FIG. 70 PANEL D) had no effect on IDO1 protein levels. The data showed that increased IDO1 levels observed during AI growth were not due to changes in IDO1 degradation via proteasomal or lysosomal systems.

IFNγ mediates changes in gene expression through activation of the JAK/STAT signaling cascade. The role of AI growth, independent of IFNγ, on activation of the JAK/STAT pathway leading to increased IDO1 expression was investigated. Activation of STAT proteins (by phosphorylation) was studied using a small molecule JAK inhibitor I (JAKi). The results showed that STAT3 phosphorylation was increased upon AI growth (FIG. 69 PANEL H), indicating up-regulated JAK/STAT pathway activation. This appeared to be specific to STAT3, as no such increase was detected for STAT1 (FIG. 70 PANEL E). Up-regulation of IDO1 protein levels in AI-grown cells was blocked by treatment with JAKi (FIG. 69 PANEL H). These data indicated that during AI growth, the JAK/STAT pathway was activated (similar to IFNγ treatment), and that the stimulates increased IDO1 expression.

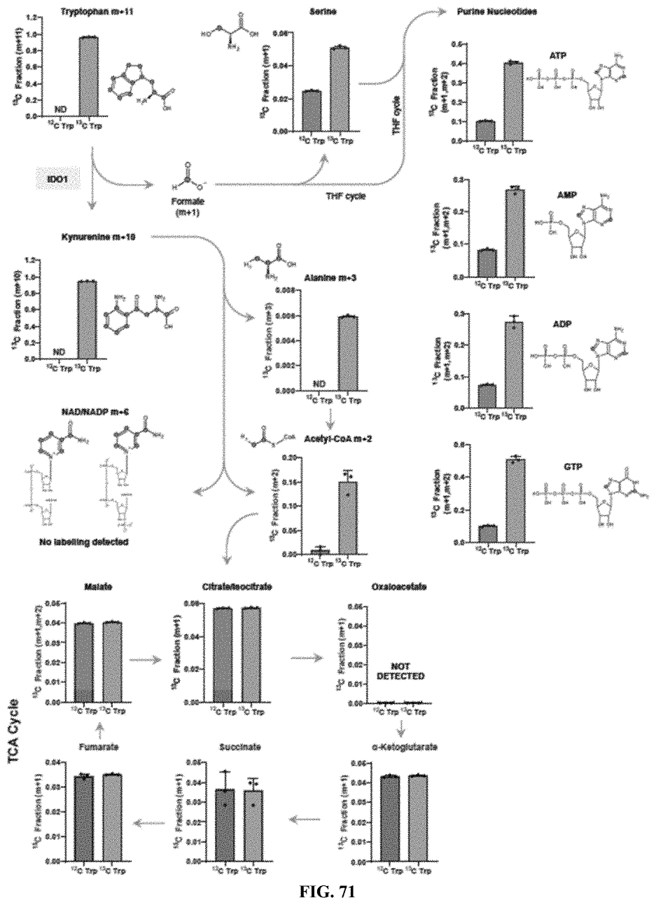

Tryptophan contributes one-carbon units to purine synthesis in vitro: During the metabolism of tryptophan through the kynurenine pathway, a number of metabolites are formed that are known to have potentially important roles in cancer metabolism (FIG. 69 PANEL A). To investigate the production of such metabolites from tryptophan in cancer cells expressing IDO1, cells were cultured with IFNγ in the presence of ¹³C₁₁-tryptophan. Liquid chromatography mass spectrometry (LCMS) was used to track the incorporation of labelled carbons into kynurenine pathway metabolites, and beyond into nucleotide synthesis and the TCA cycle. Cells readily took up ¹³C₁₁-tryptophan, and the cellular tryptophan pool was fully labelled over 24 h. A high fraction (˜95%) of labelling in kynurenine was observed (FIG. 71). Downstream of kynurenine, a small amount of labelling was identified in alanine, however, this was a very small proportion of the total alanine pool (<1%). Tryptophan can by metabolized to acetyl-coA either via alanine or via α-ketoadipate. Some evidence of labelling from tryptophan in acetyl-coA was also detected (FIG. 71). No evidence of labelling in components of the TCA cycle or in NAD/H or NADP/H were observed, suggesting a limited impact of tryptophan on these pathways within PDAC cells.

During the production of kynurenine from tryptophan, a one-carbon unit is released as formate. A potential destination for this formate is to enter the THF cycle. From here, the tryptophan-derived carbon could be used in a number of anabolic pathways, including purine nucleotide synthesis. Tryptophan-derived carbons were observed in purine nucleotides (FIG. 71), indicating that tryptophan is a legitimate source of one-carbon units for the THF cycle in PDAC cells. Another THF-dependent fate for one-carbons is de novo serine synthesis (by combination with glycine via SHMT1/2), and increased labelling of serine from labelled tryptophan was also observed.

The extent of labelling seen in purines was notable, especially given these cells were grown with ample exogenous serine—a major one-carbon source (medium contained 0.4 mM serine compared to 0.08 mM tryptophan). Approximately 30% of the ATP pool and 40% of the GTP pool was labelled by tryptophan-derived carbon. Labelling in serine was a much lower fraction (approximately 3%), suggesting that purine labelling occurs directly via the THF cycle.

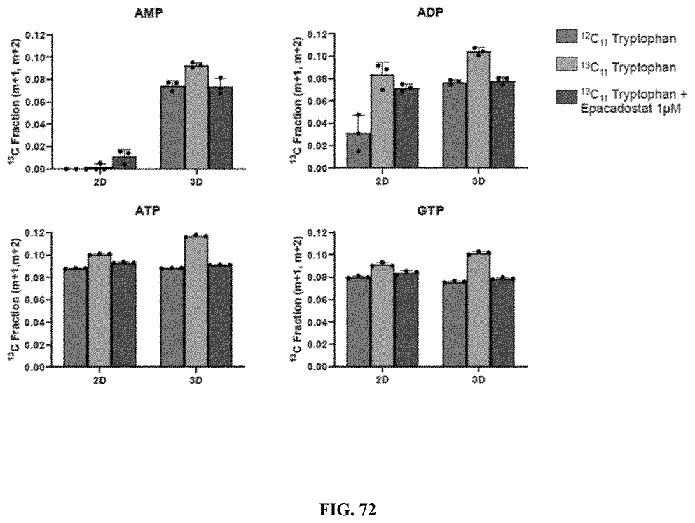

AI growth was confirmed to stimulate the incorporation of tryptophan-derived one-carbon units into purine nucleotides, finding an increased fraction of labelling in AMP, ADP, ATP and GTP within AI-grown cells (FIG. 72), albeit to a lesser extent than seen in IFNγ treated cells. Labelling was prevented by treatment with IDO1 inhibitor epacadostat (FIG. 72). Overall these data clearly indicate that it is possible for IDO1-expressing PDAC cells to utilize tryptophan as a significant source of one-carbon units for purine nucleotide synthesis.

FIG. 72 shows CFPAC-1 cells cultured in 2D or 3D for 24 h, then treated for 24 h with epacadostat (1 μM) or vehicle only control in the presence of either unlabeled (¹²C) or ¹³C₁₁ tryptophan and intracellular quantities of the indicated nucleotides were analyzed by LCMS (1 ex, triplicate wells, error bars are std. dev.).

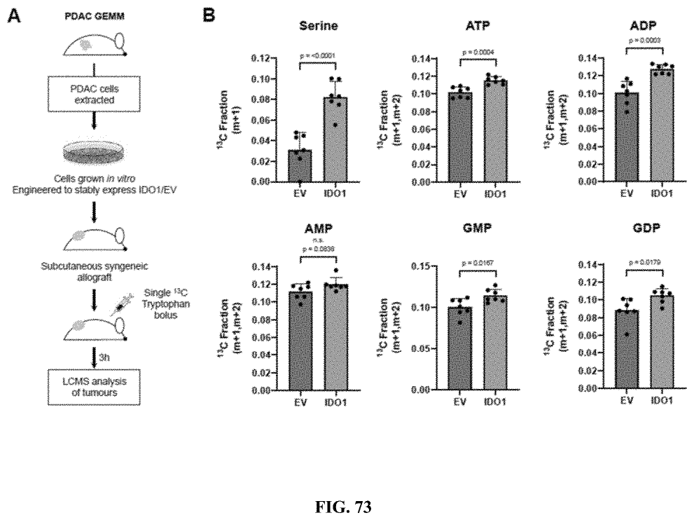

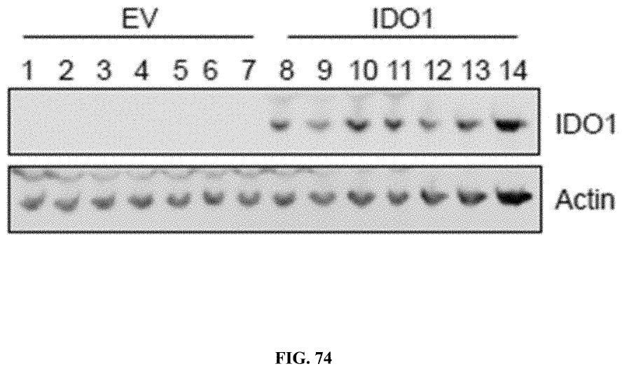

Tryptophan can contribute one-carbon units to purine synthesis in vivo: Whether tryptophan can contribute one-carbon units to purine synthesis in PDAC tumors in vivo was investigated. Given the high variability in IDO1 expression in autochthonous GEMM and KPC allograft tumors, and in order to faithfully recapitulate the setting of a high-IDO1-expressing tumor, KPC cells were engineered to constitutively express IDO1. The cells were implanted into syngeneic immunocompetent mice as subcutaneous allografts (FIG. 73 PANEL A). Once tumors had formed, mice received a single intraperitoneal injection of ¹³C₁₁-tryptophan solution and we assessed the incorporation of tryptophan-derived carbons using LCMS at a single time-point post injection. We found a significant increase in the labelled fraction in serine, ATP, ADP, GMP and GDP in tumor tissue of IDO1-expressing versus empty vector (EV) controls (FIG. 73 PANEL B, FIG. 74). These results indicate that IDO1-expressing tumors can indeed incorporate tryptophan-derived carbons into purine nucleotides in vivo.

FIG. 73 shows that tryptophan-derived one-carbon units are incorporated into nucleotides in in vivo pancreatic tumors. PANEL A shows a schematic detailing the experimental approaches for this figure. In PANEL B, KPC cells from pure C57BL/J Pdx1-cre;Kras^{G12D/+};Trp53^{R172H/+} mice expressing IDO1 or empty-vector control (EV) were injected subcutaneously into the flanks of C57BL/J mice, once tumors had formed the mice were given 8004, of 120 mM ¹³C₁₁ tryptophan by intraperitoneal injection and left for 3 h. Tumor tissue was excised and analyzed by LCMS (fraction of major isotopologues relative to total are shown, EV n=7, IDO1 n=7, unpaired t-tests, p values are shown, error bars are std. dev.).

FIG. 74 shows data from KPC cells from pure C57BL/J Pdx1-cre;Kras^{G12D/+};Trp53R^{172H/+} mice expressing IDO1 or empty-vector control (EV) were injected subcutaneously into the flanks of C57BL/J mice, once tumors had formed the mice were given 8004, of 120 mM ¹³C₁₁ tryptophan by intraperitoneal injection and left for 3 h. Tumor tissue was excised and analyzed by immunoblotting for the indicated proteins.

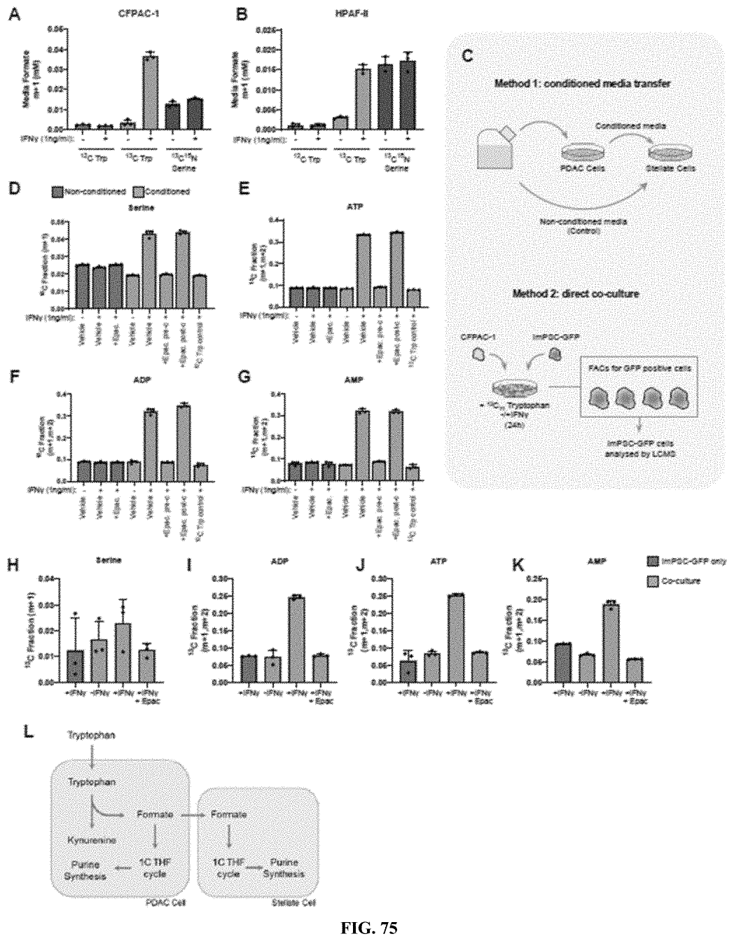

PDAC cells excrete tryptophan-derived formate: Whether IDO1-expressing PDAC cells released formate produced from tryptophan was investigated. After culture with ¹³C₁₁-tryptophan, labelled formate was identified by gas chromatography mass spectrometry in the spent medium from CFPAC-1 and HPAF-II cells expressing IDO1 (+IFNγ) (FIG. 75 PANEL A and PANEL B). The release of tryptophan-derived formate was considerably higher than serine-derived formate in CFPAC-1 cells and equivalent in HPAF-II cells. These results were surprising because serine is generally viewed as the dominant one-carbon source in cancer cells, and because exogenous serine levels are higher than tryptophan.

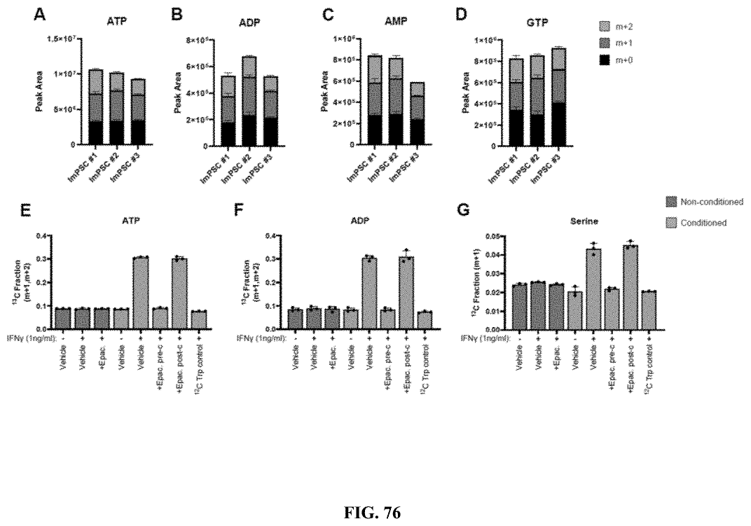

Stellate cells take up tryptophan-derived formate and utilize formate for purine synthesis: The ability of pancreatic stellate cells to take up tryptophan-derived formate released by PDAC cells and utilize it in synthesis of purine nucleotides was investigated. To directly test the ability of pancreatic stellate cells take up and utilize exogenous formate, immortalized mouse stellate cells (ImPSCs) were cultured in media supplemented with ¹³C₁-formate. LCMS analysis revealed that stellate cells consumed extracellular formate and incorporated single carbon into purines. Purine synthesis utilized two THF-derived one carbons, giving rise to major isotopologue peaks of m+1 and m+2 (FIG. 76 PANEL B).

To assess whether tryptophan-derived formate produced within PDAC cells could be used in the same way, two techniques were used: (1) conditioned media transfer, and (2) direct co-culture (FIG. 75 PANEL C). To condition medium, PDAC cells were grown in medium containing ¹³C₁₁-tryptophan and conditioned medium was collected after 24 hours. Filtered medium was then transferred onto stellate cells before analysis of the stellate cells by LCMS. Labelling of purine nucleotides and serine was detected in stellate cells given conditioned medium from IDO1 expressing (+IFNγ) PDAC cells (FIG. 75 PANEL D-PANEL G). Labelling was prevented when the PDAC cells were treated with epacadostat during the conditioning process. Labelling was unaffected when epacadostat was added after conditioning (i.e. while stellate cells were grown with conditioned medium), indicating that IDO1 activity in the PDAC cells, not the stellate cells, was critical for stellate cell formate utilization. The same observations were made when the experiment was repeated with ImPSC #2 cells (FIG. 76 PANEL E-PANEL G).

To further confirm the findings, direct co-culture assays were performed. CFPAC-1 cells were co-cultured for 24 hours with ImPSC cells engineered to ectopically express GFP. The co-culture medium contained ¹³C₁₁-tryptophan and IFNγ. The ImPSC-GFP cells were then separated from the PDAC cells by FACS and subjected to LCMS analysis. Labelling of purine nucleotides was evident in stellate cells co-cultured with PDAC cells in the presence of IFNγ (FIG. 75 PANEL H-PANEL K). With this method, the labelled fractions were generally smaller and clear labelling in serine was not observed. Nucleotide labelling was not seen in stellate cells cultured alone. Importantly, labelling was also lower when IDO1 levels were low (−IFNγ) or IDO1 was inhibited by treatment with epacadostat (FIG. 75 PANEL H-PANEL K).

FIG. 75 shows that cancer cells released tryptophan-derived formate, which was consumed by pancreatic stellate cells and incorporated into nucleotides. CFPAC-1 (PANEL A) or HPAF-II (PANEL B) cells were cultured in 3D for 4 days and then treated with IFNγ (1 ng/ml) or vehicle only control in the presence of either unlabeled (¹²C), ¹³C₁₁ tryptophan, or ¹³C₃¹⁵N₁ serine for 24 h. Media quantities of formate were analyzed by derivatization and GC-MS (1 ex, triplicate wells, error bars are std. dev.). PANEL C shows a schematic of the experimental approaches used in PANEL D-PANEL K. CFPAC-1 cells were treated with vehicle only control or human IFNγ (1 ng/ml) and epacadostat (epac., 1 μM) or vehicle only control in the presence of unlabeled (¹²C) or ¹³C₁₁ tryptophan. Conditioned media was collected after 24 h and ImPSC's were cultured in this media, or in non-conditioned treatment-matched media. After 24 h, intracellular quantities of serine (PANEL D), ATP (PANEL E), ADP (PANEL F) and AMP (PANEL G) were analyzed by LCMS (fraction of major isotopologues relative to total are shown, 1 ex, triplicate wells, error bars are std. dev.). ImPSC-GFP cells were cultured for 24 h in 2D as a monoculture or in co-culture with CFPAC-1 cells. Cells were then treated with vehicle only control or human IFNγ (1 ng/ml) and epacadostat (1 μM) or vehicle only control in the presence of ¹³C₁₁ tryptophan for 24 h. Cells were then trypsinised and sorted using FACS for GFP-positive cells and intracellular quantities of serine (PANEL H), ATP (PANEL I), ADP (PANEL J) and AMP (PANEL K) were analyzed by LCMS (fraction of major isotopologues relative to total are shown, 1 ex, triplicate wells, error bars are std. dev.). PANEL L shows a proposed model for the use of tryptophan-derived formate in pancreatic ductal adenocarcinoma (PDAC) cells and pancreatic stellate cells.

FIG. 76 shows intracellular uptake of ¹³C₁ formate in ATP, DP, AMP, and GTP in ImPSC #1, ImPSC #2, and ImPSC #3 cells. ImPSC #1, ImPSC #2 & ImPSC #3 cells were cultured for 24 h in the presence of ¹³C₁ formate and intracellular quantities of ATP (PANEL A), ADP (PANEL B), AMP (PANEL C) and GTP (PANEL D), all possible destination for formate-derived one carbons were analyzed by LCMS (1 ex, triplicate wells, error bars are std. dev.). CFPAC-1 cells were treated with IFNγ (1 ng/ml) and/or epacadostat (1 μM) and/or vehicle only controls in the presence of unlabeled (¹²C) or ¹³C₁₁ tryptophan. Conditioned media was collected after 24 h and ImPSC #2 cells were cultured in this media, or in non-conditioned treatment-matched media. After 24 h, intracellular quantities of ATP (PANEL E), ADP (PANEL F) and serine (PANEL G) were analyzed by LCMS (fraction of major isotopologues relative to total are shown 1 ex, triplicate wells, error bars are std. dev.).

Concentrating on pancreatic cancer, the relative expression of IDO1 in GEM models was determined. Tumors from KPC mice showed clear elevation of IDO1 compared to healthy tissue in vivo, but an absence of IDO1 expression in cell culture. Expression data from a large set of human cancers further illustrated that high IDO1 expression was seen in multiple tumors but not observed in cell culture.

Beyond the canonical IDO1 activator IFNγ, the impact of metabolic perturbations caused by hypoxia, rotenone treatment or galactose on IDO1 expression was examined. None of the conditions changed IDO1 levels. A transfer of PDAC cells from standard monolayer culture to attachment independent conditions up-regulated IDO1 was observed, albeit to a lesser extent than IFNγ. The mechanism of the effect was investigated, and attachment independent growth regulated IDO1 via the JAK/STAT signaling pathway was observed.

Improved understanding of IDO1 expression allowed a detailed analysis of IDO1-dependent tryptophan metabolism in PDAC cells. The kynurenine pathway made a significant contribution to nucleotide synthesis in PDAC cells in vitro, contributing carbons to approximately 30% of the purine pool over 24 hours. IDO1-dependent tryptophan labelling was detected in tumor serine and purine pools following a single injection of 13C11-tryptophan. PDAC cells were tested for tryptophan-derived formate efflux. Certain PDAC cells released double the quantity of tryptophan-derived versus serine-derived formate, despite exogenous serine outweighing tryptophan 4:1. A robust ability of stellate cells to capture tryptophan-derived formate produced by PDAC cells and incorporate the formate into nucleotide synthesis was also observed.

Overall, the results show that IDO1 can influence one-carbon metabolism in cancer and stromal cells. In IDO1 expressing tumors, tryptophan was shown to be a legitimate one-carbon source for the THF cycle. The data also provide a mechanistic explanation as to why tryptophan can be one of the most highly depleted interstitial nutrients in PDAC.

### Example 15: Effect of Epacadostat on Cell Proliferation and Nucleotide Synthesis

Epacadostat enhances the antiproliferative effect or serine starvation. KPC cells (tumor cells derived from a pancreatic tumor of a Kras' p53″^{t} genetically engineered mouse) were seeded into 24-well plates and allowed to adhere overnight. Cells were washed with PBS and received either control medium containing all amino acids or matched medium lacking serine (−Serine) with or without IDO1 inhibitor epacadostat (1 μM). Cell number was recorded every 24 h for 5 days. A time zero plate was used to calculate the starting cell number.

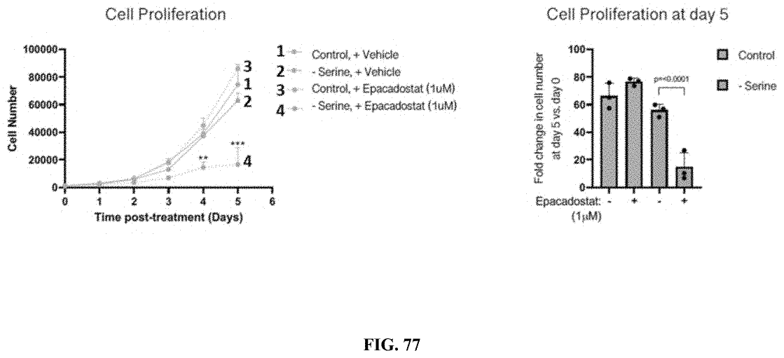

FIG. 77 LEFT PANEL shows cell proliferation over 5 days in cells treated with: 1) control+vehicle; 2) −Serine+vehicle; 3) control+epacadostat (1 μM); or 4) −Serine+epacadostat (1 μM). RIGHT PANEL shows fold changes in cell number at day 5 compared to day 0 in cells treated with: 1) control+vehicle; 2) −Serine+vehicle; 3) control+epacadostat (1 μM); or 4) −Serine+epacadostat (1 μM).

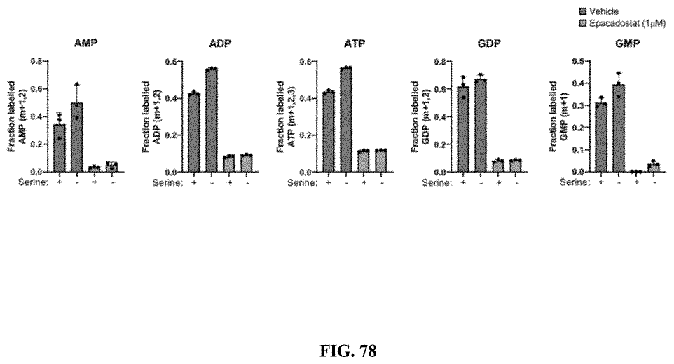

Serine starvation was shown to increase the amount of tryptophan-derived carbon used in nucleotide synthesis in an IDO1-dependent matter. KPC cells (tumor cells derived from a pancreatic tumor of a Kras^{mut} p53^{mut} genetically engineered mouse) were seeded into 6-well plates and adhered overnight. Cells were fed medium containing carbon-13 labelled tryptophan either with (+) or without (−) serine (plus all other amino acids), with or without the IDO1 inhibitor epacadostat (1 μM). After 48 h, metabolites were extracted from cells and analyzed by LCMS. The labelled fraction (derived from carbon-13) of purine nucleotides (ATP, ADP, AMP, GDP, GTP) are shown. FIG. 78 shows the labelled fractions derived from carbon-13 in cells of AMP, ADP, ATP, GDP, and GMP in cells treated with: 1) control+vehicle; 2) −Serine+vehicle; 3) control+epacadostat (1 μM); or 4) −Serine+epacadostat (1 μM).

### Embodiments

The following non-limiting embodiments provide illustrative examples of the invention, but do not limit the scope of the invention.

Embodiment 1. A method of treating a cancer in a subject in need thereof, the method comprising: a) administering to the subject a therapeutically-effective amount of a pharmaceutical composition, wherein the pharmaceutical composition is substantially devoid of at least two amino acids, for a first amount of time; b) a radiation therapy for a second amount of time; and c) after the first amount of time and the second amount of time, waiting a third amount of time, wherein the subject is not administered the pharmaceutical composition or the radiotherapy during the third amount of time.

Embodiment 2. The method of embodiment 1, wherein the cancer is rectal cancer.

Embodiment 3. The method of embodiment 1, wherein the cancer is breast cancer.

Embodiment 4. The method of any one of embodiments 1-3, wherein the administration is oral.

Embodiment 5. The method of any one of embodiments 1-4, wherein the radiation therapy is an external beam therapy.

Embodiment 6. The method of embodiment 5, wherein the external beam therapy is three dimensional conformal radiation therapy (3D-CRT).

Embodiment 7. The method of embodiment 5, wherein the external beam therapy is intensity-modulated radiation therapy (IMRT).

Embodiment 8. The method of any one of embodiments 1-7, wherein the radiation therapy comprises administering about 5 Grays (Gy) to about 50 Gy of radiation to the subject.

Embodiment 9. The method of any one of embodiments 1-8, wherein the radiation therapy comprises administering about 5 Gy of radiation to the subject.

Embodiment 10. The method of any one of embodiments 1-8, wherein the radiation therapy comprises administering about 50 Gy of radiation to the subject.

Embodiment 11. The method of any one of embodiments 1-4 or 8-10, wherein the radiation therapy is an internal beam therapy.

Embodiment 12. The method of any one of embodiments 1-11, wherein the at least two amino acids is serine and glycine.

Embodiment 13. The method of any one of embodiments 1-12, wherein the pharmaceutical composition is further substantially devoid of proline.

Embodiment 14. The method of any one of embodiments 1-13, wherein the pharmaceutical composition is further substantially devoid of cysteine.

Embodiment 15. The method of any one of embodiments 1-14, further comprising administering a high fat diet to the subject.

Embodiment 16. The method of embodiment 15, wherein the high fat diet has greater than about 50% of daily calories from fat.

Embodiment 17. The method of any one of embodiments 1-16, further comprising administering a low carbohydrate diet to the subject.

Embodiment 18. The method of embodiment 17, wherein the low carbohydrate diet has less than about 50% of daily calories from carbohydrates.

Embodiment 19. The method of any one of embodiments 1-18, further comprising administering a low protein diet to the subject.

Embodiment 20. The method of embodiment 19, wherein the low protein diet has less than about 15% of daily calories from whole protein.

Embodiment 21. The method of any one of embodiments 1-20, wherein the first amount of time and the second amount of time are equal.

Embodiment 22. The method of any one of embodiments 1-22, wherein the first amount of time and the second amount of time are 5 days.

Embodiment 23. The method of any one of embodiments 1-22, wherein the first amount of time and the second amount of time is greater than the third amount of time.

Embodiment 24. The method of any one of embodiments 1-23, wherein the third amount of time is 2 days.

Embodiment 25. The method of any one of embodiments 1-24, further comprising repeating steps a), b), and c).

Embodiment 26. A method of treating a cancer in a subject in need thereof, the method comprising: a) administering to the subject a therapeutically-effective amount of a pharmaceutical composition, wherein the pharmaceutical composition is substantially devoid of at least two amino acids; and b) administering a therapeutically effective amount of an immunotherapy, wherein the immunotherapy is administered at least twice per day.

Embodiment 27. The method of embodiment 26, wherein the cancer is pancreatic cancer.

Embodiment 28. The method of embodiment 26, wherein the cancer is colon cancer.

Embodiment 29. The method of embodiment 26, wherein the cancer is breast cancer.

Embodiment 30. The method of embodiment 26, wherein the cancer is cervical cancer.

Embodiment 31. The method of embodiment 26, wherein the cancer is lung cancer.

Embodiment 32. The method of any one of embodiments 26-31, wherein the immunotherapy is an IDO1 inhibitor.

Embodiment 33. The method of embodiment 33, wherein the IDO1 inhibitor is indoximod.

Embodiment 34. The method of embodiment 33, wherein the IDO1 inhibitor is navoximod.

Embodiment 35. The method of embodiment 33, wherein the IDO1 inhibitor is epacadostat.

Embodiment 36. The method of any one of embodiments 26-35, wherein the at least two amino acids is serine and glycine.

Embodiment 37. The method of any one of embodiments 26-36, wherein the pharmaceutical composition is substantially devoid of three amino acids.

Embodiment 38. The method of embodiment 37, wherein the three amino acids are serine, glycine, and proline.

Embodiment 39. The method of embodiment 37, wherein the three amino acids are serine, glycine, and cysteine.

Embodiment 40. The method of any one of embodiments 26-39, wherein the therapeutically effective amount of the immunotherapy is about 25 mg to about 500 mg.

Embodiment 41. The method of any one of embodiments 26-40, wherein the therapeutically effective amount of the immunotherapy is about 25 mg.

Embodiment 42. The method of any one of embodiments 26-40, wherein the therapeutically effective amount of the immunotherapy is about 50 mg.

Embodiment 43. The method of any one of embodiments 26-40, wherein the therapeutically effective amount of the immunotherapy is about 100 mg.

Embodiment 44. The method of any one of embodiments 26-40, wherein the therapeutically effective amount of the immunotherapy is about 300 mg.

Embodiment 45. The method of any one of embodiments 26-44, wherein the immunotherapy is administered twice per day.

Embodiment 46. The method of any one of embodiments 26-44, wherein the immunotherapy is administered three times per day.

Embodiment 47. A method of treating a cancer in a subject in need thereof, the method comprising: a) administering to the subject a therapeutically-effective amount of a pharmaceutical composition, wherein the pharmaceutical composition is substantially devoid of at least two amino acids; and b) a therapeutically-effective amount of epacadostat.

Embodiment 48. The method of embodiment 47, wherein the cancer is pancreatic cancer.

Embodiment 49. The method of embodiment 47, wherein the cancer is colon cancer.

Embodiment 50. The method of embodiment 47, wherein the cancer is breast cancer.

Embodiment 51. The method of embodiment 47, wherein the cancer is cervical cancer.

Embodiment 52. The method of embodiment 47, wherein the cancer is lung cancer.

Embodiment 53. The method of any one of embodiments 47-52, wherein the at least two amino acids is serine and glycine.

Embodiment 54. The method of any one of embodiments 47-53, wherein the pharmaceutical composition is substantially devoid of three amino acids.

Embodiment 55. The method of embodiment 54, wherein the three amino acids are serine, glycine, and proline.

Embodiment 56. The method of embodiment 54, wherein the three amino acids are serine, glycine, and cysteine.

Embodiment 57. The method of any one of embodiments 47-56, wherein the therapeutically effective amount of epacadostat is about 25 mg to about 500 mg.

Embodiment 58. The method of any one of embodiments 47-57, wherein the therapeutically effective amount of epacadostat is about 25 mg.

Embodiment 59. The method of any one of embodiments 47-57, wherein the therapeutically effective amount of epacadostat is about 50 mg.

Embodiment 60. The method of any one of embodiments 47-57, wherein the therapeutically effective amount of epacadostat is about 100 mg.

Embodiment 61. The method of any one of embodiments 47-57, wherein the therapeutically effective amount of epacadostat is about 300 mg.

1-21. (canceled)

22. A method of treating a cancer in a subject in need thereof, the method comprising: a) administering to the subject a therapeutically-effective amount of a pharmaceutical composition for a first amount of time, wherein the pharmaceutical composition is substantially devoid of at least two amino acids; b) administering to the subject a radiation therapy for a second amount of time; and c) after the first amount of time and the second amount of time, waiting a third amount of time, wherein the subject is not administered the pharmaceutical composition or the radiotherapy during the third amount of time.

23. The method of claim 22, wherein the cancer is rectal cancer.

24. The method of claim 22, wherein the cancer is breast cancer.

25. The method of claim 22, wherein the administering of the pharmaceutical composition is oral.

26. The method of claim 22, wherein the radiation therapy is an external beam therapy.

27. The method of claim 26, wherein the external beam therapy is three dimensional conformal radiation therapy (3D-CRT).

28. The method of claim 26, wherein the external beam therapy is intensity-modulated radiation therapy (IMRT).

29. The method of claim 22, wherein the radiation therapy comprises administering about 5 Grays (Gy) to about 50 Gy of radiation to the subject.

30. The method of claim 22, wherein the radiation therapy is an internal beam therapy.

31. The method of claim 22, wherein the at least two amino acids is serine and glycine.

32. The method of claim 31, wherein the pharmaceutical composition is further substantially devoid of proline.

33. The method of claim 22, further comprising administering a high fat diet to the subject, wherein the high fat diet has greater than about 50% of daily calories from fat.

34. The method of claim 22, further comprising administering a low carbohydrate diet to the subject, wherein the low carbohydrate diet has less than about 50% of daily calories from carbohydrates.

35. The method of claim 22, further comprising administering a low carbohydrate diet to the subject, wherein the low carbohydrate diet has less than about 10% of daily calories from carbohydrates.

36. The method of claim 22, further comprising administering a low protein diet to the subject, wherein the low protein diet has less than about 15% of daily calories from whole protein.

37. The method of claim 22, further comprising administering a low protein diet to the subject, wherein the low protein diet has less than about 40 g/day of whole protein.

38. The method of claim 22, wherein the first amount of time and the second amount of time are equal.

39. The method of claim 22, wherein the first amount of time and the second amount of time are 5 days.

40. The method of claim 22, wherein the first amount of time and the second amount of time is greater than the third amount of time.

41. The method of claim 40, wherein the third amount of time is 2 days.

42. The method of claim 22, further comprising repeating steps a), b), and c).


SEQUENCE LISTING (19 sequences)

SEQ ID NO: 1 - DNA, 20 bp, Artificial Sequence
  Description of Artificial Sequence Synthetic oligonucleotide
tggacgaaggcgccctgctc

SEQ ID NO: 2 - DNA, 21 bp, Artificial Sequence
  Description of Artificial Sequence Synthetic primer
agtacagtatcctctcagaca

SEQ ID NO: 3 - DNA, 22 bp, Artificial Sequence
  Description of Artificial Sequence Synthetic primer
tcacttccaatatgatctgcca

SEQ ID NO: 4 - DNA, 24 bp, Artificial Sequence
  Description of Artificial Sequence Synthetic probe
ttctagcagccagtaaatcggggc

SEQ ID NO: 5 - DNA, 21 bp, Artificial Sequence
  Description of Artificial Sequence Synthetic primer
aggtgtctttgtcggttacag

SEQ ID NO: 6 - DNA, 19 bp, Artificial Sequence
  Description of Artificial Sequence Synthetic primer
cgtattaggggcagcagtg

SEQ ID NO: 7 - DNA, 19 bp, Artificial Sequence
  Description of Artificial Sequence Synthetic probe
ccatggcgcttctcacggc

SEQ ID NO: 8 - DNA, 19 bp, Artificial Sequence
  Description of Artificial Sequence Synthetic primer
cactgaggctgttcccatt

SEQ ID NO: 9 - DNA, 20 bp, Artificial Sequence
  Description of Artificial Sequence Synthetic primer
gtcatcaacgcagctgagaa

SEQ ID NO: 10 - DNA, 24 bp, Artificial Sequence
  Description of Artificial Sequence Synthetic probe
ccagatccacattgtccacacctg

SEQ ID NO: 11 - DNA, 20 bp, Artificial Sequence
  Description of Artificial Sequence Synthetic primer
tcatcacggacaatcaccac

SEQ ID NO: 12 - DNA, 21 bp, Artificial Sequence
  Description of Artificial Sequence Synthetic primer
gtcctcaaacttcctgtccaa

SEQ ID NO: 13 - DNA, 24 bp, Artificial Sequence
  Description of Artificial Sequence Synthetic probe
agagccaacattcttctgggcacc

SEQ ID NO: 14 - DNA, 20 bp, Artificial Sequence
  Description of Artificial Sequence Synthetic primer
catgattggagatggtgcca

SEQ ID NO: 15 - DNA, 22 bp, Artificial Sequence
  Description of Artificial Sequence Synthetic primer
ttatccttgacttgttgcctga

SEQ ID NO: 16 - DNA, 26 bp, Artificial Sequence
  Description of Artificial Sequence Synthetic probe
tgtcctcctgctgatgctttcattgg

SEQ ID NO: 17 - DNA, 17 bp, Artificial Sequence
  Description of Artificial Sequence Synthetic primer
ccttgcacatgccggag

SEQ ID NO: 18 - DNA, 17 bp, Artificial Sequence
  Description of Artificial Sequence Synthetic primer
acagagcctcgcctttg

SEQ ID NO: 19 - DNA, 21 bp, Artificial Sequence
  Description of Artificial Sequence Synthetic probe
tcatccatggtgagctggcgg